(19) United States
(12) Patent Application Publication
(10) Pub. No.: US 2023/0414748 A1
(43) Pub. Date: Dec. 28, 2023

(54) SCAFFOLDED ANTIGENS AND ENGINEERED SARS-COV-2 RECEPTOR-BINDING DOMAIN (RBD) POLYPEPTIDES

(71) Applicants: University of Florida Research Foundation, Inc., Gainesville, FL (US); Emmune, Inc., Juno Beach, FL (US)

(72) Inventors: Michael Farzan, Juno Beach, FL (US); Brian Quinlan, Jupiter, FL (US); Yan Guo, Jupiter, FL (US); Huihui Mu, Jupiter, FL (US); Wenhui He, Gainesville, FL (US); Hyeryun Choe, Juno Beach, FL (US); Lizhou Zhang, Jupiter, FL (US); Charles Bailey, Jupiter, FL (US); Michael Alpert, Jupiter, FL (US)

(21) Appl. No.: 18/036,793

(22) PCT Filed: Nov. 16, 2021
(86) PCT No.: PCT/US21/59525

§ 371 Date: May 12, 2023

Related U.S. Application Data
(60) Provisional application No. 63/114,091, filed on Nov. 16, 2020.
(60) Provisional application No. 63/232,024, filed on Aug. 11, 2021.

(51) Int. Cl. A61K 39/215; A61P 37/04

(52) U.S. Cl. CPC A61K 39/215; A61P 37/04; A61K 2039/6031

(57) ABSTRACT

The present invention provides scaffolded antigens that have demonstrated improved biochemical and immunogenic properties. The invention also provides engineered SARS-CoV-2 immunogens that contain a modified receptor-binding domain (RBD) sequence. Also provided in the invention are vaccine compositions that contain the scaffolded antigens, including the engineered RBD polypeptides that are fused to the scaffold proteins described herein. The invention also provides methods of using such vaccine compositions in various therapeutic applications, e.g., for preventing or treating SARS-CoV-2 infections.

### CROSS-REFERENCE TO RELATED APPLICATIONS

This application claims priority to US Provisional Patent Application Nos. 63/114,091 (filed Nov. 16, 2020; now pending) and 63/232,024 (filed Aug. 11, 2021; now pending). The disclosures of the priority applications are incorporated by reference in their entirety and for all purposes.

### STATEMENT OF GOVERNMENT SUPPORT

This invention was made with government support under grant number AI129868 awarded by the National Institutes of Health. The government has certain rights in the invention.

### BACKGROUND OF THE INVENTION

Coronaviruses (CoV) are enveloped viruses with a positive-stranded RNA genome. Several coronaviruses are pathogenic in humans. Among these, SARS coronavirus 2 (SARS-CoV-2) is a highly transmissible and virulent coronavirus that is the cause of an ongoing global pandemic. SARS-CoV-2 and other related coronaviruses infect host cells by binding to their common receptor, angiotensin converting enzyme 2 (ACE2), with their respective spike (S) protein. A discrete ˜197-amino-acid domain of the S protein, named either SB or the receptor-binding domain (RBD), directly associates with ACE2.

While several vaccines have been officially approved around the world for preventing human SARS-CoV-2 infection in the past few months, there is still an ongoing and urgent need for additional vaccines that are effective for countering the coronavirus, including SARS-CoV-2 variants that continue to emerge. The present invention is directed to this and other unmet needs.

### SUMMARY OF THE INVENTION

In one aspect, the invention provides engineered antigens or immunogen polypeptides that are derived from SARS-CoV-2 spike (S) protein. These antigens contain an altered receptor-binding domain (RBD) sequence of the S protein that has modifications relative to the wildtype RBD sequence. The modifications include mutations at the inter-subunit interfaces of the RBD that result in (a) formation of at least two engineered N-linked glycosylation sites, (b) formation of at least one engineered N-linked glycosylation site and substitution of at least one additional hydrophobic residue at the inter-subunit interface, or (c) formation of at least one engineered N-linked glycosylation site that is formed from two substitutions. In some embodiments, the wildtype RBD sequence that was mutated contain residues N331-P527 of SARS-CoV-2 S protein sequence of Access No. YP_009724390.1 (SEQ ID NO:2) or a substantially identical or conservatively modified variant thereof. In various embodiments, the mutations introduced into the wildtype sequence that result in the formation of an N-linked engineered glycosylation site include V362(S/T), L517N/H519(S/T), A520N/P521X/A522(S/T), A372T, A372S, Y396T, D428N, R357N/S359T, R357N/S359S, S371N/S373T, S371N/S373S, S383N/P384V, S383N/P384A, S383N/P384I, S383N/P384L, S383N/P384M, S383N/P384W, K386N/N388T, K386N/N388S, and G413N. In these substitutions, X is any amino acid except for P.

In some embodiments, the engineered antigen has substitution of at least one additional hydrophobic residue in V367, A372, L390, L455, L517, L518, A520 or A522 with a charged amino acid residue. In some of these embodiments, the substituting charged amino acid residue is Asp or Glu. In some embodiments, mutations in the engineered antigen include (a) any two of A372(T/S), and L517N/H519(T/S), (b) L517N/H519(T/S) and D428N, (c) any three of A372(T/S), Y396T, D428N, and L517N/H519(T/S), (d) any two of A372(T/S), Y396T, D428N, and L517N/H519(T/S), plus substitution of L518; (e) any two of A372(T/S), Y396T, and D428N, plus substitution of L517; (f) L517N/H519(T/S), plus substitution of V372, (g) L517N/H519(T/S), plus substitution of L390; or (h) any two of V362(S/T), A372(S/T), D428N, L517N/H519(T/S), A520N/P521X/A522(S/T), wherein X is any amino acid except for P. In some embodiments, the mutations in the engineered RBD antigen include substitutions L517N/H519T or L517N/H519S in the wildtype RBD sequence (SEQ ID NO:2). In some of these embodiments, the engineered antigen further contains one or more substitutions selected from the group consisting of D428N, A372(T/S), Y396T, V372(D/E), L390(D/E), L455A and L518(D/E/G/S). In some embodiments, the engineered antigen can further contain two or more substitutions selected from the group consisting of V362(S/T), D428N, L518(D/E/G/S). As exemplifications, some engineered RBD immunogen polypeptides of the invention contain the amino sequence shown in any one of SEQ ID NOs:3, 162-168 and 241-246, or a substantially identical or conservatively modified variant thereof. In various embodiments, the engineered RBD antigens of the invention do not contain a full-length SARS-CoV-2 spike (S) protein.

In another aspect, the invention provides fusion proteins that contain an antigen and a scaffold protein. In the fusion protein, the scaffold protein is at least 50% (e.g., at least 60%, at least 65%, at least 70%, at least 75%, at least 80%, at least 85%, at least 90%, at least 95%, at least 96%, at least 97%, or at least 98%) identical to amino acids 2-96 of Acidiferrobacteraceae bacterium (Ap) half-ferritin (SEQ ID NO: 10). In some of these embodiments, the C-terminus of the scaffold protein is fused (a) to the N-terminus of the antigen directly, (b) to the N-terminus of the antigen through a polypeptide linker, or (c) to the antigen via an isopeptide bond. Some of the fusion proteins contain the sequence shown in SEQ ID NO:10, or a substantially identical or conservatively modified variant thereof. In some other embodiments, the employed scaffold protein in the fusion proteins contains a sequence that is at least 50% (e.g., at least 60%, at least 65%, at least 70%, at least 75%, at least 80%, at least 85%, at least 90%, at least 95%, at least 96%, at least 97%, or at least 98%) identical to the F10 protein sequence shown in any one of SEQ ID NOs:169-240. Some of these fusion proteins contain an amino acid sequence shown in any one of SEQ ID NOs:169-240, or a substantially identical or conservatively modified variant thereof. In some fusion proteins of the invention, the employed scaffold protein is a self-assembling homo-multimer comprising 10-59 subunits. In some embodiments, the C-terminus of the scaffold protein is fused (i) to the N-terminus of the antigen directly, or (ii) to the N-terminus of the antigen through a polypeptide linker.

In a related aspect, the invention provides fusion proteins that contain an engineered RBD immunogen polypeptide described herein and at least part of a heterologous protein. Some of these fusion proteins contain a transmembrane region or a glycosylphosphatidylinositol (GPI) anchor signal sequence. In some of the fusion proteins, the heterologous protein is a self-assembling multimer scaffold protein.

In another aspect, the invention provides fusion proteins that contain a scaffold protein sequence and an antigen of interest. In these embodiments, the scaffold protein is a self-assembling homo-multimer comprising 13-59 subunits, and the C-terminus of the scaffold protein is fused (i) to the N-terminus of the antigen directly, (ii) to the N-terminus of the antigen through a peptide or polypeptide linker, or (iii) to the antigen via an isopeptide bond. In some of these embodiments, self-assembly of the scaffold protein is not dependent upon cysteine coordination of a metal ion or binding to nucleic acid. In some of the fusion proteins, the antigen of interest contains an altered receptor-binding domain (RBD) sequence of SARS-CoV-2 spike (S) protein that has modifications relative to the wildtype RBD sequence. The modifications in the altered RBD sequence contain mutations at the inter-subunit interfaces of the RBD that result in (a) formation of at least two engineered N-linked glycosylation sites or (b) formation of at least one engineered N-linked glycosylation site and substitution of at least one additional hydrophobic residue at the inter-subunit interface.

In various embodiments, the fusion proteins of the invention can include an N-terminal signal sequence for secretion into the endoplasmic reticulum (ER) of a mammalian cell. In some of the fusion proteins, the scaffold protein is not an ATPase or a heat-shock protein. In some of the fusion proteins, the employed scaffold protein is a self-assembling homo-multimer comprising 24-48 subunits. In some embodiments, the scaffold protein is a substantially identical or conservatively modified variant of a protein from a prokaryote. In some embodiments, the scaffold protein is a substantially identical or conservatively modified variant of a protein from a thermophile or hyperthermophile.

In various embodiments, the scaffold protein of the fusion proteins of the invention can contain at least one N-linked glycan. In some of the fusion proteins of the invention, the employed scaffold protein is an imidazoleglycerol-phosphate dehydratase (HisB) protein or a substantially identical or conservatively modified variant thereof. In some of these embodiments, the scaffold protein contains at least one N-linked glycan. In various embodiments, the scaffold protein contains at least one N-linked glycan (a) in the region corresponding to positions 1-59 of SEQ ID NO:34 or (b) at the position corresponding to 12 of SEQ ID NO:34. In some other fusion proteins of the invention, the employed scaffold protein is an ATP-dependent Clp protease proteolytic subunit (ClpP) protein, a catalytically-inactive ClpP protein, or a substantially identical or conservatively modified variant thereof. In some of these embodiments, the scaffold protein contains at least one N-linked glycan. In some embodiments, the scaffold protein contains a valine residue at the position corresponding to A140 of SEQ ID NO:97. In various fusion proteins of the invention, the employed scaffold protein contains the sequence shown in any one of SEQ ID NO:4-10 and 34-154, or a substantially identical or conservatively modified variant thereof. Some specific fusion proteins of the invention contain the sequence shown in any one of SEQ ID NOs:11-22, or a substantially identical or conservatively modified variant thereof. In another aspect, the invention provides vaccine compositions that contain two or more distinct versions of a fusion protein described herein.

In some related aspects, the invention provides polynucleotides that encode the various engineered antigens or fusion proteins described herein. In some embodiments, the polynucleotides of the invention are ribonucleic acid (RNA) molecules. In some aspects, the invention also provides SARS-CoV-2 vaccine compositions that contain one or more of the engineered antigens disclosed herein, or one or more of the disclosed fusion proteins harboring an engineered RBD polypeptide described herein, or that contains a polynucleotide described herein. In some embodiments, the SARS-CoV-2 vaccine composition contains two or more distinct versions of the engineered antigen, two or more distinct versions of the fusion protein, or two or more distinct versions of the polynucleotide. The invention also provides pharmaceutical compositions that contain such a vaccine composition and a pharmaceutically acceptable carrier. The invention additionally provides diagnostic kits for using the engineered RBD polypeptides or related fusion proteins in the detection of antibodies that bind to SARS-CoV-2 (e.g., to RBD). Related methods for detecting such antibodies are also provided. Further provided in the invention are therapeutic methods for preventing or treating a coronavirus infection in a subject. These methods entail administering to the subject a pharmaceutically effective amount of a vaccine composition or a pharmaceutical composition described herein.

A further understanding of the nature and advantages of the present invention may be realized by reference to the remaining portions of the specification and claims.

### DESCRIPTION OF THE DRAWINGS

FIG. 1 shows engineered glycosylations of the SARS-CoV-2 RBD to enable expression as multimeric antigen fusion proteins. Views of the RBD (A) in the context of the Spike in the open one-up conformation and (B) bound to the ACE2 receptor. Black indicates the ACE2-binding surface. Light gray (regions proximal to L517 and Y396) indicates surfaces of the RBD that are occluded in the native Spike trimer. Dark gray indicates surface residues that are neither occluded in closed conformation nor part of the ACE2 interface). White residues are positions of mutations where glycosylations have been engineered. (C) The sequence of the hyper-glycosylated RBD (gRBD) (SEQ ID NO:3). Glycosylation motifs (2 native and 4 engineered) are underlined (dark gray shading indicates the ACE2-binding region and light gray shading indicates the sites of mutations introduced in gRBD).

FIG. 2 shows that SARS-CoV-2 RBD nanoparticles are strongly immunogenic. Four female Sprague Dawley rats for each group were inoculated with either RBD-Spytag or S-protein-Spytag conjugated to either Spycatcher-I3 particles (A) by isopeptide bond formation, or KLH (B) by EDC. The indicated dilutions of preimmune sera (day 0) were compared to dilutions of sera harvested from immunized rats at day 40. Each serum was compared for its ability to neutralize S-protein-pseudotyped retroviruses (SARS2-PV), by measuring the activity of a firefly-luciferase reporter expressed by these pseudoviruses. The figure shows entry of SARS2-PV as a percentage of that observed without added rat serum. Error bars indicate s.d. for biological replicates. (C) IC80 values for each rat at day 40 were calculated in Prism 8 and significance between groups is indicated (* indicates P<0.05; ** indicates P<0.01; ns indicates P>0.05; one-way ANOVA with Tukey's multiple comparison test)

FIG. 3 shows expression of gRBD as a membrane associated Fc-fusion protein four-fold greater than the analogous wild-type RBD construct. “gRBD”, a variant modified so that it includes four glycosylation sites away from the ACE2 and antibody-binding region of the RBD. The wild-type RBD and gRBD were each fused to an Fc domain connected to an exogenous transmembrane domain (of PDGFR) and transfected into HEK239T cells. Cells were then stained with anti-Fc (to recognize total expression) or ACE2-Fc to validate appropriate folding of the RBD. Note the four-fold greater expression of folded RBD with the gRBD variant.

FIG. 4 shows substantially greater expression of gRBD than wild-type RBD when fused to multimerizing scaffolds. Fusion constructs of wild-type RBD or gRBD were made with the mi3 60-mer were expressed from transfected HEK293T and detected by Western blot with an anti-tag antibody (A) or by ELISA with ACE2-Ig (B). Note that total expression of the wild-type RBD-mi3 construct is lower as indicated in cell lysates, and less is secreted as indicated by cell supernatants. The amino-acid sequence of the construct used in these studies is shown in SEQ ID NO:3. The wild-type RBD and various gRBD constructs derived from the SARS-CoV-2 reference strain (C) or beta variant (D) RBDs were fused to the C-terminus of the F10 scaffold and expressed in HEK293 Ts, expressed in HEK293T transfections, and detected in supernatants by ELISA. gRBD.1 derived from the reference strain also was expressed as fusions to F10, NAP, SE, SaClpP, CtHisB, and SaHisB, expressed in HEK293T transfections, and detected in supernatants by ELISA (E).

FIG. 5 shows optimization of an engineered RBD for multimeric expression. SARS-CoV2 RBD variants with different combinations of glycosylations were expressed as fusions to the C-terminus of HP-NAP. Native western blots probed with ACE2-Fc-HRP were performed on Expi293 supernatants 5 (A) or 3 (B) days post transfection. The minimum necessary glycosylation for efficient particle expression is the glycosylation at 517 (B lane 1). Other glycosylations serve to enhance expression or suppress higher order aggregates.

FIG. 6 shows expression of several scaffolded or multimerized RBD constructs, including gRBD-Fc, gRBD-foldon, NAP-gRBD, gRBD-ferritin and gRBD-mi3. (A) Blue-Native PAGE of purified wtRBD and gRBD expressed on diverse multimerization platforms, 5 μg/well. wtRBD did not express on the mi3 platform. (B) Yields of purified wtRBD and gRBD multimers expressed from the CMVR vector in Expi293 cells. Values stated are from a minimum of two independent transfections. Error bars represent S. D. The actually expressed gRBD-foldon and NAP-gRBD contain SEQ ID NO:12 and 13, respectively, plus a C-tag at the C-terminus. The actually expressed gRBD-ferritin protein contains SEQ ID NO:14 and an N-terminal FLAG tag. The actual expressed gRBD-mi3 protein contains SEQ ID NO:15 and a SnoopTag/C-Tag at the C-terminus.

FIG. 7 shows that gRBD based DNA vaccines more efficiently raise neutralizing antibodies than those based on wild-type RBD. Five mice per group were electroporated with 60 μg/hind leg of plasmid DNA expressing wtRBD or gRBD fused to human Fc dimer (A), foldon trimer (B), Helicobacter pylori NAP 12-mer (C), Helicobacter pylori ferritin 24-mer (D), and mi3 60-mer (E). An additional control group was electroporated with plasmid expressing SARS-CoV2 spike protein with two stabilizing prolines (F). Electroporations were conducted day 0 and day 14, and serum was collected and pooled for neutralization assays on day 21. Pooled preimmune sera, and pooled preimmune sera doped with 200 μg/mL of ACE2-Fc were used as negative and positive controls. (G) Neutralizing potency varied by platform. (H) IC50 calculations for wtRBD and gRBD were calculated (Prism 8) against normalized values by least squares fit. P-value was calculated by 2-tailed paired t test between wtRBD and gRBD pairs.

FIG. 8 shows that gRBD is inherently more immunogenic than wild-type. Five mice per group were inoculated with 25 μg of protein A/SEC purified wtRBD-Fc or gRBD-Fc adjuvanted with 25 μg of MPLA and 10 μg QS-21. Immunizations were conducted day 0 and day 14, and serum was collected and pooled on day 21. Pooled preimmune sera, and pooled preimmune sera doped with 200 μg/mL of ACE2-Fc were used as negative and positive controls. (A) SARS-CoV-2 pseudovirus neutralizations. (B) LCMV pseudovirus control neutralizations. HEK-293T cells were transfected with 1 μg/well in a six well plate and stained the next day with pooled preimmune, and day 21 sera and then stained with either (C) anti-mouse-FITC or (D) ACE2-Fc-DyLight650.

FIG. 9 shows that fusion of gRBD to the C-terminus of fusion platforms results in better assembled particles than fusion to the N-terminus. wtRBD and gRBD form better assembled particles fused to the C-termini diverse platforms as assessed by Blue Native PAGE 5 μg/well (A) The 12-mer NAP protein from Helicobacter pylori has very low aggregation with gRBD fused to the C-terminus but not the N-terminus. (B) The 12-mer dodecin from Bordetella pertussis (BpDoD) assembles well with gRBD fused to the C-terminus but not the N-terminus.

FIG. 10 shows self-assembling multimer platforms that allow C-terminal fusion. Diverse multimeric platforms with available C-termini display gRBD in well behaved particles as assessed by Blue Native PAGE 5 μg/well. Bacterial encapsulated ferritin from Acidiferrobacteraceae bacterium (AbEF) and a Dps from Salmonella Enterica (SeDps) display gRBD at the C-terminus with low aggregation (A), as do Archaeal encapsulated ferritins from Pyrococcus yayanosii and Thermoplasmata archaeon (B). Larger multimer platforms with a free C-terminus. The 24-mer HisB and the 14-mer ClpP, both from Staphylococcus aureus (C) can also be used to display gRBD at high yield and low aggregation.

FIG. 11 shows HisB expression as a multimer, and assembly and disassembly of HisB trimers into multimers. Staphylococcus aureus HisB (SaHisB) was used as the scaffold. SaHisB-gRBD nanoparticles self-assembled with high-fidelity into 24-mer multimers, and were effectively separated from unassembled trimers by Size Exclusion Chromatography (Superose 6 Increase) (A). The homogeneity of 24-mer assembly was visualized by Native Blue PAGE. Blue Native PAGE of 5 μg of SaHisB-gRBD incubated with 1 mM MnCl₂, no additive or 10 mM EDTA in 15 μl for 72 hours at 4° C. prior to addition of loading buffer and electrophoresis shows assembly in the presence of MnCl₂ and disassembly in the presence of EDTA of HisB trimers into multimers (B).

FIG. 12 shows ClpP and HisB scaffold multimer assembly fidelity and immunofocusing improvements. Variants of ClpP (A) and HisB (B) were expressed with gRBD fused to the C-termini. Native western blots probed with ACE2-Fc-HRP were performed on Expi293 supernatants 3 days post transfection. The A140V space-filing mutation stabilizes the 14-mer form of ClpP without loss of yield (A). Addition of an outward facing glycosylation using the double mutant 12N+Q4T on SaHisB does not lead to a loss of yield (B).

FIG. 13 shows a phylogenetic tree of the HisB orthologs from various organisms. The tree includes HisB protein sequences from bacteria, archaea, and fungi that are mesophiles, thermophiles, and hyperthermophiles.

FIG. 14 shows a phylogenetic tree of the ClpP orthologs from various organisms. The tree includes ClpP protein sequences from bacteria, archaea, and fungi that are mesophiles, thermophiles, and hyperthermophiles.

FIG. 15 shows the protein yields and multimerization fidelity for a series of F10-gRBD fusion proteins. The F10-gRBD fusion proteins contain the engineered glycans as indicated in Table 3. Such F10-gRBD fusion proteins were generated that were based on the Reference/Wuhan RBD sequence (SEQ ID NO:2), or based on the Beta/South Africa RBD sequence (SEQ ID NO: 158). The protein yields generated by transient transfection of Expi293 cells with these protein variants are shown (A). Multimerization fidelity was assessed by native protein gel electrophoresis (native PAGE) for the F10-gRBD proteins based on the Reference/Wuhan RBD sequence (B) or the Beta/South Africa RBD sequence (C).

FIG. 16 shows the results of DNA vaccination and recombinant protein vaccination experiments that include the F10 scaffold. DNA vaccinations (A). Five mice per group were electroporated in each hind leg with 60 μg plasmid DNA of gRBD.1 fused to human Fc dimer (circles), H. pylori ferritin (24-mer; down triangles), S. aureus HisB (24-mer; squares), F10 (radial 10-mer, diamonds), and S. aureus ClpP (radial 14-mer, up triangles). Pooled preimmune sera (stars) was used as a negative control. Protein vaccinations (B). Five mice per group were inoculated twice at a 2 week interval with 1 μg of protein antigen, 5 μg QuilA and MPLA adjuvants with the indicated column purified gRBD.1-scaffold variants. Pooled preimmune sera was used as a negative control. IC50s for both figures were calculated with Prism 8 against normalized values by least-squares fit. Error bars represent 95% confidence values. The F10 scaffold consistently matched or surpassed the immunogenicity ClpP and HisB as well as roughly six other novel scaffolds (not shown) in both DNA- and adjuvanted protein-based vaccines.

FIG. 17 shows the results of an experiment assessing the ability of F10-gRBD to tolerate lyophilization. F10-gRBD.1 or F10-gRBD.5 fusions were lyophilized in 0.5M Trehalose. Lyophilized proteins were either heat stressed at 45° C. for 2 days or maintained frozen at minus 80° C. After resuspension, protein was analyzed on a BlueNative gel (A) or by a native western using ACE2-HRP (B). Note that in all cases the F10 decamer remained fully assembled (band at 720 kDa), and that heat stress and frozen material bound ACE2 with equal efficiencies. The antigens shown in panels A and B were inoculated twice at a 3-week interval into five mice per group with 2.5 μg of reconstituted lyophilized protein, 5 μg of QuilA and 5 μg MPLA, and analyzed by pseudovirus neutralization with a D614G-modified Index (Wuhan) S protein (C). IC50 serum dilutions were assayed with Index-D614G pseudoviruses derived from the Reference strain or B.1.351 (D). Excepting the −80° C. comparisons between D614 and Beta, none of the differences observed in C and D were statistically significant.

FIG. 18 shows the production, purification, and immunogenicity of F10-gRBD in the baculovirus/Sf9-cell system. F10-gRBD.5-expressing baculovirus (flashBAC Ultra) were used to infect ExpiSF cells. Supernatants were collected 2 days later, clarified by centrifugation, and run through Sartobind S (to pre-clear baculovirus media) and Sartobind Q ion-exchange columns (first enrichment, to 85% purity) (A). Both columns were eluted with Tris 7.5 1M NaCl, and buffer was exchanged to TBS 0.15 M NaCl. Eluates and flow through were examined by Blue Native PAGE. Note the lack of F10-gRBD.5 in flow through, indicating no loss of material. Sartobind Q eluates were further purified by SEC (not shown) for studies in panels B and C. Neutralization studies using Index-D614 or Beta (B). Purified F10-gRBD.5 produced in Exp293 or ExpiSF systems were lyophilized in 0.5M Trehalose as in FIG. 16. Five mice per group were inoculated twice at a 3 week interval with 2.5 μg of reconstituted lyophilized protein, 5 μg of QuilA and MPLA, and analyzed as in FIG. 16C. IC50s were calculated as in FIG. 16D. Differences between Expi239 and Sf9-produced antigens were significant (p<0.05) (C).

FIG. 19 shows the phylogenetic relationships of F10 proteins from various thermophilic bacteria and archaea.

FIG. 20 shows the phylogenetic relationships of various prokaryotic F10 proteins.

FIG. 21 shows an amino acid sequence alignment for various prokaryotic F10 proteins. The sequences shown are SEQ ID NOs:10 and 169-240, respectively.

### DETAILED DESCRIPTION

I Overview

The viral genome of SARS-CoV-2 encodes spike (S), envelope (E), membrane (M), and nucleocapsid (N) structural proteins, among which the S glycoprotein is responsible for binding the host receptor via the receptor-binding domain (RBD) in its S1 subunit, as well as the subsequent membrane fusion and viral entry driven by its S2 subunit. A possible membrane fusion process has been proposed. The receptor binding may help to keep the RBD in a ‘standing’ state, which facilitates the dissociation of the S1 subunit from the S2 subunit.

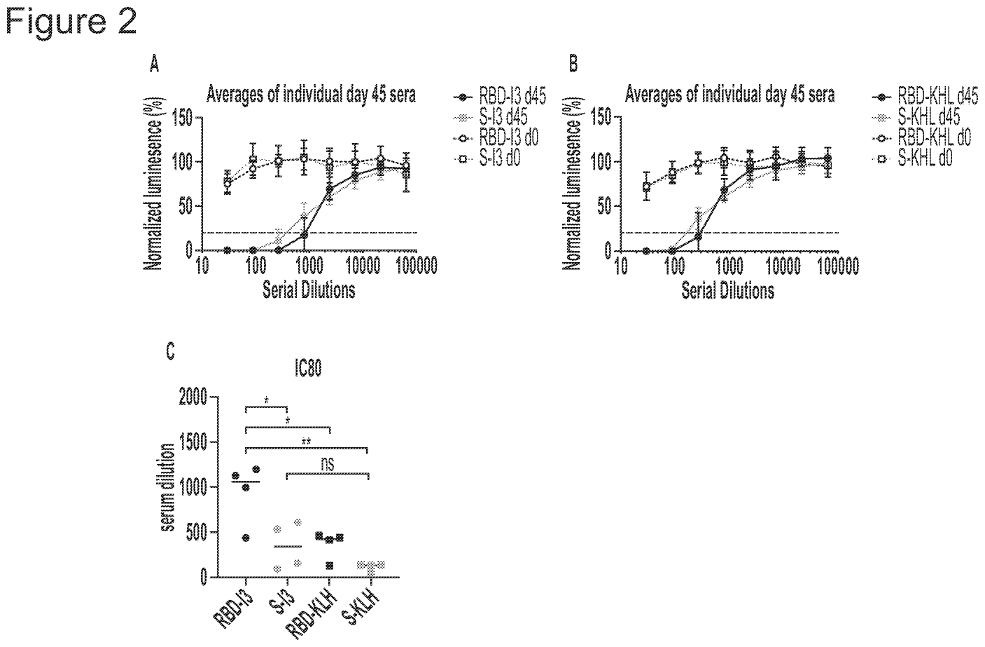

The RBD is the major, if not the sole, neutralizing epitope on the SARS-CoV-2 spike (S) protein, and it elicits more neutralizing antibodies than the whole S protein (FIG. 2). While RBD has been the focus of SARS-CoV-2 vaccine development, monomeric RBD is unlikely to make a potent vaccine because of its small size, its inability to crosslink the B-cell receptor or activate complement, or to stay bound in follicular dendritic cells in the lymph node. Thus, to be expressed as part of a vaccine, it should be expressed as a multimer. However, the wild-type RBD expresses on multimerizing carriers like bacterioferritin, hepatitis B core, or mi3 very poorly, probably because it tends to aggregate.

The present invention is predicated in part on the studies undertook by the inventors to identify structural motifs of SARS-CoV-2 that could provide effective vaccine immunogens epitope for generating neutralizing antibodies. As detailed herein, it was identified by the inventors that the RBD is sufficient as a SARS-CoV vaccine and does not raise enhancing antibodies that could decrease the safety or efficacy of such a vaccine. Also, the inventors engineered RBD polypeptides that aggregate less and expresses more efficiently than the native RBD. It was found that the engineered RBD has properties especially useful when it is expressed as a multimer, for example as a fusion scaffold with ferritin or mi3 multimerizing scaffold. Specifically, it was observed that little or no wild-type RBD is produced as a mI3 or ferritin fusion, whereas fusions of multimerizing scaffolds with the engineered RBD express efficiently. These multimerizing scaffolds enhance immunogenicity over monomeric RBD, with robust responses shown with a conjugated multimer. Results from these studies indicate that the engineered RBD polypeptides would enable the expression and simplifies production of immunogenic fusion constructs not possible with the native RBD, a significant advantage for vaccines produced as recombinant proteins, and those delivered as mRNA or with a viral vector. In addition, the inventors found that the engineered RBD expressed more efficiently than the wild-type RBD when expressed on the cell surface, e.g., with a transmembrane protein anchor.

The invention is further predicated in part on the studies undertook by the inventors to identify multimerizing scaffolds for the expression of the RBD as a multimeric antigen. These studies led to the observation that self-assembling homo-multimer scaffolds with available C-termini displayed on the exterior of the scaffold multimer generally possessed greater potential for expression and homogeneity when fused to the RBD antigen than similar constructs where the N-terminus of the scaffold is fused to the RBD antigen. Additionally, it was found that multimers with a number of subunits within the range of 12-60 subunits, e.g., 24-48 subunits, expressed and elicited immune responses most efficiently. As exemplifications, several novel scaffolds were identified, including ClpP and HisB, each of which have numerous orthologs.

The invention provides novel coronavirus immunogens, scaffolded antigens, and vaccine compositions in accordance with the studies and exemplified designs described herein. In particular, the present invention includes engineered RBD molecules, protein scaffolds, and fusion proteins containing a protein scaffold described herein and an antigen. Some of the fusion proteins are vaccine antigens for SARS-CoV-2 based on fusion proteins containing a scaffold and an engineered RBD described herein. Related polynucleotide sequences, expression vectors and pharmaceutical compositions are also provided in the invention. In various embodiments, the engineered RBD proteins, in the forms of protein or nucleic acid (e.g., DNA or mRNA) carried by a viral vector can be used as coronavirus vaccines. In addition, nanoparticles presenting the engineered RBDs in multimeric format can be used as VLP-type coronavirus vaccines. Also provided in the invention are therapeutic methods of using the vaccine compositions described herein for preventing and/or treating SARS-CoV-2 infections.

Unless otherwise specified herein, the vaccine immunogens of the invention, the encoding polynucleotides, expression vectors and host cells, as well as the related therapeutic applications, can all be generated or performed in accordance with the procedures exemplified herein or routinely practiced methods well known in the art. See, e.g., Methods in Enzymology, Volume 289: Solid-Phase Peptide Synthesis, J. N. Abelson, M. I. Simon, G. B. Fields (Editors), Academic Press; 1st edition (1997) (ISBN-13: 978-0121821906); U.S. Pat. Nos. 4,965,343, and 5,849,954; Sambrook et al., Molecular Cloning: A Laboratory Manual, Cold Spring Harbor Press, N.Y., (3^{rd} ed., 2000); Brent et al., Current Protocols in Molecular Biology, John Wiley & Sons, Inc. (ringbou ed., 2003); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998). The following sections provide additional guidance for practicing the compositions and methods of the present invention.

Unless otherwise noted, the expression “at least” or “at least one of” as used herein includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

Unless otherwise noted, the order of steps or order for performing certain actions is immaterial so long as the present invention remain operable. Moreover, two or more steps or actions may be conducted simultaneously.

Unless otherwise noted, the use of any and all examples, or exemplary language herein, for example, “such as” or “including,” is intended merely to illustrate better the present invention and does not pose a limitation on the scope of the invention. No language in the specification should be construed as indicating any non-claimed element as essential to the practice of the present invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein. Unless otherwise noted, the term “vaccine immunogen” is used interchangeably with “protein antigen” or “immunogen polypeptide”.

The term “conservatively modified variant” applies to both amino acid and nucleic acid sequences. With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Epitope refers to an antigenic determinant. These are particular chemical groups or peptide sequences on a molecule that are antigenic, such that they elicit a specific immune response, for example, an epitope is the region of an antigen to which B and/or T cells respond. Epitopes can be formed both from contiguous amino acids or noncontiguous amino acids juxtaposed by tertiary folding of a protein.

Effective amount of a vaccine or other agent that is sufficient to generate a desired response, such as reduce or eliminate a sign or symptom of a condition or disease, such as pneumonia. For instance, this can be the amount necessary to inhibit viral replication or to measurably alter outward symptoms of the viral infection. In general, this amount will be sufficient to measurably inhibit virus (for example, SARS-CoV-2) replication or infectivity. When administered to a subject, a dosage will generally be used that will achieve target tissue concentrations that has been shown to achieve in vitro inhibition of viral replication. In some embodiments, an “effective amount” is one that treats (including prophylaxis) one or more symptoms and/or underlying causes of any of a disorder or disease, for example to treat a coronavirus infection. In some embodiments, an effective amount is a therapeutically effective amount. In some embodiments, an effective amount is an amount that prevents one or more signs or symptoms of a particular disease or condition from developing, such as one or more signs or symptoms associated with coronaviral infections.

Unless otherwise noted, a fusion protein is a recombinant protein containing amino acid sequence from at least two unrelated proteins that have been joined together, via a peptide bond, to make a single protein. The unrelated amino acid sequences can be joined directly to each other or they can be joined using a linker sequence. As used herein, proteins are unrelated, if their amino acid sequences are not normally found joined together via a peptide bond in their natural environment(s) (e.g., inside a cell). For example, the amino acid sequences of bacterial Thermotoga maritima encapsulin (from which mi3 60-mer is derived) and the amino acid sequences of the RBD domain of a coronavirus S glycoprotein are not normally found joined together via a peptide bond.

Glycosylation, the attachment of sugar moieties to proteins, is a post-translational modification (PTM) that provides greater proteomic diversity than other PTMs. Glycosylation is critical for a wide range of biological processes, including cell attachment to the extracellular matrix and protein-ligand interactions in the cell. This PTM is characterized by various glycosidic linkages, including N-, O- and C-linked glycosylation, glypiation (GPI anchor attachment), and phosphoglycosylation. Glycoproteins can be detected, purified and analyzed by different strategies, including glycan staining and visualization, glycan crosslinking to agarose or magnetic resin for labeling or purification, or proteomic analysis by mass spectrometry, respectively.

Sequence identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Homologs or orthologs of nucleic acid or amino acid sequences possess a relatively high degree of sequence identity/similarity when aligned using standard methods. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

SpyCatcher-SpyTag refers to a protein ligation system that is based on based on the internal isopeptide bond of the CnaB2 domain of FbaB, a fibronectin-binding MSCRAMM and virulence factor of Streptococcus pyogenes. See, e.g., Terao et al., J. Biol. Chem. 2002; 277:47428-47435; and Zakeri et al., Proc. Natl. Acad. Sci. USA. 2012; 109:E690-E697. It utilizes a modified domain from a Streptococcus pyogenes surface protein (SpyCatcher), which recognizes a cognate 13-amino-acid peptide (SpyTag). Upon recognition, the two form a covalent isopeptide bond between the side chains of a lysine in SpyCatcher and an aspartate in SpyTag. This technology has been used, among other applications, to create covalently stabilized multi-protein complexes, for modular vaccine production, and to label proteins (e.g., for microscopy). The SpyTag system is versatile as the tag is a short, unfolded peptide that can be genetically fused to exposed positions in target proteins; similarly, SpyCatcher can be fused to reporter proteins such as GFP, and to epitope or purification tags.

A similar system, SnoopCatcher-SnoopTag, has been developed based on another Gram-positive surface protein, the pilus adhesin RrgA of S. pneumoniae. The D4 domain of this protein is stabilized by an isopeptide forming between a lysine (K742) and an asparagine (N854), catalyzed by the spatially adjacent E803. This domain was split into a scaffold protein called SnoopCatcher and a 12-residue peptide termed SnoopTag, which can spontaneously form a covalent isopeptide bond upon mixing. In contrast to SpyCatcher-SpyTag, the reactive lysine is present in SnoopTag and the asparagine in SnoopCatcher. This system is orthogonal to SpyCatcher-SpyTag; that is, SnoopCatcher does not react with SpyTag and SpyCatcher does not react with SnoopTag. This allows the use of both systems simultaneously to produce “polyproteams,” programmed modular polyproteins.

The term “subject” refers to any animal classified as a mammal, e.g., human and non-human mammals. Examples of non-human animals include dogs, cats, cattle, horses, sheep, pigs, goats, rabbits, and etc. Unless otherwise noted, the terms “patient” or “subject” are used herein interchangeably. Preferably, the subject is human.

The term “treating” or “alleviating” includes the administration of compounds or agents to a subject to prevent or delay the onset of the symptoms, complications, or biochemical indicia of a disease (e.g., A CORONAVIRUS infection), alleviating the symptoms or arresting or inhibiting further development of the disease, condition, or disorder. Subjects in need of treatment include those already suffering from the disease or disorder as well as those being at risk of developing the disorder. Treatment may be prophylactic (to prevent or delay the onset of the disease, or to prevent the manifestation of clinical or subclinical symptoms thereof) or therapeutic suppression or alleviation of symptoms after the manifestation of the disease.

Vaccine refers to a pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In some embodiments of the invention, vaccines or vaccine immunogens or vaccine compositions are expressed from fusion constructs and self-assemble into nanoparticles displaying an immunogen polypeptide or protein on the surface.

Virus-like particle (VLP) refers to a non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art. The presence of VLPs following recombinant expression of viral proteins can be detected using conventional techniques known in the art, such as by electron microscopy, biophysical characterization, and the like. See, for example, Baker et al. (1991) Biophys. J. 60:1445-1456; and Hagensee et al. (1994) J. Virol. 68:4503-4505. For example, VLPs can be isolated by density gradient centrifugation and/or identified by characteristic density banding. Alternatively, cryoelectron microscopy can be performed on vitrified aqueous samples of the VLP preparation in question, and images recorded under appropriate exposure conditions.

A self-assembling nanoparticle refers to a ball-shape protein shell with a diameter of tens of nanometers and well-defined surface geometry that is formed by identical copies of a non-viral protein capable of automatically assembling into a nanoparticle with a similar appearance to VLPs. Known examples include ferritin (FR), which is conserved across species and forms a 24-mer, as well as B. stearothermophilus dihydrolipoyl acyltransferase (E2p), Aquifex aeolicus lumazine synthase (LS), and Thermotoga maritima encapsulin, which all form 60-mers. Self-assembling nanoparticles can form spontaneously upon recombinant expression of the protein in an appropriate expression system. Methods for nanoparticle production, detection, and characterization can be conducted using the same techniques developed for VLPs.

Full-length SARS-CoV-2 Spike (S) protein means a protein containing at least amino acids 16-1213 of the sequence of SEQ ID NO:1 or a substantially identical or conservatively modified variant thereof.

### III. Engineered SARS-CoV-2 RBD Immunogen Polypeptides

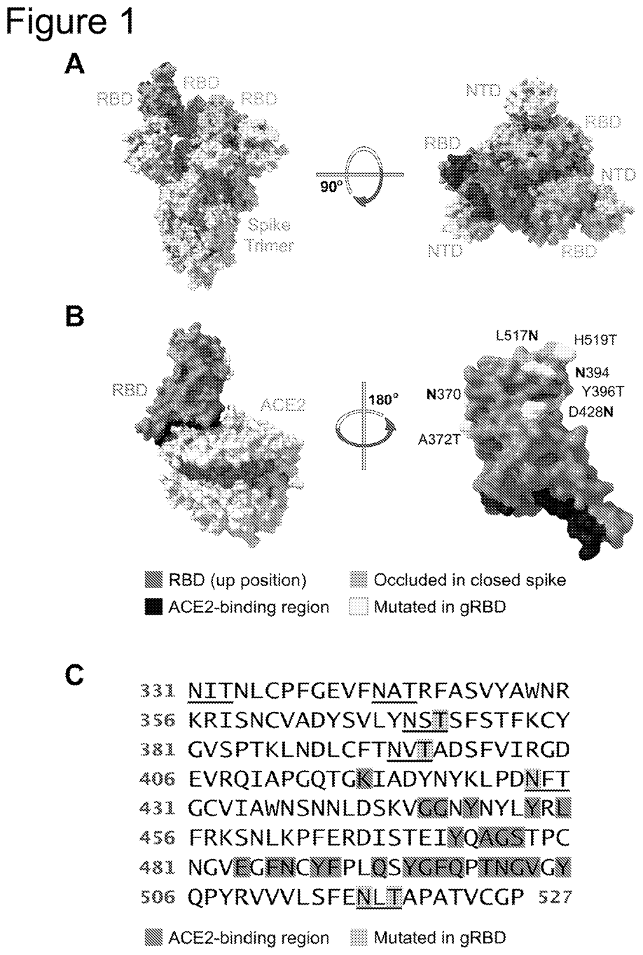

The invention provides engineered SARS-CoV-2 RBD polypeptide sequences that are suitable for developing vaccines. As detailed herein, biological and immunogenic properties (e.g., stability, purity, expression yield, and antibody response) of the engineered RBD immunogens are substantially improved over the wildtype RBD sequence. The SARS-CoV-2 spike (S) protein is a trimer containing domains that include the RBD and the N-terminal domain (NTD). When the RBD is in the ‘down’ position, it makes direct contacts with other subunits, including the NTD and other RBDs, across inter-subunit interfaces (FIG. 1A). In general, the engineered RBD polypeptides contain one or more amino acid substitutions, relative to the wildtype RBD sequence, that result in formation of one or more novel glycosylation sites that occlude residues at the inter-subunit interfaces of RBD, and/or elimination of one or more hydrophobic residues in the inter-subunit interfaces. Unless otherwise noted, the term inter-subunit interface of RBD as used herein refers to the residues of SARS-CoV-2 spike protein Receptor Binding Domain (RBD) that are in contact with or occluded by other parts of the trimer spike in the closed conformation, and are thus inaccessible to antibodies in live virus while being likely sources of aggregation for the RBD alone, expressed in the absence of the remainder of the spike protein. This term does not encompass RBD residues that interact with the host receptor ACE2 (the RBD-ACE2 interface). Examples of the inter-subunit interfaces include residues at the inter-subunit interfaces between 2 neighboring RBDs in the trimeric spike, inter-subunit interface with the NTD (aka S1_{A}), inter-subunit interface with the center of the spike, and inter-subunit interface of the with the S1_{B} hinge.

Using the wildtype RBD sequence (SEQ ID NO:2) of the Wuhan-Hu-1 isolate reported in Wu et al. (Nature 579: 265-269, 2020; NCBI Accession No. N_045512.2) as exemplification, N-linked glycans were engineered at these inter-subunit interfaces using the substitutions: A372T or A372S to introduce an N-linked glycan at N370, S383N/P384V to introduce a glycosylation at position 383 K386N/N388S or K386N/N388T to introduce an N-linked glycan at position 386, Y396T or Y396S to introduce an N-linked glycan at N394, D428N to introduce an N-linked glycan at position 428, and L517N/H519S or L517N/H519T to introduce an N-linked glycan at position 517 (FIG. 1B) and the mutations A520N/P521G/A522T or A520N/P521V/A522T. In addition, hydrophobic residues mutated at the inter-subunit interface that did not introduce an N-linked glycan include V367, L390, L518 (e.g., L518G), A520, and A522 (FIG. 1C).

In various embodiments, several specific mutations can be introduced into the inter-subunit interfaces to impart formation of novel glycosylation sites. These include, e.g., V362S, V362/T, L517N/H519T, L517N/H519S, A520N/P521X/A522(S/T) (X is any amino acid except for P), A372T, A372S, Y396T, D428N, R357N/S359T, R357N/S359S, S371N/S373T, S371N/S373S, S383N plus P384 mutated to a residue other than proline (e.g., S383N+P384V/A/I/L/M/W), K386N/N388T, K386N/N388S, and G413N. Typically, the engineered RBD polypeptides of the invention contain the noted substitutions at least one of these residues. In some embodiments, the engineered RBD polypeptides of the invention contain the noted substitutions at a combination of residues A372/Y396, A372/L517/H519, Y396/L517/H519, D428/L517/H519. In some of these embodiments, the engineered RBD polypeptides contain the noted substitutions at a combination of residues A372/Y396/L517/H519, A372/D428/L517/H519, and Y396/D428/L517/H519. In a specific embodiment, the engineered RBD polypeptide contains the noted substitutions at residues A372/Y396/D428/L517/H519, as exemplified herein with engineered RBD polypeptide “gRBD” (SEQ ID NO:3).

Complete S spike sequence, NCBI Sequence accession YP_009724390.1 (SEQ ID NO:1):

MFVFLVLLPL VSSQCVNLTT RTQLPPAYTN SFTRGVYYPD
KVFRSSVLHS TQDLFLPFFS NVTWFHAIHV SGTNGTKRFD
NPVLPFNDGV YFASTEKSNI IRGWIFGTTL DSKTQSLLIV
NNATNVVIKV CEFQFCNDPF LGVYYHKNNK SWMESEFRVY
SSANNCTFEY VSQPFLMDLE GKQGNFKNLR EFVFKNIDGY
FKIYSKHTPI NLVRDLPQGF SALEPLVDLP IGINITRFQT
LLALHRSYLT PGDSSSGWTA GAAAYYVGYL QPRTFLLKYN
ENGTITDAVD CALDPLSETK CTLKSFTVEK GIYQTSNFRV
QPTESIVRFP NITNLCPFGE VFNATRFASV YAWNRKRISN
CVADYSVLYN SASFSTFKCY GVSPTKLNDL CFTNVYADSF
VIRGDEVRQI APGQTGKIAD YNYKLPDDFT GCVIAWNSNN
LDSKVGGNYN YLYRLFRKSN LKPFERDIST EIYQAGSTPC
NGVEGENCYF PLQSYGFQPT NGVGYQPYRV VVLSFELLHA
PATVCGPKKS TNLVKNKCVN FNFNGLTGTG VLTESNKKFL
PFQQFGRDIA DTTDAVRDPQ TLEILDITPC SFGGVSVITP
GTNTSNQVAV LYQDVNCTEV PVAIHADQLT PTWRVYSTGS
NVFQTRAGCL IGAEHVNNSY ECDIPIGAGI CASYQTQTNS
PRRARSVASQ SIIAYTMSLG AENSVAYSNN SIAIPTNFTI
SVTTEILPVS MTKTSVDCTM YICGDSTECS NLLLQYGSFC
TQLNRALTGI AVEQDKNTQE VFAQVKQIYK TPPIKDFGGF
NFSQILPDPS KPSKRSFIED LLFNKVTLAD AGFIKQYGDC
LGDIAARDLI CAQKFNGLTV LPPLLTDEMI AQYTSALLAG
TITSGWTFGA GAALQIPFAM QMAYRFNGIG VTQNVLYENQ
KLIANQFNSA IGKIQDSLSS TASALGKLQD VVNQNAQALN
TLVKQLSSNF GAISSVLNDI LSRLDKVEAE VQIDRLITGR
LQSLQTYVTQ QLIRAAEIRA SANLAATKMS ECVLGQSKRV
DFCGKGYHLM SFPQSAPHGV VFLHVTYVPA QEKNFTTAPA
ICHDGKAHFP REGVFVSNGT HWFVTQRNFY EPQIITTDNT
FVSGNCDVVI GIVNNTVYDP LQPELDSFKE ELDKYFKNHT
SPDVDLGDIS GINASVVNIQ KEIDRLNEVA KNLNESLIDL
QELGKYEQYI KWPWYIWLGF IAGLIAIVMV TIMLCCMTSC
CSCLKGCCSC

Wild-type RBD sequence is a 197 aa (331-527) (SEQ ID NO:2), as shown below:

NITNLCPFGEVFNATRFASVYAWNRKRISNCVADYS [symbol] LYNSA
SFSTFKCYGVSPTKLNDLCFTNVYADSFVIRGDEVRQIAPGQT
GKIADYNYKLPDDFTGCVIAWNSNNLDSKVGGNYNYLYRLFRK
SNLKPFERDISTEIYQAGSTPCNGVEGENCYFPLQSYGFQPTN
GVGYQPYRVVVLSFELLHAPATVCGP

Engineered RBD variant gRBD (SEQ ID NO:3) is shown below. In the sequence, glycosylations sites are italicized, and mutated residues from the wild-type RBD are underlined.

NITNLCPFGEVFNATRFASVYAWNRKRISNCVADYSVLYNST
SFSTFKCYGVSPTKLNDLCFTNVTADSFVIRGDEVRQIAPGQ
TGKIADYNYKLPDNFTGCVIAWNSNNLDSKVGGNYNYLYRLF
RKSNLKPFERDISTEIYQAGSTPCNGVEGENCYFPLQSYGFQ
PTNGVGYQPYRVVVLSFENLTAPATVCGP

In addition or as alternative to the substitutions forming novel glycosylation sites, the engineered RBD polypeptides of the invention contain mutations that eliminate some hydrophobic residues at the RBD inter-subunit interfaces. As exemplified with the wildtype RBD sequence shown in SEQ ID NO:2, the hydrophobic residues to be mutated include, e.g., one or more residues selected from V362, V367, A372, L390, L455, L517, L518, A520, P521, or A522. In various embodiments, each of the residues to be mutated is substituted with a charged amino acid residue. In some of these embodiments, the substituting residue is Asp or Glu.

In some embodiments, the engineered RBD polypeptides of the invention contain one or more mutations that result in formation of novel glycosylation sites and also one or more additional substitutions that eliminate hydrophobic residues at the RBD inter-subunit interfaces, as noted above. In some of these embodiments, the engineered RBD contains substitution of residue L518 in addition to mutations that form two glycosylation sites. In some of these embodiments, the engineered RBD contain the following combinations of mutations relative to the wildtype RBD sequence: L517N/H519(T/S)+A372(T/S)+L518(D/E/G), L517N/H519(T/S)+Y396T/S+L518(D/E/G), D428N, L517N/H519(T/S)+D428N+L518(D/E/G), A372(T/S)+Y396T/S+L518(D/E/G), A372(T/S)+D428N+L518(D/E/G), Y396T/S+D428N+L518(D/E/G), A372(T/S)+Y396T/S+L517D/E, A372(T/S)+D428N+L517D/E, Y396T/S+D428N+L517D/E, A372(T/S)+Y396T/S+L517D/E+L518(D/E/G), A372(T/S)+D428N+L517D/E+L518(D/E/G), Y396T/S+D428N+L517D/E+L518(D/E/G), L517N/H519(T/S)+V372(D/E), and L517N/H519(T/S)+V372(D/E)+L390(D/E).

In addition to the exemplified RBD polypeptides herein, the engineered RBD polypeptides of the invention also encompass RBD variants that contain an amino acid sequence that is substantially identical to or conservatively modified variant of any of the exemplified RBD polypeptides, e.g., SEQ ID NO:3. Also, while the exemplified RBD polypeptide herein are derived from a specific SARS-CoV-2 isolate with full S protein sequence shown in SEQ ID NO:1, RBD sequences from other SARS-CoV-2 isolates can also be readily employed to produce engineered RBD immunogen polypeptides of the invention. Due to functional similarity and sequence homology among different isolates or strains the virus, engineered soluble RBD immunogens derived from other known S protein ortholog sequences can also be generated in accordance with the strategy described herein. There are many known coronavirus S protein sequences that have been described in the literature. The corresponding RBD sequences can be readily retrieved. See, e.g., James et al., J. Mol. Biol. 432:3309-25, 2020; Andersen et al., Nat. Med. 26:450-452, 2020; Walls et al., Cell 180:281-292, 2020; Zhang et al., J. Proteome Res. 19:1351-1360, 2020; Du et al., Expert Opin. Ther. Targets 21:131-143.; 2017; Yang et al., Viral Immunol. 27:543-550, 2014; Wang et al., Antiviral Res. 133:165-177, 2016; Bosch et al., J. Virol. 77:8801-8811, 2003; Lio et al., TRENDS Microbiol. 12:106-111, 2004; Chakraborti et al., Virol. J. 2:73, 2005; and Li, Ann. Rev. Virol. 3:237-261, 2016.

In addition to the various substitutions noted above, the engineered coronavirus RBD immunogen polypeptides of the invention can further contain a trimerization motif at the C-terminus. Suitable trimerization motifs for the invention include, e.g., T4 fibritin foldon (PDB ID: 4NCV) and viral capsid protein SHP (PDB: 1TD0). T4 fibritin (foldon) is well known in the art, and constitutes the C-terminal 30 amino acid residues of the trimeric protein fibritin from bacteriophage T4, and functions in promoting folding and trimerization of fibritin. See, e.g., Papanikolopoulou et al., J. Biol. Chem. 279: 8991-8998, 2004; and Guthe et al., J. Mol. Biol. 337: 905-915, 2004. Similarly, the SHP protein and its used as a functional trimerization motis are also well known in the art. See, e.g., Dreier et al., Proc Natl Acad Sci USA 110: E869-E877, 2013; and Hanzelmann et al., Structure 24: 140-147, 2016. An exemplary foldon sequences is GYIPEAPRDGQAYVRKDGEWVLLSTFL (SEQ ID NO:4). In some embodiments, the trimerization motif is linked to the engineered RBD immunogen polypeptide via a short GS linker. The inclusion of the linker is intended to stabilize the formed trimer molecule. In various embodiments, the linker can contain 1-6 tandem repeats of GS. In some embodiments, an His6-tag can be added to the C-terminus of the trimerization motif to facilitate protein purification, e.g., by using a Nickel column.

### IV. Scaffolded RBD Polypeptides and Related Vaccine Compositions

The invention provides a number of multimerization platforms to generate fusion proteins. These scaffold proteins can be used to multimerize various antigens, including the engineered RBD polypeptides described herein. In some embodiments, the invention provides vaccine compositions that are derived from the engineered RBD polypeptides. Typically, the vaccines of the invention contain or are capable of expressing the engineered RBD immunogens in multimeric forms as detailed herein. Vaccines containing or expressing the engineered RBD polypeptides described herein engineered RBD polypeptides described herein can be provided in various forms. These include, e.g., as expressed proteins that are fused to or displayed by a multimerization scaffold (e.g., a nanoparticle scaffold), as mRNA nanoparticles, as viral vectors, or as DNA-based vaccines.

The engineered RBD polypeptides of the invention can be conjugated or fused to a multimeric protein scaffold to form multimerized immunogens. In some embodiments, the engineered RBD polypeptide in the vaccines is provided as a trimeric molecule. This can be achieved by fusing the RBD polypeptide to a trimerization motif described above, e.g., foldon. More preferably, the RBD immunogen present in or expressed by the vaccines is a multimer of at least 10-mer, 12-mer, 24-mer or 60-mer. Compared to monomeric RBD or a trimeric derivative thereof, such multimerized immunogens are more suitable for eliciting antibody response in vaccine compositions. In some embodiments, the RBD immunogens present in or expressed by the vaccines can be 12-mer, 24-mer or 60-mer. In some embodiments, the engineered RBD immunogen can be conjugated to a heterologous protein scaffold. In some embodiments, the engineered RBD sequence can be fused to a heterologous scaffold to impart formation of a multimer. In some of these embodiments, the heterologous scaffold is a nanoparticle scaffold, e.g., a self-assembling nanoparticle.

In some embodiments, the vaccine compositions contain or are capable of expressing an engineered RBD polypeptide that is fused to a heterologous multimerization scaffold. Any multimerization protein scaffold can be used to present the engineered RBD immunogen protein or polypeptide in the construction of the vaccines of the invention. This includes a virus-like particle (VLP) such as bacteriophage Q_{β} VLP and nanoparticles. In some of these embodiments, a self-assembling nanoparticle scaffold can be used. In general, the nanoparticles employed in the invention need to be formed by multiple copies of a single subunit, e.g., 12, 24, or 60 subunits, and have 3-fold axes on the particle surface.

A number of well-known nanoparticle scaffolds can be employed in producing the vaccine compositions of the invention. These include, e.g., ferritin, I3-01 derived sequence (e.g., mi3), the HP-NAP/Dps family proteins, the DPSL family of proteins, the Dodecin family proteins, and half-ferritins/encapsulated ferritin proteins. Examples of these platform sequences are described herein (e.g., SEQ ID NOs:4-10). Any of these sequences, as well as conservatively modified variants or substantially identical sequences thereof, can all be employed in the practice of the invention. Depending on the specific nanoparticle or multimerization platform used, either the C-terminus or the N-terminus of the engineered coronavirus immunogen polypeptide can be fused to the subunit sequence of the multimerization scaffold. In some embodiments, a linker sequence (e.g., a GS linker) may be used to link the engineered coronavirus RBD polypeptide to the scaffold subunit sequence. Exemplary linker sequences include GGSGGGGSGPG (SEQ ID NO:23), GSSGSSGGSGGS (SEQ ID NO:24), GGGSGGTGG (SEQ ID NO:25), and GGGSGGGPGSG (SEQ ID NO:26).

In some embodiments, an I3-01 derived nanoparticle sequence is used to multimerize an engineered RBD polypeptide of the invention. I3-01 is an engineered protein that can self-assemble into hyperstable nanoparticles. See, e.g., Hsia et al., Nature 535, 136-139, 2016. This scaffold allows display of an immunogen in a 60-er format. Several modified sequences derived from I3-01 have been reported for vaccine development, including the mi3 scaffold exemplified herein. See, e.g., Bruun et al., ACS Nano. 12: 8855-66, 2018; and He et al., Sci Adv. 4: eaau6769, 2018. As exemplification, the subunit sequence of a mi3 60-mer scaffold (SEQ ID NO:5) is described herein for multimerization of an engineered RBD polypeptide of the invention, gRBD.

In some embodiments, the multimerization platform is ferritin. Ferritin is a globular protein found in all animals, bacteria, and plants. As is well known in the art, it acts primarily to control the rate and location of polynuclear Fe(III)₂O₃ formation through the transportation of hydrated iron ions and protons to and from a mineralized core. The globular form of ferritin is made up of monomeric subunit proteins (also referred to as monomeric ferritin subunits), which are polypeptides having a molecule weight of approximately 17-20 kDa. As exemplification, a specific 24-mer ferritin nanoparticle sequence (SEQ ID NO:5) is described herein for displaying the engineered RBD polypeptides of the invention. This Helicobacter pylon non-heme ferritin sequence was derived from NCBI Accession #WP_000949190 amino acids 5-167 with the mutations S21A and C31A.

In some other vaccine compositions of the invention, the protein scaffold for multimerization of the engineered RBD polypeptide can be one derived from the HP-NAP/Dps family proteins, the DPSL family of proteins or the Dodecin family proteins. HP-NAP is the Dps (DNA protection in starvation) protein of Helicobacter pylori. Dps proteins are similar to ferritin, but form 12mers. HP-NAP additionally has the property of being a TLR2 agonist and is thus self-adjuvanting, skewing toward a favorable anti-viral Th1 response, a possible advantage for a DNA vaccine. It also expressed very well on the Dps from Salmonella Enterica. The H. pylori NAP sequence exemplified herein (SEQ ID NO:7) was derived from NCBI Accession #WP_000846479. Use of Dps proteins as nanoparticle platforms can be carried out as described in the art, e.g., PCT publication WO2011082087.

In some other embodiments, the multimerization platform in the vaccines of the invention is derived from a member of the DPSL protein family. These proteins represent an evolutionary midway point between ferritins and the Dps family of proteins. Like Dps, it is comprised of a 12-mer, but has an enzymatic fold more closely related to ferritin. It is further distinguished from the Dps family in that it has a pair of cysteines which form a disulfide within a single monomer unit. As exemplification, a DPSL scaffold is described herein for fusion with the engineered RBD polypeptide of the invention. This protein sequence (SEQ ID NO:8) is derived from the bfr gene (bacterioferritin related protein) of Bacteroides fragilis, the genome of which also contains distinct ferritin (ftna) and Dps (dps) genes. This exemplified BfDPSL sequence corresponds to amino-acids 2-170 of accession #WP_005782541 with three further mutations, C136S eliminates an unpaired cysteine, and S112A eliminates a potential cryptic glycosylation site at N110. The BfDPSL protein has the advantage over the archaeal DPSLs of having a free external C-terminus for conjugation, and the potential to provide universal T-cell help.

In still some other embodiments, the multimerization protein scaffold used in the invention can be one derived from the Dodecin family proteins. Dodecins, which provide a 12-mer platform, have the advantage of a very short multimerization motif. A specific dodecin sequence (SEQ ID NO:9) derived from Bordelia Pertussis is exemplified herein. This B. Pertussis dodecin derived sequence corresponds to amino acids 2-71 of NCBI Accession #WP_010930433. Unlike the other platforms, both N and C-termini can be used for fusion with the immunogen polypeptide. In some preferred embodiments, the engineered RBD polypeptide is fused to C-terminus of the docecin sequence.

In still some other embodiments, an engineered RBD polypeptide of the invention can be multimerized by fusion to a half-ferritin/encapsulated ferritin protein. This family of proteins are another branch of the ferritin superfamily. They differ in structure from ferritin, Dps and DPSL oligomers in they are 10-mers arranged in a disc composed of five dimers, and they contain no interior space. In these proteins, the N-termini are buried at the center of the disk, and the free C-termini are located at the periphery. Though smaller and containing fewer subunits than Dps, these proteins have a similar hydrodynamic radius due to their radial distribution. As exemplified herein, a construct with the RBD polypeptide (gRBD) fused to a half-ferritin (SEQ ID NO:10) from Acidiferrobacteraceae bacterium expressed at a very high level with low aggregation. Relative to the wildtype sequence (NCBI accession #HEC13526), sequence of the half-ferritin platform exemplified herein contains a C44A substitution to eliminate an unpaired cysteine.

The half-ferritin of Acidiferrobacteraceae bacterium was selected, in part, because it is from a thermophile. The Acidiferrobacteraceae bacterium the half-ferritin sequence used as a scaffold herein (SEQ ID NO: 10) is from was isolated from sediment around a hydrothermal vent (Zhou et al., mSystems 2020 Jan. 7; 5(1):e00795-19). A scaffold protein that is a substantially identical or conservatively modified variant of a protein from a thermophile or hyperthermophile has the potential to exhibit the enhanced stability that is often observed for proteins from thermophiles.

Half-ferritins, such as the one derived from Acidiferrobacteraceae bacterium (SEQ ID NO:10), were designated “F10” proteins, because they are ferritin proteins comprised of 10 subunits. The number of subunits for this class of protein is confirmed by the crystal structure of the F10 protein of Nitrosomonas europaea (PDB ID: 3K6C). Such F10 proteins appear to be excellent vaccine antigen scaffolds.

Sequences of the subunits of the various nanoparticle or multimerization scaffolds described herein are all known in the art and/or exemplified herein. More detailed information on the structural and functional properties of the various nanoparticle scaffolds, as well as their use in presenting multimeric protein immunogens, is provided in the art. See, e.g., Bruun et al., ACS Nano. 12: 8855-66, 2018; Hsia et al., Nature 535, 136-139, 2016; He et al., Sci Adv. 4: eaau6769, 2018; Gauss et al., Biochemistry 45:10815-27, 2006; Gauss et al., J Bacteriol. 194: 15-27, 2012; Duan et al., Immunity 49: 301-311, 2018; Eggink et al., J. Virol. 88: 699-704, 2014; Jardine et al., Science 351: 1458-63, 2016; Kulp et al., Nat. Commun. 8: 1655, 2017; Trevino et al., J Mol Biol. 366:449-60, 2007; U.S. Pat. No. 7,608,268B2; and PCT publications WO2011082087, WO2017/192434, WO2019/089817, and WO2019/241483. In various embodiments, the coronavirus vaccine compositions of the invention can employ any of these known nanoparticles, as well as their conservatively modified variants or variants with substantially identical (e.g., at least 90%, 95% or 99% identical) sequences.

Subunit sequence of mi3 60-mer scaffold (SEQ ID NO:5)

MKMEELFKKHKIVAVLRANSVEEAKKKALAVFLGGVHLIEITF
TVPDADTVIKELSFLKEMGAIIGAGTVTSVEQARKAVESGAEF
IVSPHLDEEISQFAKEKGVFYMPGVMTPTELVKAMKLGHTILK
LFPGEVVGPQFVKAMKGPFPNVKFVPTGGVNLDNVCEWFKAGV
LAVGVGSALVKGTPVEVAEKAKAFVEKIRGCTE

Subunit sequence of ferritin (SEQ ID NO:6)

DIIKLLNEQVNKEMNSANLYMSMSSWAYTHSLDGAGLFLF
DHAAEEYEHAKKLIIFLNENNVPVQLTSISAPEHKFEGLT
QIFQKAYEHEQHISESINNIVDHAIKSKDHATFNFLQWYV
AEQHEEEVLFKDILDKIELIGNENHGLYLADQYVKGIAKS
RKS

Subunit sequence of NAP (SEQ ID NO:7)

MKTFEILKHLQADAIVLFMKVHNFHWNVKGTDFFNVHKAT
EEIYEGFADMFDDLAERIAQLGHHPLVTLSEALKLTRVKE
ETKTSFHSKDIFKEILEDYKHLEKEFKELSNTAEKEGDKV
TVTYADDQLAKLQKSIWMLQAHLA

Subunit sequence of BfDPSL (SEQ ID NO:8)

AKESVKILQGKLDVKSLIDQLNAALSEEWLAYYQYWVGAL
VVEGAMRADVQGEFEEHAEEERHHAQLIADRIIELEGVPV
LDPKKWFELARCKYDSPTAFDSVSLLNONVASERCAILRY
QEIANFINGKDYTTSDIAKHILAEEEEHEQDLQDYLTDIA
RMKESFLKK

Subunit sequence of dodecin (SEQ ID NO:9)

SSHVYKQIELVGSSAVSSDDAIAQAIARASDTLRHLDWFE
VTETRGHIKDGKVAHWQVSLKIGMRLEADD

Subunit sequence of Ap half-ferritin (SEQ ID NO:10)

MANEGYHEEISDLSDETRDMHRAIVSLMEELEAVDWYNQRV
DAAQDGDLKAILAHNRDEEKEHAAMVLEWIRRKDPAFDKEL
KDYLFTEKPIAHST

Sequences of gRBD-Fc and gRBD-foldon fusions, as well as several other specific nanoparticle displayed or scaffolded RBD immunogens are exemplified below. In the sequences, the gRBD sequence is shown underlined, a GS linker region is italicized, and the scaffold subunit sequence (e.g., mi3 60-mer scaffold) is shown italicized and underlined.

gRBD-Fc fusion
(SEQ ID NO: 11)
NITNLCPFGEVENATRFASVYAWNRKRISNCVADYSVLYNSTSFSTFKCYGVSP
TKLNDLCFTNVTADSFVIRGDEVRQIAPGQTGKIADYNYKLPDNFTGCVIAWNS
NNLDSKVGGNYNYLYRLFRKSNLKPFERDISTEIYQAGSTPCNGVEGENCYFPL
QSYGFQPTNGVGYQPYRVVVLSFENLTAPATVCGPGGSGGSDKTHTCPPCPAP
ELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK
gRBD-foldon fusion
(SEQ ID NO: 12)
NITNLCPFGEVENATRFASVYAWNRKRISNCVADYSVLYNSTSFSTF
KCYGVSPTKLNDLCFTNVTADSFVIRGDEVRQIAPGQTGKIADYNYKLPDNFTG
CVIAWNSNNLDSKVGGNYNYLYRLFRKSNLKPFERDISTEIYQAGSTPCNGVE
GENCYFPLQSYGFQPTNGVGYQPYRVVVLSFENLTAPATVCGPGGSGGGGSGP
GGYIPEAPRDGQAYVRKDGEWVLLSTEL
NAP-gRBD
(SEQ ID NO: 13):
MKTFEILKHLQADAIVLFMKVHNFHWNVKGTDFFNVHKATEEIYEGFADMFDDLA
ERIAQLGHHPLVTLSEALKLTRVKEETKTSFHSKDIFKEILEDYKHLEKEFKELS
NTAEKEGDKVTVTYADDQLAKLQKSIWMLQAHLAGGGSGGGPGSGNITNLCPFGE
VENATRFASVYAWNRKRISNCVADYSVLYNSTSFSTFKCYGVSPTKLNDLCFTNV
TADSFVIRGDEVRQIAPGQTGKIADYNYKLPDNFTGCVIAWNSNNLDSKVGGNYN
YLYRLFRKSNLKPFERDISTEIYQAGSTPCNGVEGENCYFPLQSYGFQPTNGVGY
QPYRVVVLSFENLTAPATVCGP
gRBD-ferritin
(SEQ ID NO: 14):
NITNLCPFGEVFNATRFASVYAWNRKRISNCVADYSVLYNSTSFSTFKCYGV
SPTKLNDLCFTNVTADSFVIRGDEVRQIAPGQTGKIADYNYKLPDNFTG
CVIAWNSNNLDSKVGGNYNYLYRLFRKSNLKPFERDISTEIYQAGSTPCNGVE
GFNCYFPLQSYGFQPTNGVGYQPYRVVVLSFENLTAPATVCGPGGGSGGTGGD
IIKLLNEQVNKEMNSANLYMSMSSWAYTHSLDGAGLFLFDHAAEEYEHAKKLI
IFLNENNVPVQLTSISAPEHKFEGLTQIFQKAYEHEQHISESINNIVDHAIKS
KDHATFNFLQWYVAEQHEEEVLEKDILDKIELIGNENHGLYLADQYVKGLAKS
RKS
gRBD-mi3 fusion
(SEQ ID NO: 15):
NITNLCPFGEVENATRFASVYAWNRKRISNCVADYSVLYNSTSFSTFKCYGVSP
TKLNDLCFTNVTADSFVIRGDEVRQIAPGQTGKIADYNYKLPDNFTGCVIAWNS
NNLDSKVGGNYNYLYRLFRKSNLKPFERDISTEIYQAGSTPCNGVEGENCYFPL
QSYGFQPTNGVGYQPYRVVVLSFENLTAPATVCGPGSSGSSGGSGGSMKMEELF
KKHKIVAVLRANSVEEAKKKALAVFLGGVHLIEITFTVPDADTVIKELSFLKEM
GAIIGAGTVTSVEQARKAVESGAEFIVSPHLDEEISQFAKEKGVFYMPGVMTPT
ELVKAMKLGHTILKLFPGEVVGPQFVKAMKGPFPNVKFVPTGGVNLDNVCEWFK
AGVLAVGVGSALVKGTPVEVAEKAKAFVEKIRGCTE
BfDPSL-gRBD fusion
(SEQ ID NO: 16):
AKESVKILQGKLDVKSLIDQLNAALSEEWLAYYQYWVGALVVEGAMRADVQGEF
EEHAEEERHHAQLIADRIIELEGVPVLDPKKWFELARCKYDSPTAFDSVSLLNQ
NVASERCAILRYQEIANFTNGKDYTTSDIAKHILAEEEEHEQDLQDYLTDIARM
KESFLKKGGGSGGGPGSGNITNLCPFGEVFNATRFASVYAWNRKRISNCVADYS
VLYNSTSFSTFKCYGVSPTKLNDLCFTNVTADSFVIRGDEVRQIAPGQTGKIAD
YNYKLPDNFTGCVIAWNSNNLDSKVGGNYNYLYRLFRKSNLKPFERDISTEIYQ
AGSTPCNGVEGFNCYFPLQSYGFQPTNGVGYQPYRVVVLSFENLTAPATVCGP
Ap half-ferritin-gRBD fusion
(SEQ ID NO: 17):
MANEGYHEEISDLSDETRDMHRAIVSLMEELEAVDWYNQRVDAAQDGDLKAILA
HNRDEEKEHAAMVLEWIRRKDPAFDKELKDYLFTEKPIAHSTGGGSGGGPGSGN
ITNLCPFGEVENATRFASVYAWNRKRISNCVADYSVLYNSTSFSTFKCYGVSPT
KLNDLCFTNVTADSFVIRGDEVRQIAPGQTGKIADYNYKLPDNFTGCVIAWNSN
NLDSKVGGNYNYLYRLFRKSNLKPFERDISTEIYQAGSTPCNGVEGENCYFPLQ
SYGFQPTNGVGYQPYRVVVLSFENLTAPATVCGP
BpDo-gRBD fusion
(SEQ ID NO: 18):
SSHVYKQIELVGSSAVSSDDALAQALARASDTLRHLDWFEVTETRGHIKDGKVAH
WQVSLKIGMRLEADDGGGSGGGPGSGNITNLCPFGEVENATRFASVYAWNRKRIS
NCVADYSVLYNSTSFSTFKCYGVSPTKLNDLCFTNVTADSFVIRGDEVRQIAPGQ
TGKIADYNYKLPDNFTGCVIAWNSNNLDSKVGGNYNYLYRLFRKSNLKPFERD
ISTEIYQAGSTPCNGVEGENCYFPLQSYGFQPTNGVGYQPYRVVVLSFENLTAP
ATVCGP
SaHisB-gRBD
(SEQ ID NO: 19)
MIYQKQRNTAETQLNISISDDQSPSHINTGVGFLNHMLTLFTFHSGLSLNIEAQG
DIDVDDHHVTEDIGIVIGQLLLEMIKDKKHFVRYGTMYIPMDETLARVVVDISGR
PYLSFNAALSKEKVGTEDTELVEEFFRAVVINARLTTHIDLIRGGNTHHEIEAIF
KAFSRALGIALTATDDQRVPSSKGVIEGGGSGGGPGSGNITNLCPFGEVENATRF
ASVYAWNRKRISNCVADYSVLYNSTSFSTFKCYGVSPTKLNDLCFTNVTADSFVI
RGDEVRQIAPGQTGKIADYNYKLPDNFTGCVIAWNSNNLDSKVGGNYNYLYRLFR
KSNLKPFERDISTEIYQAGSTPCNGVEGENCYFPLQSYGFQPTNGVGYQPYRVVV
LSFENLTAPATVCGP
SaClpP-gRBD
(SEQ ID NO: 20)
MNLIPTVIETTNRGERAYDIYSRLLKDRIIMLGSQIDDNVANSIVSQLLFLQAQ
DSEKDIYLYINSPGGSVTAGFAIYDTIQHIKPDVQTIAIGMAASMGSFLLAAGA
KGKRFALPNAEVMIHQPLGGAQGQATEIEIAANHIRKTREKLNRILSERTGQSI
EKIQKDTDRDNELTAEEAKEYGLIDEVMVPETKLEGGGSGGGPGSGNITNLCPF
GEVENATRFASVYAWNRKRISNCVADYSVLYNSTSFSTFKCYGVSPTKLNDLCF
TNVTADSFVIRGDEVRQIAPGQTGKIADYNYKLPDNFTGCVIAWNSNNLDSKVG
GNYNYLYRLFRKSNLKPFERDISTEIYQAGSTPCNGVEGENCYFPLQSYGFQPT
NGVGYQPYRVVVLSFENLTAPATVCGP
AbEncFtn-gRBD
(SEQ ID NO: 21)
MANEGYHEEISDLSDETRDMHRAIVSLMEELEAVDWYNQRVDAAQDGDLKAIL
AHNRDEEKEHAAMVLEWIRRKDPAFDKELKDYLFTEKPIAHSTGGGSGGGPGS
GNITNLCPFGEVENATRFASVYAWNRKRISNCVADYSVLYNSTSFSTFKCYGV
SPTKLNDLCFTNVTADSFVIRGDEVRQIAPGQTGKIADYNYKLPDNFTGCVIA
WNSNNLDSKVGGNYNYLYRLFRKSNLKPFERDISTEIYQAGSTPCNGVEGENC
YFPLQSYGFQPTNGVGYQPYRVVVLSFENLTAPATVCGP
gRBD-fntFrt
(SEQ ID NO: 22)
NITNLCPFGEVFNATRFASVYAWNRKRISNCVADYSVLYNSTSFSTFKCYGVSP
TKLNDLCFTNVTADSFVIRGDEVRQIAPGQTGKIADYNYKLPDNFTGCVIAWNS
NNLDSKVGGNYNYLYRLFRKSNLKPFERDISTEIYQAGSTPCNGVEGENCYFPL
QSYGFQPTNGVGYQPYRVVVLSFENLTAPATVCGPGGGSGGTGGMLSKDIIKLL
NEQVNKEMNSANLYMSMSSWAYTHSLDGAGLFLFDHAAEEYEHAKKLIIFLNEN
NVPVQLTSISAPEHKFEGLTQIFQKAYEHEQHISESINNIVDHAIKSKDHATEN
FLQWYVAEQHEEEVLFKDILDKIELIGNENHGLYLADQYVKGIAKSRKS

Scaffolded RBD vaccine compositions of the invention encompass any of these fusion sequences, as well as substantially identical or conservatively modified variant sequences thereof. Other than the displayed RBD polypeptide and the scaffold sequence, the sequence of a nanoparticle vaccine composition of the invention can include additional motifs for better biological or pharmaceutical properties. In some embodiments, the fusion constructs can contain a N-terminal leader sequence as described herein, e.g., MKHLWFFLLLVAAPRWVLS (SEQ ID NO:27). Some additional structural components in the constructs can function to facilitate the immunogen display on the surface of the nanoparticles, to enhance the stability of the displayed immunogens, to facilitate purification of expressed proteins, and/or to improve yield and purity of the self-assembled protein vaccines. In some of these embodiments, a N-terminal epitope tag can be inserted to facilitate expression and purification of the recombinant protein. For example, the exemplified gRBD-ferritin fusion shown in SEQ ID NO:14 or the gRBD-fntFrt fusion (SEQ ID NO:22) can include a N-terminal FLAG tag, DYKDDDDK (SEQ ID NO:28), which can be fused to gRBD via a linker motif, e.g., GGGP (SEQ ID NO:29). In some other embodiments, a C-tag, EPEA (SEQ ID NO:30) or a combination of SnoopTag and C-tag, KLGSIEFIKVNKGSGEPEA (SEQ ID NO:31) can be added at the C-terminus of the multimerized RBD constructs of the invention. For example, the C-tag can be fused via a linker motif, e.g., GSGGG (SEQ ID NO:32) at the C-terminus in the exemplified fusion constructs shown in SEQ ID NOs:12, 13 and 16-21. As additional exemplification, the SnoopTag and C-tag combination can be fused via a linker motif, e.g., GGSG (SEQ ID NO:33) to the C-terminus of the exemplified gRBD-mi3 construct shown in SEQ ID NO:15. In still some other embodiments, rather than either a C-Tag or a FLAG-tag, a polyhistidine tag can be used in the multimerized RBD constructs to facilitate production of the protein vaccines.

In some other embodiments, a protein ligation system such as SnoopCatcher/SnoopTag or SpyCatcher/SpyTag may be included in the scaffolded RBD polypeptide of the invention. In these embodiments, an engineered RBD sequence (e.g., SEQ ID NO:3) can be fused to a SnoopTag or a SpyTag motif, and the scaffold sequence (e.g., a nanoparticle subunit sequence) can be fused to a SnoopCatcher or a SpyCatcher motif. Alternatively, the RBD sequence can be fused to a SnoopCatcher or a SpyCatcher motif, and the scaffold sequence can be fused to a SnoopTag or a SpyTag motif. As exemplification, a SnoopCatcher or a SpyCatcher can be attached to the C-terminus of one of the multimerization scaffolds described herein (e.g., mi3, HisB, ClpP, or EncFrt), and a corresponding Tag motif can be fused to an engineered RBD sequence or another polypeptide sequence. Upon introducing the two constructs expressing the Tag fusion and the Catcher fusion into host or producer cells, vaccines presenting the engineered RBD polypeptide (or another polypeptide of interest) can be produced as a result of the Tag/Catcher mediated ligation of the RBD polypeptide (or another polypeptide of interest) to the multimerization scaffold sequence.

### V. Scaffold Proteins for Displaying Antigens in General

The invention provides scaffold proteins that can be used for multimerizing any antigens or immunogen polypeptides in general, as well as fusion proteins thus generated. As exemplified herein with gRBD multimerized by scaffold proteins Staphylococcus aureus HisB (SaHisB) or Staphylococcus aureus ClpP (SaClpP) (SEQ ID NO:19 or 20), the antigens are typically fused to the C-terminus of these scaffold proteins. These scaffold proteins allow efficient expression of the fusion proteins and are able to maintain proper biological and immunogenic properties of the fused antigens. In addition to fusions that contain an engineered RBD polypeptide as exemplified herein, the various multimerization platforms or scaffold proteins described herein (e.g., HisB and ClpP) are suitable for constructing fusions with any other antigens or immunogenic polypeptides of interest. Any type of antigen or immunogen polypeptides can be fused to one of the scaffold proteins described herein. In some embodiments, the employed antigens are immunogen polypeptides from pathogens such as infectious bacteria, virus, fungi or parasites. In some embodiments, the employed antigens are tumor antigens, for example, tumor antigens for metastatic epithelial cancer, colorectal carcinoma, gastric carcinoma, oral carcinoma, pancreatic carcinoma, ovarian carcinoma, or renal cell carcinoma. In some other embodiments, the employed antigens are human proteins whose expression levels or compositions have been correlated with human disease or other phenotype. Examples of such antigens include adhesion proteins, hormones, growth factors, cellular receptors, autoantigens, autoantibodies, and amyloid deposits.

In general, the scaffold protein for generating fusion with any given antigen should possess one or more of the following properties. It should have an available C-terminus for proper folding and assembly. It needs to be larger than 9 nm to enhance immunogenicity. It should have a multimericity lower than about 60, e.g., from about 13 to about 59. This is because expression decreases at higher multimericity without an increase in immunogenicity. In some embodiments, the scaffold protein should require no coordination by cysteine. This is because proper folding of some bacterial proteins is dependent upon cysteine residues that coordinate metal ions in a reducing environment of a bacterial cell. Such protein would not be suitable for the fusions of the invention because of the oxidizing environment of the secretory pathway or extracellular environment in mammals. Additionally, the chosen scaffold protein should also not be one that binds to nucleic acids, including bacterial, viral, and phage proteins that self-assemble around nucleic acids (e.g., viral capsid proteins). In some embodiments, the employed scaffold protein should also not be a membrane protein or a toxin. In some embodiments, the employed scaffold protein should also not be a homopolymer. This is to avoid many layers of complexity associated with coordinated expression of multiple proteins. In some embodiments, the employed scaffold protein possesses all these properties.

In some embodiments, the employed scaffold protein to display an antigen of interest is from a human pathogen or vaccine strain. For instance, in certain embodiments the scaffold protein is from, e.g., Staphylococcus aureus, Mycobacterium tuberculosis, Mycobacterium bovis, Pseudomonas aeruginosa, Pseudomonas oryzihabitans, Bordetella pertussis, Bacillus anthracis, Neisseria meningitidis, Clostridioides difficile, or Candida albicans.

In certain embodiments, the scaffold protein is from a commensal bacterium. For instance, in certain embodiments the scaffold protein is from, e.g., Staphylococcus epidermidis, Escherichia coli, Bifidobacterium bifidum, Lactobacillus casei, Parasutterella excrementihominis, or Cutibacterium avidum.

In certain embodiments, the scaffold protein is from a thermophile or hyperthermophile. For instance, in certain embodiments the scaffold is from, e.g., Thermus aquaticus, Thermus thermophilus, Thermus scotoductus, Thermus oshiami, Thermus parvatiensis, Thermus atranikianii, Marinithermus hydrothermalis, Ardenticatenales bacterium, Moorella humiferra, Moorela thermoacetica, Thermoanaerobacterium thermosaccharolyticum, Geobacillus thermoglucosidasius, Pyrococcus furiosus, Petrotoga halophila, Thermococcus chitonophagus, Thermococcus gammatolerans, Thermococcus kodakarensis, Thermococcus barossii, Thermococcus piezophilus, Thermococcus thioreducens, Thermococcus celer, Thermococcus barophilus, Thermococcus paralvinellae, Thermococcus cleftensis, Thermococus radiotolerans, Thermococcus sibiricus, Paleococcus pacificus, Pyrodictium delaneyi, Pyrodictium occultum, Methanosarcina thermophila, or Chaetomium thermophilum.

In certain embodiments, the scaffold protein is a consensus sequence derived from several phylogenetically-related species, e.g., a Staphylococcus consensus, a Bacillus consensus, a Pseudomonas consensus, a Pyrococcus consensus, a Moorella consensus, a Pyrodictium consensus, a Thermus consensus, a Thermococcus consensus, or a Candida consensus.

In certain embodiments, the scaffold protein lacks a cysteine amino acid residue. The scaffold may lack a cysteine residue due to the engineering of the sequence to remove a wild-type cysteine residue. Alternatively, the wild-type protein sequence of the scaffold may lack a cysteine residue. Notably, the optimal scaffold protein does not include a metal ion that is coordinated by cysteine residues.

In certain embodiments, the scaffold protein does not bind nucleic acids. Certain multimerization domains bind nucleic acids or depend upon binding nucleic acids. However, binding of nucleic acid is, in certain embodiments, not necessary for multimerization.

In certain embodiments, the scaffold protein is an imidazoleglycerol-phosphate dehydratase (HisB) protein. HisB is a protein that presents idealized features as a scaffold protein. These that HisB is a self-assembling homo-multimer of more than 12 but less than 60 subunits. Specifically, HisB is a homo-multimer of 24 subunits. Importantly, HisB also contains a C-terminus that is exposed at the surface of the homo-multimer, and the C-terminus is amenable to fusions with vaccine antigens, e.g., SARS-CoV-2 RBD vaccine antigens. Indeed, the fusion protein constructed from the HisB protein of Staphylococcus aureus and the gRBD vaccine antigen (SaHisB-gRBD, SEQ ID NO: 19) expressed efficiently.

Scaffold sequences based on HisB can be derived from human pathogens, human commensals, and other mesophilic bacteria, including, e.g.:

Staphylococcus aureus HisB
(SEQ ID NO: 34)
MIYQKQRNTAETQLNISISDDQSPSHINTGVGFLNHMLTL
FTFHSGLSLNIEAQGDIDVDDHHVTEDIGIVIGQLLLEMI
KDKKHFVRYGTMYIPMDETLARVVVDISGRPYLSFNAALS
KEKVGTFDTELVEEFFRAVVINARLTTHIDLIRGGNTHHE
IEAIFKAFSRALGIALTATDDQRVPSSKGVIE
Staphylococcus epidermidis HisB
(SEQ ID NO: 35)
MNYQIKRNTEETQLNISLANNGTQSHINTGVGFLDHMLTL
FTFHSGLTLSIEATGDTYVDDHHITEDIGIVIGQLLLELV
KTQQSFTRYGCSYVPMDETLARTVVDISGRPYFSFNSKLS
AQKVGTFDTELVEEFFRALVINARLTVHIDLLRGGNTHHE
IEAIFKSFARALKISLAQNEDGRIPSSKGVIE
Escherichia coli HisB
(SEQ ID NO: 36)
MSQKYLFIDRDGTLISEPPSDFQVDRFDKLAFEPGVIPEL
LKLQKAGYKLVMITNQDGLGTQSFPQADFDGPHNLMMQIF
TSQGVQFDEVLICPHLPADECDCRKPKVKLVERYLAEQAM
DRANSYVIGDRATDIQLAENMGITGLRYDRETLNWPMIGE
QLTRRDRYAHVVRNTKETQIDVQVWLDREGGSKINTGVGF
FDHMLDQIATHGGFRMEINVKGDLYIDDHHTVEDTGLALG
EALKIALGDKRGICRFGFVLPMDECLARCALDISGRPHLE
YKAEFTYQRVGDLSTEMIEHFFRSLSYTMGVTLHLKTKGK
NDHHRVESLFKAFGRTLRQAIRVEGDTLPSSKGVL
Mycobacterium tuberculosis HisB
(SEQ ID NO: 37)
MTTTQTAKASRRARIERRTRESDIVIELDLDGTGQVAVDT
GVPFYDHMLTALGSHASFDLTVRATGDVEIEAHHTIEDTA
IALGTALGQALGDKRGIRRFGDAFIPMDETLAHAAVDLSG
RPYCVHTGEPDHLQHTTIAGSSVPYHTVINRHVFESLAAN
ARIALHVRVLYGRDPHHITEAQYKAVARALRQAVEPDPRV
SGVPSTKGAL
Mycobacterium bovis HisB
(SEQ ID NO: 38)
MTTTQTAKASRRARIERRTRESDIVIELDLDGTGQVAVDT
GVPFYDHMLTALGSHASFDLTVRATGDVEIEAHHTIEDTA
IALGTALGQALGDKRGIRRFGDAFIPMDETLAHAAVDLSG
RPYCVHTGEPDHLQHTTIAGSSVPYHTVINRHVFESLAAN
ARIALHVRVLYGRDPHHITEAQYKAVARALRQAVEPDPRV
SGVPSTKGAL
Pseudomonas aeruginosa HisB
(SEQ ID NO: 39)
MAERKASVARDTLETQIKVSIDLDGTGKARFDTGVPFLDH
MMDQIARHGLIDLDIECKGDLHIDDHHTVEDIGITLGQAF
AKAIGDKKGIRRYGHAYVPLDEALSRVVIDFSGRPGLQMH
VPFTRASVGGFDVDLFMEFFQGFVNHAQVTLHIDNLRGHN
THHQIETVFKAFGRALRMAIELDERMAGQMPSTKGCL
Pseudomonas oryzihabitans HisB
(SEQ ID NO: 40)
MAERKATVERNTLETQVKVSLDLDGTGAARFDTGVPFLEH
MLDQIARHGLIDLDIHCRGDLHIDDHHTVEDIGITLGQAF
AKAVGDKKGIQRYGHAYVPLDEALSRVVIDFSGRPGLHWN
VPFTRATVGRMDVDLFLEFFQGFTNHAQVTLHVDNLRGVN
SHHQIETVFKAFGRALRMALAEDPRMAGVMPSTKGCL
Bordetella pertussis HisB
(SEQ ID NO: 41)
MRTAEITRNTNETRIRVAVNLDGTGKQTIDTGVPFLDHML
DQIARHGLIDLDIKADGDLHIDAHHTVEDVGITLGMAIAK
AVGSKAGLRRYGHAYVPLDEALSRVVIDFSGRPGLEYHID
FTRARIGDFDVDLTREFFQGLVNHALMTLHIDNLRGFNAH
HQCETVFKAFGRALRMALEVDPRMGDAVPSTKGVL
Bifidobacterium bifidum HisB
(SEQ ID NO: 42)
MARTAHIVRETSESHIELSLNLDGTGKTDIDTSVPFYNHM
MNALGKHSLIDLTIHAHGDTDIDVHHTVEDTAIVFGEALK
QALGDKRGIRRFADATVPLDEALAKAVVDISGRPYCVCSG
EPDGFEYCMIGGHFTGSLVRHVMESIAFHAGICLHMQVLA
GRDPHHIAEAEFKALARALRFAVEPDPRIQGLIPSTKGAL
Lactobacillus casei HisB
(SEQ ID NO: 43)
MRTATITRTTKETQITISLNLDQQSGIAIDTGIGFFDHML
EAFAKHGRFGLTIKAQGDLDVDPHHTIEDTGIVLGSCFKQ
ALGDKAGIERFGSAFVPMDETLARVVVDLSGRAYLVFAAE
LTNQRLGGFDTEVTEDFFQAVAFAGEFNLHAAVLYGRNTH
HKIEALFKALGRSMQAAVSENPAVKGIPSTKGVI
Bacillus subtilis HisB
(SEQ ID NO: 44)
MRKAERVRKTNETDIELAFTIDGGGQADIKTDVPFMTHML
DLFTKHGQFDLSINAKGDVDIDDHHTTEDIGICLGQALLE
ALGDKKGIKRYGSAFVPMDEALAQVVIDLSNRPHLEMRAD
FPAAKVGTFDTELVHEFLWKLALEARMNLHVIVHYGTNTH
HMIEAVFKALGRALDEAATIDPRVKGIPSTKGML
Bacillus anthracis HisB
(SEQ ID NO: 45)
MRESSQIRETTETKIKLSLQLDEGKNVSVQTGVGFFDHML
TLFARHGRFGLQVEAEGDVFVDAHHTVEDVGIVLGNCLKE
ALQNKEGINRYGSAYVPMDESLGFVAIDISGRSYIVFQGE
LTNPKLGDFDTELTEEFFRAVAHAANITLHARILYGSNTH
HKIEALFKAFGRALREAVERNAHITGVNSTKGML
Parasutterella excrementihominis His B
(SEQ ID NO: 46)
MTRRADVKRQTAETSILVSMDLDGTGKADIRTGIGFFDHM
LHQIARHGQIDLTVMCDGDLHIDGHHSVEDIGIAMGQCLA
KALGDKAGITRFGSAYVPLDEALSRTVLDISGRPYLVWNV
DFTAAMIGEFDTQLPREFFLALADNARITLHIDNLRGINA
HHQCESVFKSFGRALRMACEYDPRARNVIPSTKGVL
Streptococcus mutans HisB
(SEQ ID NO: 47)
MRQAKIERNTFETKIKLSLNLDTQEPVDIQTGVGFFDHML
TLFARHGRMSLVVKADGDLHVDSHHTVEDVGIALGQALRQ
ALGDKVGINRYGTSFVPMDETLGMASLDLSGRSYLVFDAE
FDNPKLGNFDTELVEEFFQALAFNVQMNLHLKILHGKNNH
HKAESLFKATGRALREAVTINPEIKGVNSTKGML
Streptococcus sanguinis HisB
(SEQ ID NO: 48)
MRQAEIKRKTQETDIELAVNLDQQEPVAIETGVGFFDHML
TLFARHSRISLTVKAEGDLWVDSHHTVEDVGIVLGQALRQ
ALGDKAGINRYGTSFVPMDETLGMASLDLSGRSYLVFEAD
FDNPKLGNFDTELVEEFFQALAFNLQMNLHLKILHGKNSH
HKAESLFKATGRALREAITINPEIHGVNSTKGLL
Cutibacterium avidum HisB
(SEQ ID NO: 49)
MTHRCAHVHRETSESNVDVSIDLDGEGESTISTGVGFYDH
MLTALAKHSGIDMSITTTGDVEIDGHHSVEDTAIVLGQAL
AQALGDKRGIARFGDAVVPLDEALAQCVVDVAGRPWVECT
GEPEGQIYARLGGSGVPYQGSMTYHVVQSLALNAGLCVHL
RLLAGRDPHHICEAQYKALARALRIAVAPDPRNAGRVPST
KGALDV
Neisseria meningitidis HisB
(SEQ ID NO: 50)
MAKLEKHTGKPKGWLDRKHRERTVPETAAESTGTAETQIA
ETASAAGCRSVTVNRNTCETQITVSINLDGSGKSRLDTGV
PFLEHMIDQIARHGMIDIDISCKGDLHIDDHHTAEDIGIT
LGQAIRQALGDKKGIRRYGHSYVPLDEALSRVVIDLSGRP
GLVYNIEFTRALIGRFDVDLFEEFFHGIVNHSMMTLHIDN
LSGKNAHHQAETVFKAFGRALRMAVEHDPRMAGQTPSTKG
TLTA
Corynebacterium glutamicum HisB
(SEQ ID NO: 51)
MTVAPRIGTATRTTSESDITVEINLDGTGKVDIDTGLPFF
DHMLTAFGVHGSFDLKVHAKGDIEIDAHHTVEDTAIVLGQ
ALLDAIGEKKGIRRFASCQLPMDEALVESVVDISGRPYFV
ISGEPDHMITSVIGGHYATVINEHFFETLALNSRITLHVI
CHYGRDPHHITEAEYKAVARALRGAVEMDPRQTGIPSTKG
AL
Clostridioides difficile HisB
(SEQ ID NO: 52)
MRIWKVERNTLETQILVELNIDGSGKAEIDTGIGFLDHML
TLMSFHGKFDLKVICKGDTYVDDHHSVEDIGIAIGEAFKN
ALGDKKGIRRYSNIYIPMDESLSMVAIDISNRPYLVFNAK
FDTQMIGSMSTQCFKEFFRAFVNESRVTLHINLLYGENDH
HKIESIFKAFARALKEGSEIVSNEIASSKGVL
Clostridium acetobutylicum HisB
(SEQ ID NO: 53)
MEEKRTAFIERKTTETSIEVDINLDGEGKYDIDTGIGFFD
HMLELMSKHGLIDLKVKVIGDLKVDSHHTVEDTGIVIGEC
INKALGNKKSINRYGTSFVPMDESLCQVSMDISGRAFLVF
DGEFTCEKLGDFQTEMVEEFFRALAFNAGITLHARVIYGK
NNHHMIEGLFKAFGRALSEAVSKNTRIKGVMSTKGSI
Ochrobactrum anthropic HisB
(SEQ ID NO: 54)
MTAESTRKASIERSTKETSIAVSVDLDGVGKFDITTGVGF
FDHMLEQLSRHSLIDMRVMAKGDLHIDDHHTVEDTGIALG
QAIAKALGERRGIVRYASMDLAMDDTLTGAAVDVSGRAFL
VWNVNFTTSKIGTFDTELVREFFQAFAMNAGITLHINNHY
GANNHHIAESIFKAVARVLRTALETDPRQKDAIPSTKGSL
KG
Rhodococcus ruber HisB
(SEQ ID NO: 55)
MSEQTTPTPRTARIERTTKESSIVVELNLDGTGRTDIATG
VPFYDHMLTALGQHASFDLTVRAQGDIEIEAHHTVEDTAI
VLGQALNQALGDKRGIRRFGDAFIPMDETLAHAAVDVSGR
PYCVHTGEPDYMVHSVIGGYPGVPYSTVINKHVFESLAFH
ARIALHVRVLYGRDQHHITEAEFKAVARALRQAVEPDPRV
SGVPSTKGTL
Streptomyces venezuelae HisB
(SEQ ID NO: 56)
MSRVGRVERTTKETSVVVEIDLDGTGKVDVSTGVGFYDHM
LDQLGRHGLFDLTVKTDGDLHIDSHHTIEDTALALGAAFK
QALGDKVGIYRFGNCTVPLDESLAQVTVDLSGRPYLVHTE
PENMAPMIGSYDTTMTRHIFESFVAQAQIALHIHVPYGRN
AHHIVECQFKAFARALRYASERDPRAAGILPSTKGAL
Sinorhizobium medicae HisB
(SEQ ID NO: 57)
MADVTPSRTGQVSRKTNETAVSVALDVEGTGSSKIVTGVG
FFDHMLDQLSRHSLIDMDIKAEGDLHVDDHHTVEDTGIAI
GQALAKALGDRRGITRYASIDLAMDETMTRAAVDVSGRPF
LVWNVAFTAPKIGTFDTELVREFFQALAQHAGITLHVQNI
YGANNHHIAETCFKSVARVLRTATEIDPRQAGRVPSTKGT
LA

The HisB proteins from certain thermophiles and hyperthermophiles may be advantageous, due to the stability requirements for enzymes that are functional at comparatively high temperatures. Scaffold proteins can be derived from the HisB of thermophilic and hyperthermophilic bacteria, including, e.g., any one of the following:

Thermus aquaticus HisB
(SEQ ID NO: 58)
MREALVERATAETWVRLRLGLDGPVGGKVATGLPFLDHML
LQLQRHGRFLLEVEARGDLEVDVHHLVEDVGITLGMALKE
ALGEGAGLERYAEAFAPMDETLVLCVLDLSGRPHLEYRPE
AWPVVGEAGGVNHYHLREFLRGLVNHGRLTLHLKLLSGRE
AHHVLEASFKALARALHRATRLTGEGLPSTKGVL
Thermus thermophilus HisB
(SEQ ID NO: 59)
MREATVERATAETWVWLRLGLDGPTGGKVDTGLPFLDHML
LQLQRHGRFLLEVEARGDLEVDVHHLVEDVGIALGMALKE
ALGDGVGLERYAEAFAPMDETLVLCVLDLSGRPHLEFRPE
AWPVVGEAGGVNHYHLREFLRGLVNHGRLTLHLRLLSGRE
AHHVVEASFKALARALHKATRRTGEGVPSTKGVL
Thermus scotoductus HisB
(SEQ ID NO: 60)
MREASVERATAETWVRVRLGLDGPPGGKVATGLPFLDHML
LQLQRHGRFLLEVEARGDLEVDVHHLVEDVGITLGQALRE
ALGEGRGVERYAEAFAPMDETLVLCVLDLSGRPHLEYRPE
EWPVVGEAGGVNHYHLREFLRGLVNHGRLTLHLRLLSGRE
AHHVVEASFKALARALHRATRITGEELPSTKGVL
Thermus oshimai
(SEQ ID NO: 61)
MREALVERATAETWVKVRLGLDGPVGGEVATGLPFLDHML
LQLQRHGRFLLEVSAKGDLEVDVHHLVEDVGITLGLALKE
ALGEGRGLERYGEAYAPMDETLVLCVLDLSGRPHLEFRPE
DWPVEGAAGGMNHYHLREFLRGLANHGRLTLHLRLLSGRE
AHHVLEASFKALARALHRATRLTGEGLPSTKGVL
Thermus parvatiensis
(SEQ ID NO: 62)
MREALVERATAETWVRLRLGLDGPTGGKVDTGLPFLDHML
LQLQRHGRFLLEVEARGDLEVDVHHLVEDVGIALGMALKE
ALGEGVGLERYAEAFAPMDETLVLCVLDLSGRPHLEYRPE
EWPVVGEAGGVNHYHLREFLRGLVNHGRLTLHLRLLSGRE
AHHVVEASFKALARALHRATRRTGEGVPSTKGVL
Thermus antranikianii
(SEQ ID NO: 63)
MREASVERATAETWVRVRLGLDGPPGGKVATGLPFLDHML
LQLQRHGRFLLEVEAKGDLEVDVHHLVEDVGITLGQALRE
ALGEGRGVERYAEAFAPMDETLVLCVLDLSGRPHLEYRPE
EWPVVGEAGGVNHYHLREFLRGLVNHGRLTLHLRLLSGRE
AHHVVEASFKALARALHRATRITGEELPSTKGVL
Marinithermus hydrothermalis
(SEQ ID NO: 64)
MRNARIVRHTTETQVQLELGLDGPVGGEVRTGLPFLDHML
LQLQRHGRFHLEVRAQGDLEVDVHHLVEDVGITLGQAVKQ
AVGDARGIERYADAFAPMDETLVHVVLDVSGRPHLAFEPE
RLEVVGAPGGVNVFHLREFLRGLVNHAGLTLHLRVLAGRE
AHHVIEASFKALARALFQATRLTRADLPSTKEVL

Consensus sequence of Thermus HisB proteins, where “X” is any amino acid that is present at that same position in a Thermus HisB protein (SEQ ID NO:65):

Thermus HisB protein
(SEQ ID NO: 65):
MREAXVERATAETWVRLRLGLDGPXGGKVATGLPFLDHML
LQLQRHGRFLLEVEARGDLEVDVHHLVEDVGITLGMALKE
ALGEGRGLERYAEAFAPMDETLVLCVLDLSGRPHLEYRPE
EWPVVGEAGGVNHYHLREFLRGLVNHGRLTLHLRLLSGRE
AHHVVEASFKALARALHRATRLTGEGLPSTKGVL
Ardenticatenales bacterium HisB
(SEQ ID NO: 66)
MPESSSSAPTRRAVINRSTNETRIQLSLFLDGSGGGTRQT
GVPFLDHMLDHVARHGLLDLEIKAAGDYEIDDHHTVEDVG
IVLGKALSEALGNKAGIRRYGDATVPMDEALVLCAVDFSG
RGLLAFQGTIPTPKVGTFDTELVAEFLRALASNGGMTLHI
QVLAGQNSHHIIEGIFKALGRALREAVEIDERRGGAVPST
KGMLE
Moorella humiferrea HisB
(SEQ ID NO: 67)
MNREALIERRTAETCIRVKLDLDGSGKWQGSSGIPFFDHL
LAQLARHGLLDLEIQAEGDLEVDNHHTIEDIGICLGQAVK
QALGDKAGINRYGHTLIPMDEALVQVVLDLSGRPYLAYNL
DLAPGRIGSLETELLEEFLRAFVNHGALTLHVQKLAGRNG
HHIAEALFKALGRAIREAASRDPRVEGIPSTKGNLV
Moorella thermoacetica HisB
(SEQ ID NO: 68)
MSREALIERQTTETNIRLKVDLDGSGTWQGSSGIPFFDHL
LGQMARHGLLDLKVWAEGDLEVDNHHTVEDIGICLGQAVK
KALGDKKGISRYGSALVPMDEALVL
VALDFSGRPYLAWGLELPPGRIGSLETELVEEFLRAMVNN
SGLTLHVRQLAGHNAHHLAEALFKALGRAIRQAVTLDPRV
QGIPSTKGSLS
Thermoanaerobacterium thermosaccharolyticum
HisB
(SEQ ID NO: 69)
MREAEVNRKTAETEVYVKINIDGAGKSHINTGIGFLDHML
NLFSKHGLFDLQVEAKGDLYVDSHHTVEDIGITLGQAFLK
ALGDKKSIKRYGLSYVPMDEALIRAVVDISGRPYLYYDLE
LKMQVLGNFETETVEDFFRAFAYNSYITLHIEQLHGKNTH
HIIEAAFKALGRSLDEATKIDDRIEGVPSTKGVL
Geobacillus thermoglucosidasius HisB
(SEQ ID NO: 70)
MAREAMIARTTNETSIQLSLSLDGEGKAELETGVPFLTHM
LDLFAKHGQFDLHIEAKGDTHIDDHHTTEDIGICLGQAIK
EALGDKKGIKRYGNAFVPMDDALAQVVIDLSNRPHFEFRG
EFPAAKVGAFDVELVHEFLWKLALEARMNLHVIVHYGRNT
HHMVEAVFKALGRALDEATMIDPRVKGVPSTKGML

In certain embodiments, diverse HisB sequences are utilized, e.g., as a prime and boost that do not include shared epitopes in the scaffold protein. A diverse source of HisB proteins is found in Archaea, including, e.g., Halobacterium salinarum HisB having the following sequence (SEQ ID NO:71):

MTDRTAAVTRETAETDVAVTLDLDGDGEHTVDTGIGFFDH
MLAAFAKHGLFDVTVRCDGDLDVDDHHTVEDVGIALGAAF
SEAVGEKRGIQRFADRRVPLDEAVASVVVDVSGRAVYEFD
GGFSQPTVGGLTSRMAAHFWRTFATHAAVTLHCGVDGENA
HHEIEALFKGVGRAVDDATRIDQRRAGETPSTKGDL

The HisB proteins from certain thermophile and hyperthermophile Archaea may be advantageous, due to the stability requirements for enzymes that are functional at comparatively high temperatures, and/or sequence diversity. Scaffold proteins can be derived from the HisB of thermophilic and hyperthermophilic Archaea, including, e.g., any of the following proteins:

Pyrococcus furiosus HisB
(SEQ ID NO: 72)
MRRTTKETDIIVEIGKKGEIKTNDLILDHMLTAFAFYLGK
DMRITATYDLRHHLWEDIGITLGEALRENLPEKFTRFGNA
IMPMDDALVLVSVDISNRPYANVDVNIKDAEEGFAVSLLK
EFVWGLARGLRATIHIKQLSGENAHHIVEAAFKGLGMALR
VATKESERVESTKGVL
Petrotoga halophila HisB
(SEQ ID NO: 73)
MRRKTNETDIEINYSTELFVDTGDLVLNHLLKTLFYYMEK
NVIIKAKFDLSHHLWEDMGITIGQFLRNEVEGKNIKRFGT
SILPMDDALILVSVDISRSYANIDINIKDTEKGFELGNFK
ELIMGLSRYLQSTIHIKQINGENAHHIIEASFKALGNALK
TALEVSEKHESTNKVYKL
Thermococcus chitonophagus HisB
(SEQ ID NO: 74)
MRRKTKETDIIVEIGKEGTIRTGDRVLDHMLTALFFYMGV
KASVKAEYDLRHHLWEDVGITLGEEIRAKLPEKFARFGNA
VMPMDDALVLVAVDISGRPYLSLELDPREGEEGFEVSLVR
EFLWGLVRSLRATIHVKQFSGINAHHIIEATFKGLGKALG
EAIKEVERLESTKGVI
Thermococcus gammatolerans HisB
(SEQ ID NO: 75)
MKRETRETSVEVELDAPFGVETGDRILDHMLTALFHYMGR
SARVKADYDLRHHLWEDVGITLGEELRSKLPEKFRRFGSA
ITPMDDALVLIAVDISGRPYVSAELSFEEGEEGFEKALVR
EFLWGLARSLKATIHVKTLSGTNAHHVIEATFKGLGIALA
QATRESERLESTKGLLEV
Thermococcus kodakarensis HisB
(SEQ ID NO: 76)
MRRTTKETDIEVELDVEGTVETGDPVLNHLLMALFHYMGR
NARVKANYDLRHHLWEDVGITLGLELREKLPGKFARFGSA
VMPMDDALILVALDISGRPYLNLELFPLEEEEGFSVTLVR
EFLWGLARSLRATIHVKQLGGVNAHHIIEAAFKGLGIALA
QAIAESERLESTKGVLE
Palaeococcus pacificus HisB
(SEQ ID NO: 77)
MRRKTRETDITVELGSEGGIKTGDKVFDHLLTALFFYMRE
EVSVSAEWDLRHHLWEDLGIVLGEELREKIKGRKIARFGN
AIIPMDDALVLVAVDISRPYLNLELAPDEGEEGFELTLVR
EFLWALARTLNATIHVKQLSGVNAHHVIEAAFKGLGVALR
KALRESERLESTKGVL
Thermococcus barossii HisB
(SEQ ID NO: 78)
MRRKTKETDVTVELDSKGSIRTGDKVLDHLLTALFFYMGR
EAKVEATYDLRHHLWEDVGITLGEELREKIPEKFTRFGNA
VMPMDDALVVVAVDISGRPYVNLELSFEEEEEGFEKTLVR
EFLWGLARSLKATVHVKTLSGVNAHHVIEAAFKGLGVALG
KAIQESGKLESTKGLLEV
Thermococcus piezophilus HisB
(SEQ ID NO: 79)
MRRKTKETDIIVEIGVEGGIETGDRVFDHLLTALFFYMRE
KANVKASYDLRHHLWEDLGITLGEELRDKIRGKKIARFGS
AIMPMDDALVLVAVDISRPYLNLEIDFKESEEGFKVTLVR
EFLRALARTLNATIHVKQLAGVNAHHIVEATFKGLGVALR
QALSEGERLESTKGVL
Thermococcus thioreducens HisB
(SEQ ID NO: 80)
MKRKTRETDVTVELDVAGEIRTGDGVLDHLLTALFFYMGR
EANVKASYDLRHHLWEDVGIVLGEELRSKLPERFARFGNA
AMPMDDALVLVVVDISGRPYVSAELTFEESEEGFEVSLVR
EFLWGLARSLKATIHVKTLSGVNAHHVIEAAFKGLGVALG
RAIQESGKLESTKGLLEV
Thermococcus celer HisB
(SEQ ID NO: 81)
MRRETGETEVTVELDVAGGIRTGDGVLDHLLTALFFYMGR
EARVEASYDLRHHLWEDVGITLGGELRGKLPERFARFGNA
VMPMDDALVLVAVDVSGRPYAAVELSFEEGEEGFEKALVR
EFLWGLARGLKATIHVKTLSGTNAHHVIEAAFKGLGVALG
KAVRESGKVESTKGLLEVWD
Thermococcus barophilus HisB
(SEQ ID NO: 82)
MRRKTKETDIIVEIGVDGGIETGDRVFDHLLTALFFYMQQ
NVSIKASYDLRHHLWEDLGIVLGEELREKIKGRKIARFGS
AIMPMDDALVLVAVDISRPYLNLELDIKESEKGFEVTLVR
EFLWALARTLNATIHMKQLAGVNAHHIIEAAFKGLGVALR
QALSESERLESTKGVL
Thermococcus paralvinellae HisB
(SEQ ID NO: 83)
MRRKTKETDIIVEIGVEGGIETGDRVFDHLLTALFFYMQQ
NVSIKASYDLRHHLWEDLGIVLGEELREKIKGRKIARFGS
AIMPMDDALVLVAVDISRPYLNLELDVKESEEGFEVTLVR
EFLWALARTLNATIHVKQLAGMNAHHIIEAAFKGLGVALR
QALRESKRLESTKGVL
Thermococcus cleftensis HisB
(SEQ ID NO: 84)
MRRTTRETDVTVELDSEGGIGTGDRVLDHLLTALFFYMGR
EAKVEATYDLRHHLWEDVGITLGEELRSKLPGKFARLGSA
VMPMDDALVVVAVDISGRPYVSLELSFEEEEEGFEKALVR
EFLWGLARSLKATVHVKTLSGVNAHHVIEAAFKGLGVALG
KAVRESGKLESTKGLLEV
Thermococcus radiotolerans HisB
(SEQ ID NO: 85)
MNRKTRETDVTVELDAAGGILTGDKVLDHLLTALFFYMGR
EAKVRASYDLRHHLWEDVGITLGEELRSKLPERFARFGSA
IMPMDDAFVLVAVDISGRPYASVELSFEEGEEGFEKALVR
EFLWGLARSLKATIHVKTLSGVNAHHVIEAAFKGLGAALG
KAIGESGKLESTKGLLEV
Thermococcus sibiricus HisB
(SEQ ID NO: 86)
MKRKTKETDITVEIDVNGSIETGDRIFNHLLTALFFYLHE
KVNIKASYDLRHHLWEDLGIVLGEELREKIKGKKIARFGS
AIIPMDDALVLVAVDISRPYLNLELDIKESEEGFEVTLVR
EFLWALARTLNATIHVKQLSGVNAHHIIEAAFKGLGVVLR
QALSESERLESTKGVL

Consensus sequence of Thermococcus HisB proteins, where “X” is any amino acid that is present at that same position in a

Thermococcus HisB protein
(SEQ ID NO: 87)
MRRKTKETDITVELDVEGGIETGDRVLDHLLTALFFYMGR
EAXVKASYDLRHHLWEDVGITLGEELREKLPGKFXRFGXA
VMPMDDALVVVAVDISGRPYLNLELXFEEXEEGFEVTLVR
EFLWGLARSLKATIHVKQLSGVNAHHVIEAAFKGLGVALX
QAIRESERLESTKGVLEXXX
Pyrodictium delaneyi HisB
(SEQ ID NO: 88)
MARRVKVERRTKETIVRVDVDLDGSELREIGVSTSVPFLD
HMVETLAYYAGWGLRVEVEEVKRVDDHHVAEDLALALGEA
IAKAVAAGGYRVARFGYAVVPMDEALVLVSVDYSGRPGAW
VELPLRRESIGGLATENIPHFMQSLAAAAGMTLHVVTLRG
ENDHHVAEAAFKALGMALRQALAQSQGVVSTKGAILPPRS
Pyrodictium occultum HisB
(SEQ ID NO: 89)
MARRARVERVTGETRVLVDLDLDARELRGVSVSTGVPFLD
HMVETLAYYAGWGLEARVEEAKRVDDHHVAEDLALALGEA
VARAVASGGYRVARFGHAIVPMDEVLVLAAVDYSGRPGAW
VDLPFTREEVGGLATENIPHFVWSLASASAMTVHVRALQG
GNNHHLAEAAFKALGMALRQALAPSAAVVSTKGVILPPGA
GARGGAGEE
Methanosarcina thermophila HisB
(SEQ ID NO: 90)
MRTGRMSRKTKETDIQLELNLDGTGIADVNTGIGFFDHML
ISFAKHAEFDLKVHADGDLYVDEHHLIEDTAIVLGKVLAD
ALGDMTGIARFGEARIPMDEALAEVALDIGGRSYLVLNAE
FSAPQVGQFSTQLVKHFFEALASNAKITIHASVYGDNDHH
KIEALFKAFAYAMKRAVKVEGKEVKSTKGLL

In addition to prokaryotic HisB proteins, HisB proteins from fungi can be used as scaffold proteins, including, e.g., any of the following proteins:

Saccharomyces cerevisiae HisB
(SEQ ID NO: 91)
MTEQKALVKRITNETKIQIAISLKGGPLAIEHSIFPEKEA
EAVAEQATQSQVINVHTGIGFLDHMIHALAKHSGWSLIVE
CIGDLHIDDHHTTEDCGIALGQAFKEALGAVRGVKRFGSG
FAPLDEALSRAVVDLSNRPYAVVELGLQREKVGDLSCEMI
PHFLESFAEASRITLHVDCLRGKNDHHRSESAFKALAVAI
REATSPNGTNDVPSTKGVLM
Schizosaccharomyces pombe HisB
(SEQ ID NO: 92)
MRRAFVERNTNETKISVAIALDKAPLPEESNFIDELITSK
HANQKGEQVIQVDTGIGFLDHMYHALAKHAGWSLRLYSRG
DLIIDDHHTAEDTAIALGIAFKQAMGNFAGVKRFGHAYCP
LDEALSRSVVDLSGRPYAVIDLGLKREKVGELSCEMIPHL
LYSFSVAAGITLHVTCLYGSNDHHRAESAFKSLAVAMRAA
TSLTGSSEVPSTKGVL
Candida tropicalis HisB
(SEQ ID NO: 93)
MSRQALINRITNETKIQIAINLDGGKLELKESIFPNKSVE
EEHAKQVSGGQYINVQTGIGFLDHMIHALAKHSGWSLIVE
CIGDLHIDDHHTAEDVGISLGMAFKEALGQIKGVKRFGSG
FAPLDEALSRAVVDLSNRPFAVIELGLKREKIGDLSTEMI
PHVLESFAGSAHITIHVDCLRGFNDHHRAESAFKALAIAI
KEAISKTGKDDVPSTKGVLY
Candida albicans HisB
(SEQ ID NO: 94)
MSREALINRITNETKIQIALNLDGGKLELKESIFPNQSII
IDEHHAKQVSGSQYINVQTGIGFLDHMIHALAKHSGWSLI
VECIGDLHIDDHHTAEDVGISLGMAFKQALGQIKGVKRFG
HGFAPLDEALSRAVVDLSNRPFAVIELGLKREKIGDLSTE
MIPHVLESFAGAAGITIHVDCLRGFNDHHRAESAFKALAI
AIKEAISKTGKNDIPSTKGVLS

In certain embodiments, HisB proteins from fungi that are thermophiles may be advantageous, due to the stability requirements for enzymes that are functional at comparatively high temperatures. Scaffold proteins can be derived from the HisB of thermophilic fungi, including, e.g., any of the following proteins:

Chaetomium thermophilum HisB
(SEQ ID NO: 95)
MSSQQNAPRWAAFARDTNETKIQVAINLDGGSFPPETDPR
LQVDSATEGHASQSTKSQTIKINTGIGFLDHMLHALAKHA
GWSLALACKGDLWIDDHHTAEDVCISLGYAFAKALGTPTG
LARFGSAYAPLDEALSRAVVDLSNRPYAVVDLGLRREKIG
DLSTEMLPHCLQSFAQAARITLHVDCLRGDNDHHRAESAF
KALAVALRQATSKVAGREGEVPSTKGTLSV
Thermothelomyces thermophilus HisB
(SEQ ID NO: 96)
MSSSQPAPRWAAFARDTNETKIQIALNLDGGAFPPDTDPR
LQVGDAGGHAAQSSKSQTITINTGIGFLDHMLHALAKHAG
WSLALACKGDLHIDDHHTAEDVCISLGYAFARALGTPTGL
ARFGSAYAPLDEALSRAVVDLSNRPYCVANLGLKREKIGD
LSTEMIPHCLHSFAGAARITLHVDCLRGDNDHHRAESAFK
ALAVAIRQATSRVAGREGEVPSTKGTLSV

In certain embodiments, the scaffold protein is the ATP-dependent Clp protease proteolytic subunit (ClpP). In certain embodiments, the ClpP protein sequence has one or both of the substitutions C92A and L144R (according to the position numbering of Staphylococcus aureus ClpP, SEQ ID NO:97), which knock out ATPase and protease activity. The absence of ATPase activity may reduce the energetic cost on the producing cell, thereby increasing antigen and scaffold production. ClpP presents certain optimal features for a scaffold protein. ClpP is self-assembling homo-multimer containing 14 subunits (i.e., a 14-mer). Importantly, the C-terminus of ClpP is exposed at the surface of the homo-multimer, allowing the fusion of protein antigens to its C-terminus. Indeed, the exemplified fusion of the gRBD vaccine antigen to ClpP (ClpP-gRBD; SEQ ID NO:20) expressed efficiently and assembled as a multimer. Suitable ClpP scaffold proteins may be derived from any of the sequences below:

Staphylococcus aureus ClpP
(SEQ ID NO: 97)
MNLIPTVIETTNRGERAYDIYSRLLKDRIIMLGSQIDDNV
ANSIVSQLLFLQAQDSEKDIYLYINSPGGSVTAGFAIYDT
IQHIKPDVQTICIGMAASMGSFLLAAGAKGKRFALPNAEV
MIHQPLGGAQGQATEIEIAANHILKTREKLNRILSERTGQ
SIEKIQKDTDRDNFLTAEEAKEYGLIDEVMVPETK
Staphylococcus epidermidis ClpP
(SEQ ID NO: 98)
MNLIPTVIETTNRGERAYDIYSRLLKDRIIMLGSQIDDNV
ANSIVSQLLFLQAQDSEKDIYLYINSPGGSVTAGFAIYDT
IQHIKPDVQTICIGMAASMGSFLLAAGAKGKRFALPNAEV
MIHQPLGGAQGQATEIEIAANHILKTREKLNRILSERTGQ
SIEKIQQDTDRDNFLTAAEAKEYGLIDEVMEPEK
Escherichia coli ClpP
(SEQ ID NO: 99)
MSYSGERDNFAPHMALVPMVIEQTSRGERSFDIYSRLLKE
RVIFLTGQVEDHMANLIVAQMLFLEAENPEKDIYLYINSP
GGVITAGMSIYDTMQFIKPDVSTICMGQAASMGAFLLTAG
AKGKRFCLPNSRVMIHQPLGGYQGQATDIEIHAREILKVK
GRMNELMALHTGQSLEQIERDTERDRFLSAPEAVEYGLVD
SILTHRN
Mycobacterium bovis ClpP
(SEQ ID NO: 100)
MSQVTDMRSNSQGLSLTDSVYERLLSERIIFLGSEVNDEI
ANRLCAQILLLAAEDASKDISLYINSPGGSISAGMAIYDT
MVLAPCDIATYAMGMAASMGEFLLAAGTKGKRYALPHAR
ILMHQPLGGVTGSAADIAIQAEQFAVIKKEMFRLNAEFTG
QPIERIEADSDRDRWFTAAEALEYGFVDHIITRAHVNGEA
Q
Pseudomonas aeruginosa ClpP
(SEQ ID NO: 101)
MSRNSFIPHVPDIQAAGGLVPMVVEQSARGERAYDIYSRL
LKERIIFLVGQVEDYMANLVVAQLLFLEAENPEKDIHLYI
NSPGGSVTAGMSIYDTMQFIKPNVSTTCIGQACSMGALLL
AGGAAGKRYCLPHSRMMIHQPLGGFQGQASDIEIHAKEIL
FIKERLNQILAHHTGQPLDVIARDTDRDRFMSGDEAVKYG
LIDKVMTQRDLAV
Pseudomonas oryzihabitans
(SEQ ID NO: 102)
MSRNSYMQSMPDIQAAGGLVPMVVEQSARGERAYDIYSRL
LKERVIFLVGQVEDYMANLVVAQLLFLEAENPDKDIHLYI
NSPGGSVTAGMSIYDTMQFIKPDVSTICIGQACSMGALLL
AGGAAEKRFCLPHSRMMIHQPLGGFQGQASDIEIHAREIL
TIRERLNKVLAHHTGQPMDVIARDTDRDNFMSGPEAVAYG
LIDKVLEKRNIPA
Bordetella pertussis ClpP
(SEQ ID NO: 103)
MQRFTDFYAAMHGGSSVTPTGLGYIPMVIEQSGRGERAYD
IYSRLLRERLIFLVGPVNDNTANLVVAQLLFLESENPDKD
ISFYINSPGGSVYAGMAIYDTMQFIKPDVSTLCTGLAASM
GAFLLAAGKKGKRFTLPNSRIMIHQPSGGAQGQASDIQIQ
AREILDLRERLNRILAENTGQPVERIAVDTERDNFMSAED
AVSYGLVDKVLTSRAQT
Bifidobacterium bifidum ClpP
(SEQ ID NO: 104)
MASEEAQFAARADRLAGPRGVVGFMPAAARESALRGGAAV
SPQNRYVLPQFSEKTPYGMKTQDPYTKLFEDRIIFMGVQV
DDTSADDIMAQLLVLESQDPSRDVMMYINSPGGSMTAMTA
IYDTMQYIKPDVQTVCLGQAASAAAILLAAGAKGKRLMLP
NARVLIHQPAIDQGFGKATEIEIQAKEMLRMREWLENTLA
KHTGQDVEKIRKDIEVDTFLTAQEAKDYGIVDEVLEHRS
Lactobacillus casei ClpP
(SEQ ID NO: 105)
MLVPTVVEQTSRGERAYDIYSRLLKDRIIMLSGEVNDQMA
NSVIAQLLFLDAQDSEKDIYLYINSPGGVITSGLAMLDTM
NFIKSDVQTIAIGMAASMASVLLAGGTKGKRFALPNSTIL
IHQPSGGAQGQQTEIEIAAEEILKTRKKMNQILADATGQT
VEQIKKDTERDHYMSAQEAKDYGLIDDILVNKNNQK
Bacillus subtilis ClpP
(SEQ ID NO: 106)
MNLIPTVIEQTNRGERAYDIYSRLLKDRIIMLGSAIDDNV
ANSIVSQLLFLAAEDPEKEISLYINSPGGSITAGMAIYDT
MQFIKPKVSTICIGMAASMGAFLLAAGEKGKRYALPNSEV
MIHQPLGGAQGQATEIEIAAKRILLLRDKLNKVLAERTGQ
PLEVIERDTDRDNFKSAEEALEYGLIDKILTHTEDKK
Bacillus anthracis ClpP
(SEQ ID NO: 107)
MNAIPYVVEQTKLGERSYDIYSRLLKDRIVIIGSEINDQV
ASSVVAQLLFLEAEDAEKDIFLYINSPGGSTTAGFAILDT
MNLIKPDVQTLCMGFAASFGALLLLSGAKGKRFALPNSEI
MIHQPLGGAQGQATEIEITAKRILKLKHDINKMIAEKTGQ
PIERVAHDTERDYFMTAEEAKAYGIVDDVVTKK
Parasutterella excrementihominis ClpP
(SEQ ID NO: 108)
MPDFSNFNSALIPMVIEQSGRGERSFDIYSRLLRDRVVFL
VGPVTDQSANLVVAQLLFLESENPDKDISLYIDSPGGSVY
AGLSIYDTMQFIKPDVSTICLGMAASMGAFLLAAGAKGKR
FALPNSRIMIHQPSGGTNGTAADIEIQAKEILELRSRLNT
ILSEHTGQSIEKIAVDTERDNFMSSAQAVEYGIIDGVFRK
RSEQIIKKK
Streptococcus mutans ClpP
(SEQ ID NO: 109)
MIPVVIEQTSRGERSYDIYSRLLKDRIIMLTGPVEDNMAN
SIIAQLLFLDAQDNTKDIYLYINSPGGSVSAGLAIVDTMN
FIKSDVQTIVMGIAASMGTIVASSGAKGKRFMLPNAEYLI
HQPMGGTGGGTQQSDMAIAAEQLLKTRKKLEKILSDNSGK
TIKQIHKDAERDYWMDAKETLKYGFIDEIMENNELK
Streptococcus sanguinis ClpP
(SEQ ID NO: 110)
MIPVVIEQTSRGERSYDIYSRLLKDRIIMLTGPVEDNMAN
SVIAQLLFLDAQDNTKDIYLYVNTPGGSVSAGLAIVDTMN
FIKSDVQTIVMGVAASMGTIIASSGAKGKRFMLPNAEYLI
HQPMGGAGSGTQQTDMAIVAEHLLRTRNTLEKILAENSGK
SVEQIHKDAERDYWMSAQETLEYGFIDEIMENSNLS
Cutibacterium avidum ClpP
(SEQ ID NO: 111)
MGFNAFDRSRLAALNAEQAEQAAPGGLAPASPRNDYYIPQ
WEERTSYGVRRVDPYTKLFEDRIIFLGTPVTDDIANAVMA
QLLCLQSMDADRQISMYINSPGGSFTAMTAIYDTMNYVRP
DVQTICLGMAASAAAVLLAAGAKGQRLSLPNSTILIHQPA
MGQATYGQATDIEILDDEIQRIRKLMEGMLADATGQSVEQ
VSKDIDRDKYLTAQGAKEYGLIDDVLTSL
Neisseria meningitidis ClpP
(SEQ ID NO: 112)
MSFDNYLVPTVIEQSGRGERAFDIYSRLLKERIVFLVGPV
TDESANLVVAQLLFLESENPDKDIFFYINSPGGSVTAGMS
IYDTMNFIKPDVSTLCLGQAASMGAFLLSAGEKGKRFALP
NSRIMIHQPLISGGLGGQASDIEIHARELLKIKEKLNRLM
AKHCGRDLADLERDTDRDNFMSAEEAKEYGLIDQVLENRA
SLQF
Corynebacterium glutamicum ClpP
(SEQ ID NO: 113)
MSNGFQMPTSRYVLPSFIEQSAYGTKETNPYAKLFEERII
FLGTQVDDTSANDIMAQLLVLEGMDPDRDITLYINSPGGS
FTALMAIYDTMQYVRPDVQTVCLGQAASAAAVLLAAGAPG
KRAVLPNSRVLIHQPATQGTQGQVSDLEIQAAEIERMRRL
METTLAEHTGKTAEQIRIDTDRDKILTAEEALEYGIVDQV
FDYRKLKR
Clostridioides difficile ClpP
(SEQ ID NO: 114)
MALVPVVVEQTGRGERSYDIFSRLLKDRIIFLGDQVNDAT
AGLIVAQLLFLEAEDPDKDIHLYINSPGGSITSGMAIYDT
MQYIKPDVSTICIGMAASMGAFLLAAGAKGKRLALPNSEI
MIHQPLGGAQGQATDIEIHAKRILKIKETLNEILSERTGQ
PLEKIKMDTERDNFMSALEAKEYGLIDEVFTKRP
Clostridium acetobutylicum ClpP
(SEQ ID NO: 115)
MSLVPYVIEQTSRGERSYDIYSRLLKDRVIFLGEEVNDTT
ASLVVAQLLFLESEDPDKDIYLYINSPGGSITSGMAIYDT
MQYVKPDVSTICIGMAASMGSFLLTAGAPGKRFALPNSEI
MIHQPLGGFKGQATDIGIHAQRILEIKKKLNSIYSERTGK
PIEVIEKDTDRDHFLSAEEAKEYGLIDEVITKH
Ochrobactrum anthropi ClpP
(SEQ ID NO: 116)
MRDPIETVMNLVPMVVEQTNRGERAYDIFSRLLKERIIFV
NGPVEDGMSMLVCAQLLFLEAENPKKEINMYINSPGGVVT
SGMAIYDTMQFIRPPVSTLCMGQAASMGSLLLTAGATGQR
YALPNARIMVHQPSGGFQGQASDIERHAQDIIKMKRRLNE
IYVKHTGRDYETIERTLDRDHFMTAQEALEFGLIDKVVES
RDVGADESK
Rhodococcus ruber ClpP
(SEQ ID NO: 117)
MTNLFDPRQLGGQAAAAPGGTAPASPASRYILPSFIEHSS
YGVKESNPYNKLFEERIIFLGVQVDDASANDVMAQLLVLE
SLDPDRDITMYINSPGGSFTSLMAIYDTMQYVRADITTVC
LGQAASAAAVLLAAGTPGKRLALPNARVLIHQPATGGIQG
QVSDLEIQAAEIERMRRLMETTLAKHTGKDPDQIRKDTDR
DKILTAAEAVDYGLIDNVLEYRKLSAQK
Streptomyces venezuelae ClpP
(SEQ ID NO: 118)
MVNTQMQNNFSASGLYTGPQVDNRYVIPRFVERTSQGVRE
YDPYAKLFEERVIFLGVQIDDASANDVMAQLLCLESMDPD
RDISIYINSPGGSFTALTAIYDTMQFVKPDIQTVCMGQAA
SAAAVLLAAGTPGKRMALPNARVLIHQPSGGTGREQLSDL
EIAANEILRMRDQLETMLAKHSTTPIEKIRDDIERDKILT
AEDALAYGLIDQIVSTRKNSH
Sinorhizobium medicae ClpP
(SEQ ID NO: 119)
MRNPVDTAMALVPMVVEQTNRGERSYDIYSRLLKERIIFL
TGPVEDHMATLVCAQLLFLEAENPKKEIALYINSPGGVVT
AGMAIYDTMQFIKPAVSTLCIGQAASMGSLLLAAGHKDMR
FATPNSRIMVHQPSGGFQGQASDIERHARDILKMKRRLNE
VYVKHCGRTYEEVEQTLDRDHFMSSDEALDWGLIDKVITS
RDAVEGME
Serratia marcescens ClpP
(SEQ ID NO: 120)
MEMDFKMHNDLGLGFICKNARTSSKPTLRKVTFPVSAYET
SKLSLTGFQCPTACRFPFFVLCMIIHNHLSSACPINQNEC
SNHISQFSIDIKVQDWLSRSRVAFIDFHNLRNTDKTTLIT
VEHLEALLTVMSTTLVAYAPYSKKRLNFSFLNSFTLSKTS
QSYTLTFPVVLSPLLDALGGFIQECITEKLLKRRNSNFMV
YEYLKRSGQSSHKVEDINNDLQLKTLNIRLMSVLTGLSQQ
GLISFICEGKRGDRRIEELQFIPYVQRTHPEVLTFQEWIS
PVD
Enterococcus faecalis ClpP
(SEQ ID NO: 121)
MNLIPTVIEQSSRGERAYDIYSRLLKDRIIMLSGPIDDNV
ANSVIAQLLFLDAQDSEKDIYLYINSPGGSVSAGLAIFDT
MNFVKADVQTIVLGMAASMGSFLLTAGQKGKRFALPNAEI
MIHQPLGGAQGQATEIEIAARHILDTRQRLNSILAERTGQ
PIEVIERDTDRDNYMTAEQAKEYGLIDEVMENSSALN

The ClpP proteins from certain thermophiles and hyperthermophiles may be advantageous, due to the stability requirements for enzymes that are functional at comparatively high temperatures. Scaffold proteins can be derived from the ClpP of thermophilic and hyperthermophilic bacteria, including, e.g., any of the following proteins:

Thermus aquaticus ClpP
(SEQ ID NO: 122)
MVIPYVIEQTARGERVYDIYSRLLKDRIIFLGTPIDAQVA
NTIVAQLLFLDAQNPNQEIRLYINSPGGEVDAGLAIYDTM
QFVRAPVSTIVIGMAASMAAVILAAGEKGRRYALPHSKVM
IHQPWGGARGTASDIAIQAQEILKAKKLLNEILAKHTGQP
LEKVERDTDRDYYLSAQEALEYGLIDQVVTREEA
Thermus thermophilus ClpP
(SEQ ID NO: 123)
MVIPYVIEQTARGERVYDIYSRLLKDRIIFLGTPIDAQVA
NVVVAQLLFLDAQNPNQEIKLYINSPGGEVDAGLAIYDTM
QFVRAPVSTIVIGMAASMAAVILAAGEKGRRYALPHAKIM
IHQPWGGVRGTASDIAIQAQEILKAKKLLNEILAKHTGQP
LEKVEKDTDRDYYLSAQEALEYGLIDQVVTREEA
Thermus scotoductus ClpP
(SEQ ID NO: 124)
MVIPYVIEQTARGERVYDIYSRLLKDRIIFLGTPIDSQVA
NIIVAQLLFLDAQNPNQEIRLYINSPGGEVDAGLAIYDTM
QFVRAPVSTIVIGMAASMAAVILAAGEKGRRYALPHSKVM
IHQPWGGVRGTASDIAIQAQEILKAKKLLNEILAKHTGQP
LEKVEKDTDRDYYLSAQEAMEYGLIDQVVTREEA
Thermus oshimai ClpP
(SEQ ID NO: 125)
MVIPYVIEQTARGERVYDIYSRLLKDRIIFLGTPIDAQVA
NTVVAQLLFLDAQNPNQEIRLYINSPGGEVDAGLAIYDTM
QFVRAPVSTIVIGMAASMAAVILAAGEKGRRYALPHAKV
MIHQPWGGARGTASDIAIQAQEILKAKKLLNEILAKHTGQ
PLEKVERDTDRDYYLSAKEALEYGLIDQVVTREEA
Thermus parvatiensis ClpP
(SEQ ID NO: 126)
MVIPYVIEQTARGERVYDIYSRLLKDRIIFLGTPIDAQVA
NVVVAQLLFLDAQNPNQEIKLYINSPGGEVDAGLAIYDTM
QFVRAPVSTIVIGMAASMAAVILAAGEKGRRYALPHAKVM
IHQPWGGVRGTASDIAIQAQEILKAKKLLNEILAKHTGQP
LEKVEKDTDRDYYLSAQEALEYGLIDQVVTREEA
Thermus antranikianii ClpP
(SEQ ID NO: 127)
MVIPYVIEQTARGERVYDIYSRLLKDRIIFLGTPIDSQVA
NVIVAQLLFLDAQNPNQEIRLYINSPGGEVDAGLAIYDTM
QFVRAPVSTIVIGMAASMAAVILAAGEKGRRYALPHSKVM
IHQPWGGVRGTASDIAIQAQEILKAKKLLNEILAKHTGQP
LEKVEKDTDRDYYLSAQEALEYGLIDQVVTREEA
Marinithermus hydrothermalis ClpP
(SEQ ID NO: 128)
MDIFFQLFWLFFIFSALSPYITQQTLFSARARKIAELERK
RGSRVITLIHRQESVSLLGIPLSRFINIDDSEQVLRAIRM
TDKDVPIDLVLHTPGGLVLAAEQIAEALKRHPAKVTVFVP
HYAMSGGTLIALAADEIVMDENAVLGPVDPQLGQYPAAS
ILKVLETKDPKDIEDQTLILADVARKALDQVKRTVKGLLA
DKFGEEKAEEVAALLSQGTWTHDYPISVEEARAMGLPVST
QMPAEVYALMDLYPQAHGGRPSVQYVPIPQQRETPRPTGR
R
Consensus sequence of Thermus ClpP proteins
(SEQ ID NO: 129):
MVIPYVIEQTARGERVYDIYSRLLKDRIIFLGTPIDAQVA
NVIVAQLLFLDAQNPNQEIRLYINSPGGEVDAGLAIYDTM
QFVRAPVSTIVIGMAASMAAVILAAGEKGRRYALPHAKVM
IHQPWGGVRGTASDIAIQAQEILKAKKLLNEILAKHTGQP
LEKVEKDTDRDYYLSAQEALEYGLIDQVVTREEA
Moorella humiferrea ClpP
(SEQ ID NO: 130)
MSILVPVVVEQTNRGERAYDIYSRLLKDRIIFLGSAIDDH
VANLVIAQMLFLEAEDPDKDIHLYINSPGGSISAGMAIFD
TMQYIRPDVSTICVGLAASMGAFLLAAGAKGKRFALPHSE
IMIHQPMGGTQGQAVDIEIHAKRILAIRDTLNRILSDITG
KPVEQIARDTDRDHFMTPLEAKEYGLIDEVITKRELPRK
Moorella thermoacetica ClpP
(SEQ ID NO: 131)
MSVLVPMVVEQTSRGERAYDIYSRLLKDRIIFLGSAIDDH
VANLVIAQMLFLEAEDPDKDIHLYINSPGGSISAGMAIFD
TMQYIRPDVSTICVGLAASMGAFLLAAGAKGKRFALPNSE
IMIHQPMGGTQGQAVDIEIHAKRILAIRDNLNRILSEITG
KPLEQIARDTDRDHFMTAREAREYGLIDEVITKRELPAK
Thermoanaerobacterium thermosaccharolyticum
ClpP
(SEQ ID NO: 132)
MSLVPIVVEQTNRGERSYDIFSRLLKDRIVFLGEEINDVSA
SLVVAQLLFLEGEDPDKDIWLYINSPGGSITSAFAIYDTM
QYIKPDVVTMCVGMAASAGAFLLAAGAKGKRFSLPNSEIM
IHQPLGGTQGQATDIKIHAERIIKMKQKLNKILSERTGQP
LEKIERDTERDFFMDPEEAKAYGLIDDILVRRK
Parageobacillus thermoglucosidasius ClpP
(SEQ ID NO: 133)
MNLIPTVIEQTSRGERAYDIYSRLLKDRIIILGSPIDDQV
ANSIVSQLLFLAAEDPEKDISLYINSPGGSITAGLAIYDT
MQFIKPDVSTICIGMAASMGAFLLAAGAKGKRFALPNSEI
MIHQPLGGAQGQATEIEIAAKRILFLRDKLNRILSENTGQ
PIDVIERDTDRDNFMTAQKAQEYGIIDRVLTRVDEK

The ClpP proteins from certain thermophile and hyperthermophile Archaea may be advantageous, due to the stability requirements for enzymes that are functional at comparatively high temperatures, and/or sequence diversity. Scaffold proteins can be derived from the ClpP of thermophilic and hyperthermophilic Archaea, including, e.g., any of the following proteins:

Pyrococcus furiosus ClpP
(SEQ ID NO: 134)
MDPLSGFVGSLIWWILFFYLLMGPQLQYRQLQIARAKLLE
KMARKRNSTVITMIHRQESIGFFGIPVYKFISIEDSEEVL
RAIRMAPKDKPIDLIIHTPGGLVLAATQIAKALKDHPAET
RVIVPHYAMSGGTLIALAADKIIMDPHAVLGPVDPQLGQ
YPAPSIIKAVEQKGAEKVDDQTLILADVAKKAIKQVQDFL
YDLLKDKYGEEKARELAQILTEGRWTHDYPITVEHARELG
LEVDTNVPEEVYALMELYKQPVRQRGTVEFMPYPVKQEGK
K
Petrotoga halophila ClpP
(SEQ ID NO: 135)
MAIPMPVVIETEGRYERAYDIYSRLLKDRIVFLGTPINDD
VANLIVAQLLFLESQDPDKDIFLYINSPGGSVTAGLGIYD
TMQYVKPDISTICIGQAASMGAVLLAAGTKGKRYSLPYSR
IMIHQPWGGAEGTAMDIQIHAREILRLKDDLNNILSKHTG
QSLEKIEKDTERDFFMNAQEALNYGLIDKVITTKSEATKE
NNKK
Thermococcus chitonophagus ClpP
(SEQ ID NO: 136)
MDPLSGFFGSLIWWFLFLYILLWPQMQYRQLQIMRAKLLQ
KLSRKRNSTVITLIHRQESIGLFGIPVYRFISIEDSEEVL
RAIRMAPKDKPIDLIIHTPGGLVLAATQIAKALKDHPAET
RVIVPHYAMSGGTLIALAADKIIMDPHAVLGPVDPQLGQ
YPAPSILRAVEKKGADKVDDQTLILADVAEKAIRQVRDFI
YNLLKDKYGEEKAKELAQILTEGRGTHDYPITVEEAKKLG
LNVSTDVPEEVYALMELYKQPVRQRGTVEFVPYPVKQESG
KQ
Thermococcus gammatolerans ClpP
(SEQ ID NO: 137)
MDPLSGFLGSLLWWLFFLYILMWPQLQYRQLQIMRAKLLA
KIAKKRNSTVITMIHRQESIGFFGIPVYKFISVEDSEEIL
RAIRAAPKDKPIDLIIHTPGGLVLAATQIARALKEHPAET
RVIVPHYAMSGGTLIALAADRIIMDPNAVLGPVDPQLG
QYPAPSIVKAVEQKGAEKVDDQTLILADVAKKAIKQVQDF
VFYLLKDRYGEEKARQLAQTLTEGRWTHDYPITVDHAKEM
GLHVETDVPEEVYALMELYKQPVRQRGTVEFMPYPVKQEG
AK
Thermococcus kodakarensis ClpP
(SEQ ID NO: 138)
MDPLSGFLGSLLWWLFFLYLLMWPQLQFRALQAARARLMA
QLARKRNSTVIAMIHRQESIGLFGIPVYKFISIEDSEEVL
RAIRSAPKDKPIDLIIHTPGGLVLAATQIARALKEHPAET
RVIVPHYAMSGGTLIALAADKIIMDPNAVLGPVDPQLGQ
YPAPSILRAVEKKGPEKVDDQTLILADVAEKAIKQVQDFV
FSLLKDKYGEEKARELAQILTEGRWTHDYPITVDHARELG
LNVETDVPEEVYALMELYKQPVKQRGTVEFMPYPVKQESK
K
Palaeococcus pacificus ClpP
(SEQ ID NO: 139)
MDPLSGFLGSLIWWLLIFYMLLAPQIQYKQLQLARKKVLE
RLSKKMNSTVITMIHRQESVGLFGIPFYKFISIEDSEEVL
RAIRAAPKDKPINLILHTPGGLVLAATQIAKALKDHPAKT
RVIIPHYAMSGGTLIALAADEIIMDPHAVLGPIDPQLGQ
YPAPSIIKAVERKGADKVDDQTLILADVAEKAIKQVQNFV
YDLLKDKYGEAKAKELAQILTEGRWTHDYPITVEEAKKLG
LNVSTDVPKEVYALMDLYKQPMRQRGTVEFMPYSVNQENK
H
Thermococcus barossii ClpP
(SEQ ID NO: 140)
MNDTTTGLFGSLLWWLFFLYLLLWPQMQYRGLQMARARIL
QRLSKKRGSTVITLIHRQESVGLFGIPFYKFISIEDSEEI
LRAIRMAPKDKPIDLIIHTPGGLVLAATQIARALKDHPAE
TRVIVPHYAMSGGTLIALAADKIIMDPHAVLGPVDPQLG
QYPGPSIVRAVEKKGVDKVDDQTLILADVAEKAIKQVRDL
VYDLLKDRYGEEKARELAQILTEGRWTHDYPITYETAKEL
GLHVETNVPEEVYALMELYKQPMKQRGTVEFMPYTSKGE
NP
Thermococcus piezophilus ClpP
(SEQ ID NO: 141)
MNDTTTGLFGSLLWWLFFLYLLLWPQMQYRGLQMARARIL
QRLSKKRGSTVITLIHRQESVGLFGIPFYKFISIEDSEEI
LRAIRMAPKDKPIDLIIHTPGGLVLAATQIARALKDHPAE
TRVIVPHYAMSGGTLIALAADKIIMDPHAVLGPVDPQLG
QYPGPSIVRAVEKKGVDKVDDQTLILADVAEKAIKQVRDL
VYDLLKDRYGEEKARELAQILTEGRWTHDYPITYETAKEL
GLHVETNVPEEVYALMELYKQPMKQRGTVEFMPYTSKGE
NP
Thermococcus thioreducens ClpP
(SEQ ID NO: 142)
MADATTGFFGSLLWWLFFMYILLWPQMQYRSLQLARAKIL
KRLSEKRGSTVITMIHRQESVGLFGIPFYKFISIEDSEEV
LRAIRAAPKDKPIDLIIHTPGGLVLAATQIAKALHDHPAE
TRVIVPHYAMSGGTLIALAADRIIMDPHAVLGPVDPQL
GQYPGPSIVRAVERKGVDKVDDQTLILADVAEKAIKQVRE
FVYGLLKDRYGEEKARELAQILTEGRWTHDYPITYEHAKE
LGLHVETEVPDEVYALMELYRQPTKQRGTVEFMPYTQKG
ESS
Thermococcus celer ClpP
(SEQ ID NO: 143)
MGDAVSGFFGSLLWWLFLIYLLLWPQMQYRNLQIARIRLL
KRLSEKRKSTVITLIHRQESIGLFGIPFYKFISVEDSEEV
LRAIRSAPKDKPIDLVIHTPGGLVLAATQIAKALHDHPAE
TRVIVPHYAMSGGTLIALAADKIVMDPHAVLGPVDPQL
GQYPGPSIVRAVERKGVDKVDDQTLILADVAEKAIRQVRD
FIYGILKDRYGDEKAKELAQILTEGRWTHDYPITYEHARE
LGLHVSTDVPKEVYALMELYKQPMKQRGTVEFMPYIQRGE
SS
Thermococcus barophilus ClpP
(SEQ ID NO: 144)
MDPLSGFLGSLIWWLFFLYLLLWPQMQYRQLQLMRARLLQ
KLSRKRNSTVITMIHRQESIGLFGIPFYKFISIEDSEEIL
RAIRMAPKDKPIDLIIHTPGGLVLAATQIAKALKDHPAET
RVIIPHYAMSGGTLIALAADKIIMDPHAVLGPVDPQLG
QYPAPSIVRAVEKKGPEKVDDQTLILADVAEKAINQVRNF
VYELLKDKYGEEKAKELAQILTEGRWTHDYPITVEEAQKL
GLHVSTDVPEEVYELMQLYPQPMKQRGTVEFMPYPVRQEK
K
Thermococcus paralvinellae ClpP
(SEQ ID NO: 145)
MDPLSGFLGSLIWWLFFLYLLLWPQMQYRQLQLMRARLLQ
RLSRKRNSTVITMIHRQESIGLFGIPFYKFISIEDSEEIL
RAIRMAPKDKPIDLIIHTPGGLVLAATQIAKALKDHPAET
RVIVPHYAMSGGTLIALAADKIIMDPHAVLGPVDPQLGQ
YPAPSIVRAVQKKGPEKVDDQTLILADVAEKAINQVRNFV
FELLKDKYGEEKAKELAQILTEGRWTHDYPITVEEAKKLG
LHVSTDVPEEVYELMQLYPQPMKQRGTVEFMPYPVKQENK
Thermococcus radiotolerans ClpP
(SEQ ID NO: 146)
MSEAATGFFGSLLWWLFFMYILLWPQMQYRSLQLARAKLL
KRLSEKRKSTVITMIHRQESIGLFGIPFYKFISVEDSEEV
LRAIRSAPKDKPIDLIIHTPGGLVLAATQIAKALHDHPAE
THVIVPHYAMSGGTLIALAADKIIMDPHAVLGPVDPQLG
QYPGPSIVRAVEKKGVDKVDDQTLILADVAEKAIKQVRNF
VYNLLKDRYGEEKAKELAQILTEGRWTHDYPITYEHAKEL
GLHVETDVPEEVYALMELYKQPMKQRGTVEFMPYTQRGES
S

Consensus sequence of Thermococcus ClpP proteins, where “X” is any amino acid that is present at that same position in a

Thermococcus ClpP protein
(SEQ ID NO: 147)
MDPLSGFLGXLLWWWLFXYXLLXXXMQYXQLQXMRRKLLX
KLXRKRNSTVIXMIHXQESIGXFGIPXYXFXSIEDXEEVL
RAIRXAPXDKPXDLIIHXPGGLVLAATQIAKALKDHPXET
RVIIPHYXMSGGTLIALAADDIIMDPHXVLGPXDPXLGQY
PXPXIIRAVEEKGXEKVDDQTLILADXAEKAIXQVQNFIY
YLLKDKYGEEKAKELAQXLTEGRXXHXYPXTVXEAKKLGL
HXXTDVPXEVYXLMXLYXQPXRQRGTVEFXPYXVKQEE
Pyrodictium delaneyi ClpP
(SEQ ID NO: 148)
MIFFLFWLLLLFSIMEPILSLRRLQAARLALIRQMEQKYG
WRVVTLIHREERVTFFGIPIQRFIDIDDSEAVLRAIRTTP
PDKPIALILHTPGGLVLAASQIARALKRHPGRKIVIVPHY
AMSGGTLIALAADEILMDPNAVLGPLDPQLSLGPQGPVV
PAPSILKVAKMKGDKASDTTLIVADIAEKAIMEMQEVITD
LLKDKMGEEKAREIAKVLTEGKWTHDYPITVEKAKELGLP
VKTEVPPEVYQLMELYPQAPHNRPGVEFIPQPLPQHPVRR
GQRATS
Pyrodictium occultum ClpP
(SEQ ID NO: 149)
MKGDAAGSIISLLFWLLLLIALMEPALSVRRLQAARLSLI
KNMERKYGWRVVTMIHREERVTFFGIPLQRFIDIDDSEAV
LRAIRTTPPDKPIALILHTPGGLVLAASQIAMALKRHPGR
KIVIIPHYAMSGGTLIALAADEILMDPNAVLGPLDPQLSL
GPQGPVVPAPSVLRAAEVKGDKASDTTLIIADIARKAIAE
MQETIVELLRDKMGEERAREIAKTLTEGRWTHDYPITPEK
ARELGLPVKTEVPPEVYELMELYPQAPGNRPGVEFIPQPL
PHQPPHRGHSGK

Podospora anserina ClpP
(SEQ ID NO: 150)
MNTQRTAFHLLRRLGASHCRRTSKFSTFPGGIPPTSGGIP
MPYITEVTAGGWRTSDIFSKLLQERIVCLNGAIDDTVSAS
IVAQLLWLESDNPDKPITMYINSPGGEVSSGLAIYDTMTY
IKSPVSTVCVGGAASMAAILLIGGEPGKRYALPHSSIMV
HQPLGGTRGQASDILIYANQIQRLRDQINKIVQSHINKSF
GFEKYDMQAINDMMERDKYLTAEEAKDFGIIDEILHRRVK
NDGTMLSADAKEGKH
Colletotrichum orbiculare ClpP
(SEQ ID NO: 151)
MNCQRTLFRALRAAPAASLRRHARAFTNFPAGLPGGAPPV
GSIPLPYITEVSSSGWRTYDIFSKLLQERIVCLNGAIDDT
VSASIVAQLLWLESDSPDKAITMYINSPGGSVSSGLAIYD
TMTYIKSPVSTVCLGAASSMAALLLTGGEAGKRYALPHSS
VMIHQPLGGTQGQASDILIYANQIQRIRKQINEIMKRHIN
KSFGHEKFNLEEVNDMMERDKYLTAEEAKEIGVIDEILTR
REEKDAKEKDSAEEQKTKP
Purpureocillium lilacinum ClpP
(SEQ ID NO: 152)
MALRQRVLPALRMLPCRQVRAFGFSSAPGNTAPTQDYIPM
PYIEETSAAGRKTWDIFSKLLQERIVCLNGEINDYMSASI
VAQLLWLESDTPEKPITMYINSPGGSVTSGMAIYDTMTYI
KSPVSTVCVGGAASMAAILLAGGEAGQRYALPHSSIMIHQ
PLGGTRGQASDILIYANQIQRIREQSNKIMQHHLNKAKGY
DKYSIDEVNDMMERDKYLSVAEALDLGVIDEILTKRADKD
PKKEEASASPAGQDSR
Lomentospora prolificans ClpP
(SEQ ID NO: 153)
MSFQRTLSRAVRGATRRPARSASALRLPTATRQYHASAPP
SGIIPIPYITEVTSGGWRTSDIFSKLLQERIVCLYGSIDD
GTAASIVSQLLWLEAENPDKPITLYINSPGGMISSGLAIY
DTMSYIRPPVSTVCVGAASSMAALLLVGGEAGQRFALPHS
SIMIHQPLGGTQGQASDILIYANQIQRIRDQVNEIYRYHV
NKALGSDKFDQKSVSDLMERDKYLTPEEAKELGIIDEILS
KRPVPVEGQEGSDVK

In certain embodiments, ClpP proteins from fungi that are thermophiles may be advantageous, due to the stability requirements for enzymes that are functional at comparatively high temperatures. Scaffold proteins can be derived from the ClpP of thermophilic fungi, including, e.g., Thermothelomyces thermophilus ClpP having the sequence shown below.

Thermothelomyces thermophilus ClpP
(SEQ ID NO: 154)
MNTQRSAFRLLKRIGDTARCRNFSKFSASSRPIPPLGNIP
MPYITEVTSGGWRTSDIFSKLLQERIVCLNGAIDDTVSAS
IVAQLLWLESDNPDKPITMYINSPGGEVSSGLAIYDTMTY
IKSPVSTVCVGGAASMAAILLIGGEPGKRYALQHSSIMV
HQPLGGTRGQAADILIYANQIQRIREQINKIVQTHVNRAF
GYEKFDMKAINDMMERDRYLTADEAKEMGIIDEILHKREK
GEDKPGVGDGKVKL.

### VI. Polynucleotides and Expression Constructs

The engineered SARS-CoV-2 RBD polypeptides, related vaccine fusion compositions, and other scaffolded proteins described herein are typically produced by first generating expression constructs (i.e., expression vectors) that contain operably linked coding sequences of the various structural components described herein. Alternatively, nucleic acid molecules encoding and expressing the immunogen polypeptides and the fusion proteins can be used directly in vaccine compositions, e.g., in mRNA nanoparticles or DNA vaccines. Accordingly, in some related aspects, the invention provides substantially purified polynucleotides (DNA or RNA) that encode the immunogens or nanoparticle displayed immunogens as described herein. Some polynucleotides of the invention encode one of the engineered RBD immunogen polypeptides described herein, e.g., SEQ ID NO:3. Some polynucleotides of the invention encode the subunit sequence of one of the nanoparticle scaffolded vaccines described herein, e.g., the fusion protein sequences shown in SEQ ID NOs:11-16. While the expressed RBD immunogen polypeptides of the invention typically do not contain the N-terminal leader sequence, some of the polynucleotide sequences of the invention additionally encode the leader sequence of the native spike protein. Thus, for example, polynucleotides encoding engineered SARS-CoV-2 RBD immunogen polypeptides (e.g., SEQ ID NO:3) or the scaffolded polypeptide sequences (e.g., SEQ ID NOs:11-22) can additionally encode a leader sequence such as the Ig leader sequence shown in SEQ ID NO:27 (MKHLWFFLLLVAAPRWVLS), or a substantially identical or conservatively modified variant sequence.

Also provided in the invention are expression vectors that harbor such polynucleotides (e.g., CMV vectors exemplified herein) and host cells for producing the vaccine immunogens (e.g., HEK293F, ExpiCHO, and CHO-S cell lines exemplified herein). The fusion polypeptides encoded by the polynucleotides or expressed from the vectors are also included in the invention. As described herein, the nanoparticle subunit fused soluble S immunogen polypeptides will self-assemble into nanoparticle vaccines that display the immunogen polypeptides or proteins on its surface.

The polynucleotides and related vectors can be readily generated with standard molecular biology techniques or the protocols exemplified herein. For example, general protocols for cloning, transfecting, transient gene expression and obtaining stable transfected cell lines are described in the art, e.g., Sambrook et al., Molecular Cloning: A Laboratory Manual, Cold Spring Harbor Press, N.Y., (3^{rd} ed., 2000); and Brent et al., Current Protocols in Molecular Biology, John Wiley & Sons, Inc. (ringbou ed., 2003). Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, C A, 1990; Mattila et al., Nucleic Acids Res. 19:967, 1991; and Eckert et al., PCR Methods and Applications 1:17, 1991.

The selection of a particular vector depends upon the intended use of the fusion polypeptides. For example, the selected vector must be capable of driving expression of the fusion polypeptide in the desired cell type, whether that cell type be prokaryotic or eukaryotic. Many vectors contain sequences allowing both prokaryotic vector replication and eukaryotic expression of operably linked gene sequences. Vectors useful for the invention may be autonomously replicating, that is, the vector exists extrachromosomally and its replication is not necessarily directly linked to the replication of the host cell's genome. Alternatively, the replication of the vector may be linked to the replication of the host's chromosomal DNA, for example, the vector may be integrated into the chromosome of the host cell as achieved by retroviral vectors and in stably transfected cell lines. Both viral-based and nonviral expression vectors can be used to produce the immunogens in a mammalian host cell. Nonviral vectors and systems include plasmids, episomal vectors, typically with an expression cassette for expressing a protein or RNA, and human artificial chromosomes (see, e.g., Harrington et al., Nat. Genet. 15:345, 1997). Useful viral vectors include vectors based on lentiviruses or other retroviruses, adenoviruses, adeno-associated viruses, Cytomegalovirus, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

Depending on the specific vector used for expressing the fusion polypeptide, various known cells or cell lines can be employed in the practice of the invention. The host cell can be any cell into which recombinant vectors carrying a fusion of the invention may be introduced and wherein the vectors are permitted to drive the expression of the fusion polypeptide is useful for the invention. It may be prokaryotic, such as any of a number of bacterial strains, or may be eukaryotic, such as yeast or other fungal cells, insect or amphibian cells, or mammalian cells including, for example, rodent, simian or human cells. In some embodiments, the employed host cell is derived from yeast. This include cells from, e.g., Kluyveromyces lactis, Pichia pastoris, Yarrowia lipolytica and Saccharomyces cerevisiae. In some other embodiments, the employed host cell is a mammalian cell. In various embodiments, cells expressing the fusion polypeptides of the invention may be primary cultured cells or may be an established cell line. Thus, in addition to the cell lines exemplified herein (e.g., CHO cells), a number of other host cell lines well known in the art may also be used in the practice of the invention. These include, e.g., various Cos cell lines, HeLa cells, Sf9 cells, HEK293, AtT20, BV2, and N18 cells, myeloma cell lines, transformed B-cells and hybridomas.

The use of mammalian tissue cell culture to express polypeptides is discussed generally in, e.g., Winnacker, From Genes to Clones, VCH Publishers, N.Y., N.Y., 1987. The fusion polypeptide-expressing vectors may be introduced to the selected host cells by any of a number of suitable methods known to those skilled in the art. For the introduction of fusion polypeptide-encoding vectors to mammalian cells, the method used will depend upon the form of the vector. For plasmid vectors, DNA encoding the fusion polypeptide sequences may be introduced by any of a number of transfection methods, including, for example, lipid-mediated transfection (“lipofection”), DEAE-dextran-mediated transfection, electroporation or calcium phosphate precipitation. These methods are detailed, for example, in Brent et al., supra. Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, Life Technologies, JBL Scientific, MBI Fermentas, PanVera, Promega, Quantum Biotechnologies, Sigma-Aldrich, and Wako Chemicals USA.

For long-term, high-yield production of recombinant fusion polypeptides, stable expression is preferred. Rather than using expression vectors which contain viral origins of replication, host cells can be transformed with the fusion polypeptide-encoding sequences controlled by appropriate expression control elements (e.g., promoter, enhancer, sequences, transcription terminators, polyadenylation sites, etc.), and selectable markers. The selectable marker in the recombinant vector confers resistance to the selection and allows cells to stably integrate the vector into their chromosomes. Commonly used selectable markers include neo, which confers resistance to the aminoglycoside G-418 (Colberre-Garapin, et al., J. Mol. Biol., 150:1, 1981); and hygro, which confers resistance to hygromycin (Santerre et al., Gene, 30: 147, 1984). Through appropriate selections, the transfected cells can contain integrated copies of the fusion polypeptide encoding sequence.

### VII. Pharmaceutical Compositions and Therapeutic Applications

In another aspect, the invention provides pharmaceutical compositions and related therapeutic methods of using the engineered coronavirus S immunogens and nanoparticle vaccine compositions as described herein. In various embodiments, the pharmaceutical compositions can contain the engineered RBD polypeptides, nanoparticle scaffolded viral RBD immunogens, as well as polynucleotide sequences or vectors encoding the engineered viral RBD immunogens or nanoparticle vaccines described herein. In some embodiments, the engineered RBD immunogens can be used for preventing and treating the SARS-CoV-2 infections. In various other embodiments, the nanoparticle vaccines containing different viral or non-viral immunogens described herein can be employed to prevent or treat the corresponding diseases, e.g., infections caused by the various coronaviruses. Some embodiments of the invention relate to use of the engineered SARS-CoV-2 RBD immunogens or vaccines for preventing or treating SARS-CoV-2 infections in human subjects.

In some embodiments, the engineered RBD immunogens and related fusion proteins can be used for detection of antibodies against SARS-CoV-2. These immunogens or fusion proteins can be provided in kits. The kits can additionally include other components, reagents and/or instructions that are needed or useful for detecting antibodies against SARS-CoV-2. In some other embodiments, the invention provides related methods for detecting antibodies against SARS-CoV-2. Some of these methods entail detection of binding of an SARS-CoV-2 antibody to an engineered RBD immunogen (or a related fusion protein) that is immobilized to a solid surface. Some of these methods entail detection of binding of an engineered RBD immunogen (or a related fusion protein) to an immobilized antibody-containing sample obtained from a human subject. Some of these methods entail detection of the ability of a sample containing antibodies from a human subject to block the binding of an engineered RBD immunogen (or a related fusion protein) to an immobilized ACE2 protein (or a modified variant). Some of these methods entail detection of the ability of a sample containing antibodies from a human subject to block the binding of ACE2 protein (or a modified variant) to an engineered RBD immunogen (or a related fusion protein) that is immobilized to a solid surface.

In the practice of the various therapeutic methods of the invention, the subjects in need of prevention or treatment of a disease or condition (e.g., SARS-CoV-2 infection) is administered with the corresponding nanoparticle vaccine, the immunogen protein or polypeptide, or an encoding polynucleotide described herein. Typically, the scaffolded vaccine, the immunogen protein or the encoding polynucleotide disclosed herein is included in a pharmaceutical composition. The pharmaceutical composition can be either a therapeutic formulation or a prophylactic formulation. Typically, the composition can additionally include one or more pharmaceutically acceptable vehicles and, optionally, other therapeutic ingredients (for example, antiviral drugs). Various pharmaceutically acceptable additives can also be used in the compositions.

Thus, some of the pharmaceutical compositions of the invention are vaccine compositions. For vaccine compositions, appropriate adjuvants can be additionally included. Examples of suitable adjuvants include, e.g., aluminum hydroxide, lecithin, Freund's adjuvant, MPL™ and IL-12. In some embodiments, the vaccine compositions or nanoparticle immunogens disclosed herein can be formulated as a controlled-release or time-release formulation. This can be achieved in a composition that contains a slow release polymer or via a microencapsulated delivery system or bioadhesive gel. The various pharmaceutical compositions can be prepared in accordance with standard procedures well known in the art. See, e.g., Remington's Pharmaceutical Sciences, 19^{th} Ed., Mack Publishing Company, Easton, Pa., 1995; Sustained and Controlled Release Drug Delivery Systems, J. R. Robinson, ed., Marcel Dekker, Inc., New York, 1978); U.S. Pat. Nos. 4,652,441 and 4,917,893; 4,677,191 and 4,728,721; and 4,675,189.

The pharmaceutical compositions of the invention can be readily employed in a variety of therapeutic or prophylactic applications, e.g., for treating SARS-CoV-2 infection or eliciting an immune response against SARS-CoV-2 in a subject. In various embodiments, the vaccine compositions can be used for treating or preventing infections caused by a pathogen from which the displayed immunogen polypeptide in the nanoparticle vaccine is derived. Thus, the vaccine compositions of the invention can be used in diverse clinical settings for treating or preventing infections caused by various viruses. As exemplification, a SARS-CoV-2 nanoparticle vaccine composition can be administered to a subject to induce an immune response to SARS-CoV-2, e.g., to induce production of neutralizing antibodies to the virus. For subjects at risk of developing an SARS-CoV-2 infection, a vaccine composition of the invention can be administered to provide prophylactic protection against viral infection. Therapeutic and prophylactic applications of vaccines derived from the other immunogens described herein can be similarly performed. Depending on the specific subject and conditions, pharmaceutical compositions of the invention can be administered to subjects by a variety of administration modes known to the person of ordinary skill in the art, for example, intramuscular, subcutaneous, intravenous, intra-arterial, intra-articular, intraperitoneal, or parenteral routes.

In general, the pharmaceutical composition is administered to a subject in need of such treatment for a time and under conditions sufficient to prevent, inhibit, and/or ameliorate a selected disease or condition or one or more symptom(s) thereof. In various embodiments, the therapeutic methods of the invention relate to methods of blocking the entry of SARS-CoV-2 into a host cell, e.g., a human host cell, methods of preventing the S protein of a coronavirus from binding the host receptor, and methods of treating acute respiratory distress that is often associated with coronavirus infections. In some embodiments, the therapeutic methods and compositions described herein can be employed in combination with other known therapeutic agents and/or modalities useful for treating or preventing coronavirus infections. The known therapeutic agents and/or modalities include, e.g., a nuclease analog or a protease inhibitor (e.g., remdesivir), monoclonal antibodies directed against one or more coronaviruses, an immunosuppressant or anti-inflammatory drug (e.g., sarilumab or tocilizumab), ACE inhibitors, vasodilators, or any combination thereof.

For therapeutic applications, the compositions should contain a therapeutically effective amount of the nanoparticle scaffolded immunogen described herein. For prophylactic applications, the compositions should contain a prophylactically effective amount of the nanoparticle immunogen described herein. The appropriate amount of the immunogen can be determined based on the specific disease or condition to be treated or prevented, severity, age of the subject, and other personal attributes of the specific subject (e.g., the general state of the subject's health and the robustness of the subject's immune system). Determination of effective dosages is additionally guided with animal model studies followed up by human clinical trials and is guided by administration protocols that significantly reduce the occurrence or severity of targeted disease symptoms or conditions in the subject.

For prophylactic applications, the immunogenic composition is provided in advance of any symptom, for example in advance of infection. The prophylactic administration of the immunogenic compositions serves to prevent or ameliorate any subsequent infection. Thus, in some embodiments, a subject to be treated is one who has, or is at risk for developing, an SARS-CoV-2 infection, for example because of exposure or the possibility of exposure to the SARS-CoV-2 virus. Following administration of a therapeutically effective amount of the disclosed therapeutic compositions, the subject can be monitored for SARS-CoV-2 infection, symptoms associated with SARS-CoV-2 infection, or both.

For therapeutic applications, the immunogenic composition is provided at or after the onset of a symptom of disease or infection, for example after development of a symptom of SARS-CoV-2 infection, or after diagnosis of the infection. The immunogenic composition can thus be provided prior to the anticipated exposure to the virus so as to attenuate the anticipated severity, duration or extent of an infection and/or associated disease symptoms, after exposure or suspected exposure to the virus, or after the actual initiation of an infection. The pharmaceutical composition of the invention can be combined with other agents known in the art for treating or preventing infections by a SARS-CoV-2.

The nanoparticle vaccine compositions containing novel structural components as described in the invention or pharmaceutical compositions of the invention can be provided as components of a kit. Optionally, such a kit includes additional components including packaging, instructions and various other reagents, such as buffers, substrates, antibodies or ligands, such as control antibodies or ligands, and detection reagents. An optional instruction sheet can be additionally provided in the kits.

### EXAMPLES

The following examples are offered to illustrate, but not to limit the present invention.

### Example 1—SARS-CoV-2 RBD Elicits Neutralizing Antibodies

FIG. 2 demonstrates that an unmodified RBD, multimerized by conjugating to keyhole limpet hemocynanin, elicits robust responses in rats. Specifically, rats immunized in two rounds elicited neutralizing responses equivalent to greater than 100 ug/ml ACE2-Ig, a point inhibitor of infection. Critically, FIG. 2 shows that the RBD elicits a more potent neutralizing response than the soluble S-protein ectodomain, when conjugated to one of two scaffolds, namely KLH (as in FIG. 2) or the mi3 60-mer scaffold. Note first that the 60-mer scaffold elicits a more potent response than KLH, and that that in all cases wild-type RBD is used, and that all multimers are chemically conjugated (i.e. not fusion proteins).

### Example 2—Improved Expression of Engineered RBD Proteins

It was observed that expression of the wild-type RBD as a fusion protein (as distinct from a chemical conjugate) poses difficulties because most multimeric constructs where the antigen is the wild-type SARS-CoV-2 RBD aggregate in the cell and do not express.

We also compared the wild-type RBD to a modified variant, the sequence of which is described below (SEQ ID NO:3), that we call “gRBD.” Relative to the wildtype sequence, SEQ ID NO:3 contains four engineered glycosylation sites at residues 370, 394, 428, and 517.

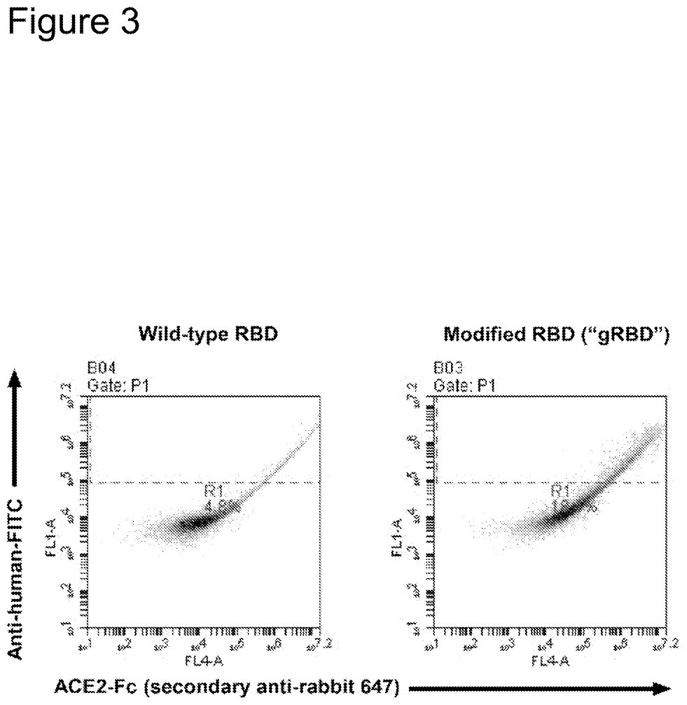

For instance, we expressed the RBD as a fusion protein with an Fc domain with a transmembrane region derived from PDGFR, and measured cell surface expression by flow cytometry (FIG. 3). In the context of a fusion protein with an Fc dimerization domain and a transmembrane region, the modified gRBD (SEQ ID NO:3) containing four engineered glycosylation sites at residues 370, 394, 428, and 517 expressed approximately 4-fold more efficiently than an otherwise identical transmembrane construct based on the wild-type RBD. Thus, the gRBD greatly enhances expression, e.g., in contexts that include a dimerization domain and/or a transmembrane domain.

Notably, the transmembrane region derived from PGDRF is but one such means of anchoring the gRBD to the surface of a cell. Other transmembrane regions are known in the art, and may be derived from, e.g., cytomegalovirus glycoprotein B (gB), influenza HA, influenza neuraminidase, measles H, measles F, vesicular stomatitis virus G, and coronavirus S proteins including that of SARS-CoV-2. Indeed, viral transmembrane regions may comprise epitopes capable of being recognized by CD4+ T cells. In addition to transmembrane regions, a glycosylphosphatidylinositol (GPI) anchor may be used to anchor the gRBD to the surface of a cell. Generating a fusion protein containing the gRBD antigen and a GPI signal sequence provides a means of anchoring the gRBD antigen to the surface of a cell.

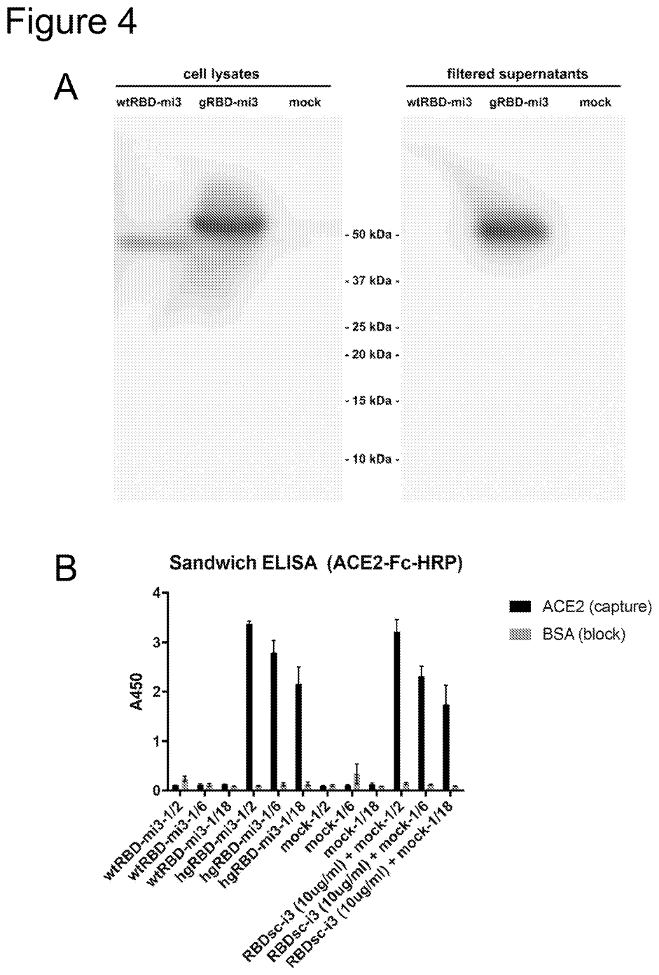

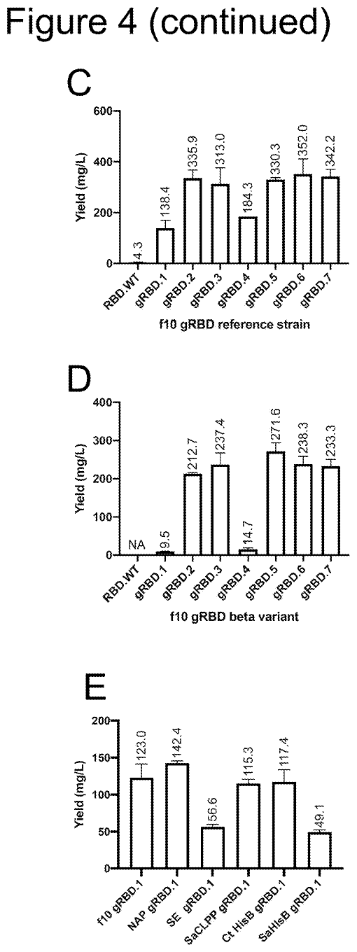

The improved expression of the gRBD relative to the wild-type RBD was especially profound in the context of a 60-mer self-assembling multimerization scaffold. The wild-type SARS-CoV-2 RBD or the gRBD were fused to the N-terminus of the mi3 60-mer self-assembling multimer. The wild-type RBD-mi3 60-mer fusion expressed at quite paltry levels in comparison to the gRBD-mi3 60-mer (FIG. 4A-B). Indeed, the wild-type RBD material was no longer detectable after filtration, suggesting that all or nearly all of the material observed without filtration was aggregated (FIG. 4A). Similar observations were made using an sc-i3 scaffold as for using the mi3 scaffold (FIG. 4B).

Similar observations also were made for fusion proteins containing RBDs and the F10 scaffold. The wild-type RBD of the reference sequence or gRBD versions derived from the reference sequence containing different amino acid substitutions (gRBD.1, gRBD.2, gRBD.3, gRBD.4, gRBD.5, gRBD.6, and gRBD.7) were cloned onto the C-terminus of the F10 scaffold, and expressed by transfection of HEK293T cells, and the concentrations of F10-gRBD versions was determined in supernatants by ELISA (FIG. 4C). The F10-gRBD versions derived from the reference strain all expressed at substantially higher concentrations than the RBD with the wild-type sequence of the reference strain. Next, F10-gRBD versions were generated that were based on the sequence of the beta variant of SARS-CoV-2. Again, the F10-gRBD versions were expressed by transfection of HEK293T cells, and the concentrations of F10-gRBD versions was determined in supernatants by ELISA (FIG. 4D). For the beta variant, the concentrations of each version detected in supernatants were undetectable for the wild-type RBD, 9.5 mg/L for gRBD.1, 212.7 mg/L for gRBD.2, 237.4 mg/L for gRBD.3, 14.7 mg/L for gRBD.4, 217.6 mg/L for gRBD.5, 283.3 mg/L for gRBD.6, 233.3 mg/L for gRBD.7. Thus, gRBD versions gRBD.2, gRBD.3, gRBD.5, gRBD.6, and gRBD.7 may generally tolerate variation in the sequence of the gRBD, e.g., due to the inclusion of substitutions from different variants of SARS-CoV-2.

Fusion proteins were generated based on gRBD.1 and various self-assembling scaffold proteins and compared for expression efficiency. The gRBD.1 and self-assembling scaffold protein fusions compared were F10-gRBD.1, NAP-gRBD.1, Salmonella enterica (SE) Dps (SE-gRBD.1), Staphylococcus aureus (SA) ClpP (SEQ ID NO:97) (SaClpP-gRBD.1), the HisB of the thermophilic fungi Chaetomium thermophilum (SEQ ID NO:95) (Ct HisB-gRBD.1), and Staphylococcus aureus HisB (SEQ ID NO:34) (SaHisB-gRBD.1). Among these, the concentrations detected in supernatants were 123.0 mg/L for F10-gRBD.1, 142.4 mg/L for NAP-gRBD.1, 56.6 mg/L for SE-gRBD.1, 115.3 for SaClpP-gRBD.1, 117.4 mg/L for CtHisB-gRBD.1, and 49.1 for SaHisB-gRBD.1 (FIG. 4E). Thus, gRBD can be expressed on multiple self-assembling scaffold platforms.

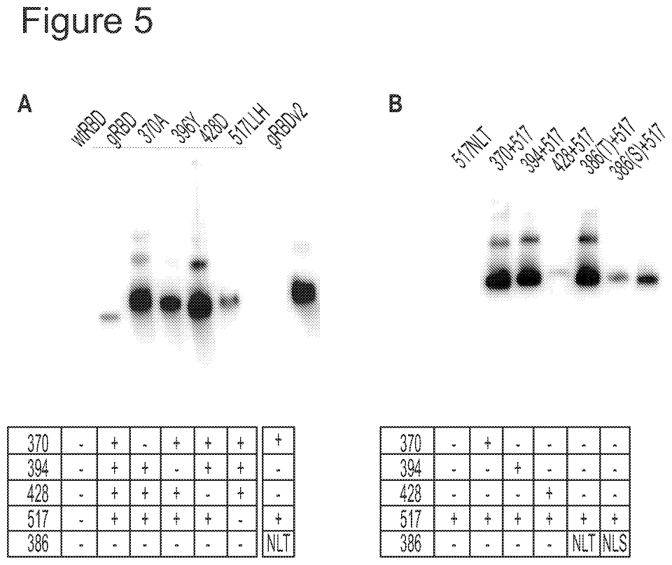

SARS CoV-2 RBD proteins were fused to the C-terminus of the NAP scaffold protein and expressed in Expi293 cells. NAP (neutrophil-activating protein) is a Dps protein from Helicobacter pylori. The NAP scaffold expresses as a self-assembling 12-mer. The yield and fidelity of particle production by NAP-RBD fusion proteins based on different RBD variants was assessed by native protein gel Western blot (FIG. 5). The NAP-RBD variants included the wild-type RBD, gRBD (with engineered glycosylation sites at residues 370, 394, 428, and 517), and variants in which the glycans at these sites were individually reverted, were assessed for particle production yield fidelity (FIG. 5A). Whereas the NAP-RBD with wild-type RBD sequences expressed at low levels, high expression was seen for the variants with 3-4 N-linked glycans. This experiment suggested that the four engineered glycosylation sites maximized expression. Pairwise combinations of engineered glycosylation sites that include the engineered glycan at position 517 also were evaluated (FIG. 5B). The engineered glycan at position 517 alone greatly improved NAP-RBD expression. Thus, a glycan at position 517, introduced through the combinations of substitutions that engineer a glycan at position 517 (L517N/H519T or L517N/H519S), greatly enhance RBD expression, particularly in the context of a self-assembling homo-multimer such as NAP.

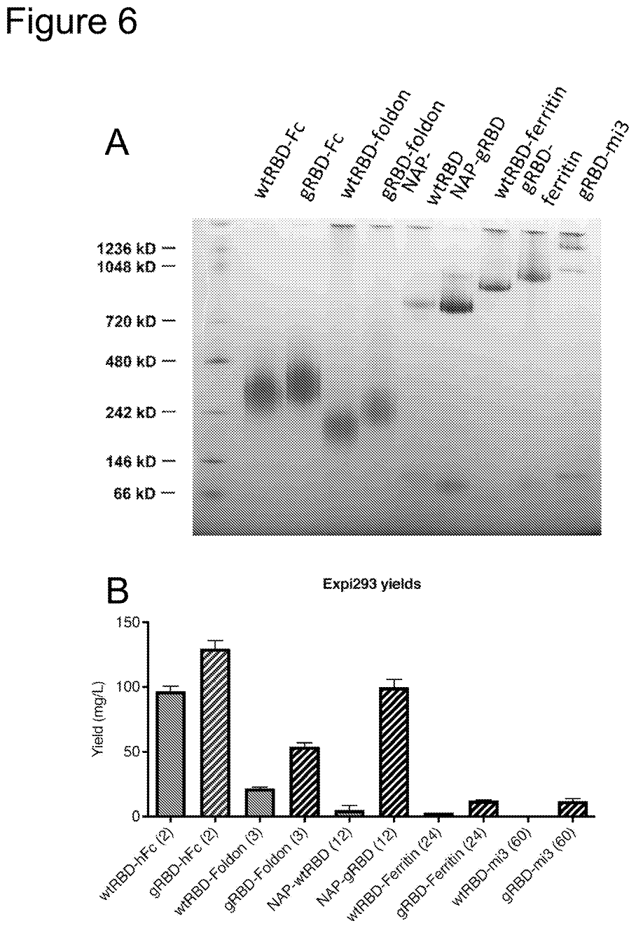

The gRBD antigen with four engineered glycosylation sites was expressed in the context of five different dimerization, trimerization, and multimerization domains. These included gRBD-Fc (dimer), gRBD-foldon (trimer), NAP-gRBD (12-mer, ferritin (24-mer), and mi3 (60-mer) (Table 1). Native protein gel electrophoresis demonstrated particle assembly for the various gRBD fusion proteins (FIG. 6A). Yields were substantially improved for the gRBD relative to the wild-type RBD protein fused to every dimerization, trimerization, and multimerization platform (FIG. 6B). Indeed, at the 60-mer level (mi3), detectable expression of RBD-mi3 was not observed. By contrast, gRBD-mi3 expressed. Thus, the engineered glycosylation sites present in the gRBD enable expression in the context of multimerization scaffolds.

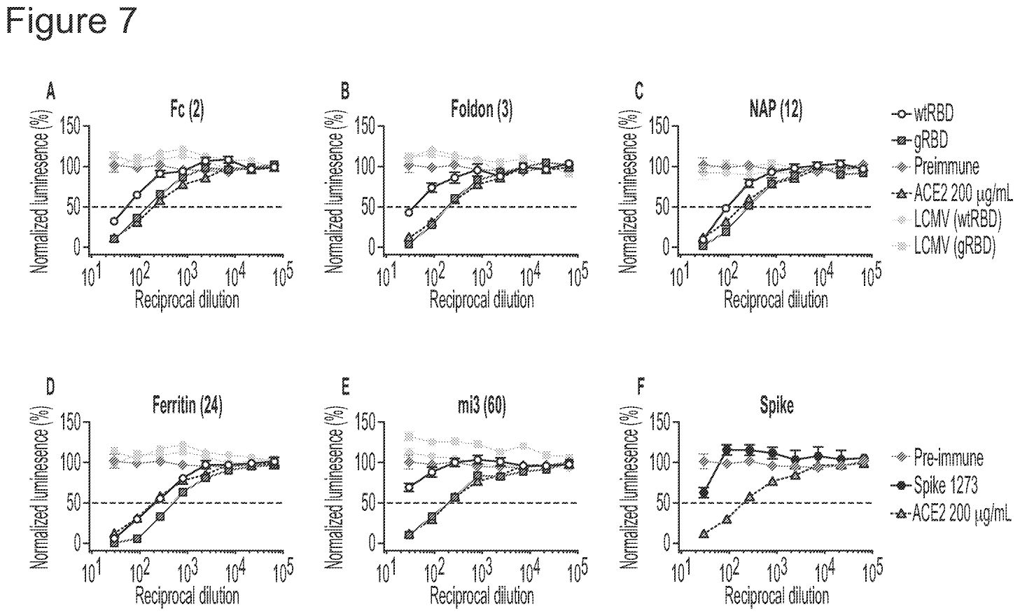

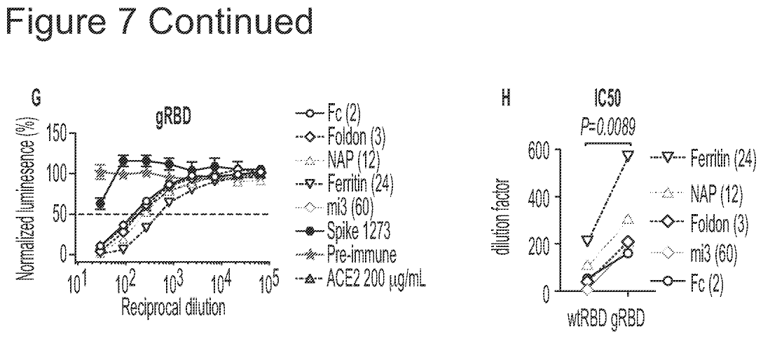

The gRBD-scaffold fusion proteins were evaluated for their potential to elicit neutralizing antibody responses after vaccination in mice. Five mice per group were electroporated with 60 μg/hind leg of plasmid DNA expressing wtRBD or gRBD on days 0 and 14. Serum was evaluated for neutralization of SARS-CoV-2 pseudoviruses on day 21. Neutralization was evaluated for animals immunized with wtRBD or gRBD fused to the Fc dimer (FIG. 7A), foldon trimer (FIG. 7B), H. pylori NAP 12-mer (FIG. 7C), H. pylori ferritin 24-mer (FIG. 7D), and mi3 60-mer (FIG. 7E). An additional control group was electroporated with a plasmid expressing SARS-CoV-2 spike (S) protein containing two stabilizing prolines (FIG. 7F). Pooled preimmune sera, and pooled preimmune sera doped with 200 μg/mL of ACE2-Fc were used as negative and positive controls. (FIG. 7G) Neutralizing potency varied by platform with higher-order multimerization generally favored, perhaps until reaching the 60-mer level, as the neutralization titers were approximately 60-mer=dimer<trimer<12-mer<24-mer. (FIG. 7H). Importantly, the gRBD elicited more potent neutralizing antibody responses than the wtRBD for every scaffold platform.

As shown in FIG. 3, this modified variant expresses much more efficiently on the surface of transfected HEK293T cells than the wild-type RBD sequence. A model of gRBD and its sequence are provided in FIG. 1. The key strength of gRBD is shown in FIGS. 4-6, namely when is expressed as a fusion construct with a multimerizing carrier such as mi3 (60-mer) or ferritin (24-mer), the resulting construct expresses much more efficiently than the wild-type RBD. Moreover, modified gRBD antigens elicited much more potent neutralizing antibody responses after vaccination of animals than unmodified RBD or minimally-modified S protein (FIG. 7). This suggests that this RBD variant, gRBD, and related variants or derivatives, will provide much better vaccines when expressed with a viral vector or with mRNA nanoparticles than the wild-type RBD, and that the same construct can be much more efficiently expressed as a recombinant protein vaccine when expressed in eukaryotic cells (for example yeast, CHO, or 239T cells).

### Example 3—Antigenic Properties of the Engineered gRBD

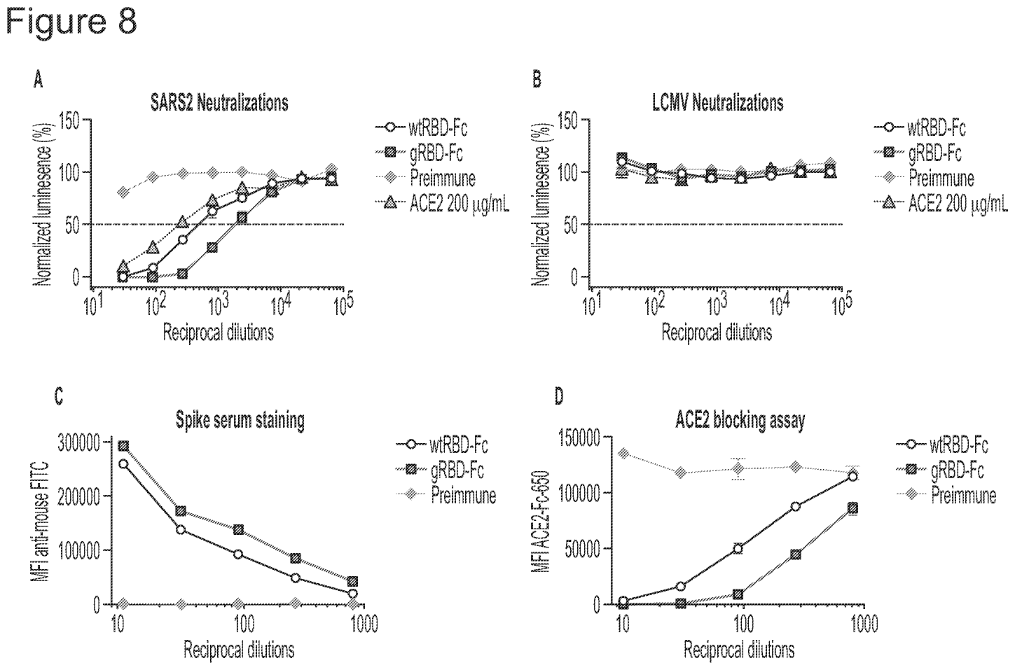

In addition to assembling more efficiently, the gRBD elicits neutralizing antibody responses more effectively than the wild-type RBD. In order to express a purified form of the wild-type RBD that was not a monomer and could be compared directly against the gRBD, the wild-type RBD and gRBD were expressed as Fc fusion proteins. The wild-type RBD and gRBD Fc fusion proteins were purified first by protein A purification, and then by size-exclusion chromatography (SEC). 25 μg of each protein was combined with 25 μg of the adjuvant MPLA and 10 μg of the adjuvant QS-21, and administered to mice by intramuscular injection. Despite having controlled for the total amount of protein expressed, and eliminated aggregated protein by SEC, the gRBD-Fc elicited antibodies that neutralized SARS-CoV-2 pseudoviruses at higher titers than the wild-type RBD-Fc antigen (FIG. 8A). No neutralization was observed against an LCMV pseudovirus negative control (FIG. 8B). The antibodies elicited by immunization with gRBD-Fc bound to cells expressing SARS-CoV-2 spike (S) protein more efficiently than those elicited by immunization with the wild-type RBD-Fc (FIG. 8C). In addition, the antibodies elicited by the gRBD-Fc were more effective than those elicited by the wild-type RBD-Fc at blocking the ability of the SARS-CoV-2 S protein to bind its receptor ACE2 (FIG. 8D). Therefore, in addition to the improved expression of gRBD versus wild-type RBD protein antigens, the gRBD is more effective at eliciting neutralizing antibodies than the wild-type RBD. Without the intention of being limited by any particular theory, the gRBD may be more effective at eliciting neutralizing antibody responses than the wild-type RBD, even after controlling for the amount of protein present and removing aggregates, due to improving the stability of the native conformation of the RBD, hindering antibody access to undesired epitopes, and/or interactions between the engineered glycans and receptors expressed on antigen-presenting cells (APCs).

### Example 4—Fusions of the 2RBD onto the C-Termini of Self-Assembling Multimer Scaffolds

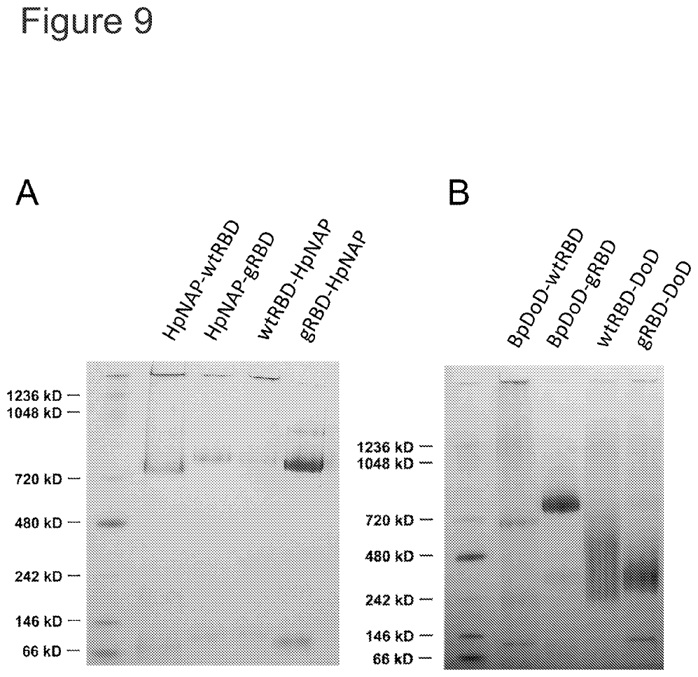

Fusion of the gRBD antigen to the C-terminus rather than the N-terminus of a self-assembling multimer scaffold greatly improved expression and the fidelity of multimerization. The wild-type RBD and the gRBD were fused to the N- and C-termini of two different self-assembling homo-multimer scaffolds that each have both the N- and C-termini available for fusion (FIG. 9). Fusing the gRBD to the C-terminus of NAP, as self-assembling 12-mer from Helicobacter pylori, greatly increased expression and multimerization fidelity (FIG. 9A). Notably, the wild-type RBD was sufficiently prone to aggregation that fusion of the wild-type RBD to the C-terminus of NAP did not appear to substantially improve expression or multimer assembly. Similar observations were made when the wild-type RBD and gRBD were fused to the N- and C-termini of the 12-mer dodecin from Bordetella pertussis (BpDoD) (FIG. 9B). Fusing the gRBD to the C-terminus of BpDoD greatly improved its expression and the fidelity of homo-multimer self-assembly. However, the fidelity of homo-multimer self-assembly was far from optimal for both N- and C-terminal fusions of the wild-type RBD. Thus, fusions to the N- and C-termini of the same self-assembling homo-multimer scaffold reveal two observations: First, the gRBD is capable of much higher efficiency expression and scaffold multimer assembly than the wild-type RBD. Second, we have observed that RBD antigens express much more efficiently, and interfere less with the fidelity of multimer assembly, when fused to the C-terminus of the scaffold protein rather than the N-terminus.

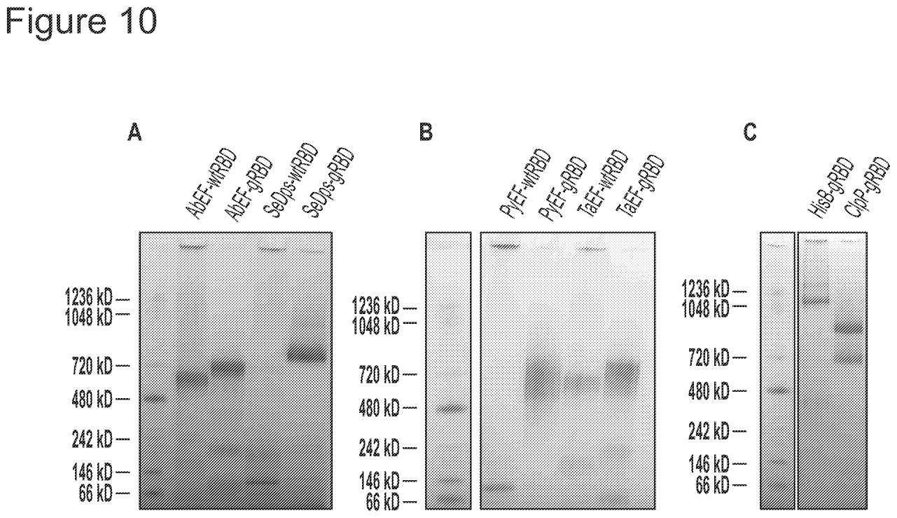

The observation that the fusion of the gRBD to the C-terminus of a scaffold multimer allowed efficient expression and particle assembly was extended to other scaffold proteins. The gRBD was fused to the C-terminus of bacterial encapsulated ferritin from Acidiferrobacteraceae bacterium (AbEF) and a Dps from Salmonella Enterica (FIG. 10A), archaeal encapsulated ferritins from Pyrococcus yayanosii (PyEF) and Thermoplasmata archaeon (TaEF) (FIG. 10B). Indeed, the gRBD expressed efficiently and assembled as a multimer for when fused to the C-terminus of AbEF, Dps, PyEF, and TaEF. Moreover, when C-terminal fusions of the wild-type RBD versus the gRBD were compared side-by-side in the context of AbEF Dps, PyEF, and TaEF, the multimers were generated more efficiently for the gRBD than the wild-type RBD. Indeed, the wild-type RBD did not allow the assembly of Dps or PyEF multimers at all, whereas the gRBD allowed efficient Dps and PyEF multimer assembly. Thus, the engineered glycans present in the gRBD enable its expression as a C-terminal fusion on many self-assembling multimer scaffolds.

### Example 5—Novel Families of Scaffolds Based on ClpP and HisB

Two novel families scaffolds were identified that have optimal properties, including an available C-terminus, and self-assembly into homo-multimers containing between 12 and 60. Specifically, ATP-dependent Clp protease proteolytic subunit ClpP (14-mer), and imidazoleglycerol-phosphate dehydratase HisB (24-mer). The sequences of numerous orthologs of HisB and ClpP are available in sequence databases. However, the HisB and ClpP proteins of Staphylococcus aureus (SaHisB and SaClpP) were chosen as examples. The gRBD was fused to the C-terminus of ClpP and HisB, expressed by transient transfection, and analyzed by native protein gel electrophoresis (FIG. 10C). Both ClpP-gRBD and HisB-gRBD expressed efficiently has self-assembling homo-multimers. However, native gel electrophoresis of ClpB-gRBD revealed that it assembled as 7-mers and 14-mers (i.e., halves and wholes) of the 14-mer multimer (FIG. 10C). By contrast, HisB-gRBD expressed with excellent fidelity as a 24-mer (FIG. 10C). It deserves special emphasis that an optimal outcome was observed, in that HisB multimers formed a single homogenous band on a native protein gel at the expected size for a 24-mer. Therefore, HisB proteins provide a high-fidelity self-assembling 24-mer scaffold. Furthermore, the wild-type RBD caused HisB to aggregate, even as a C-terminal fusion. Thus, ClpP and HisB provide novel scaffolds with optimal properties for expressing vaccine antigens, e.g., gRBD.

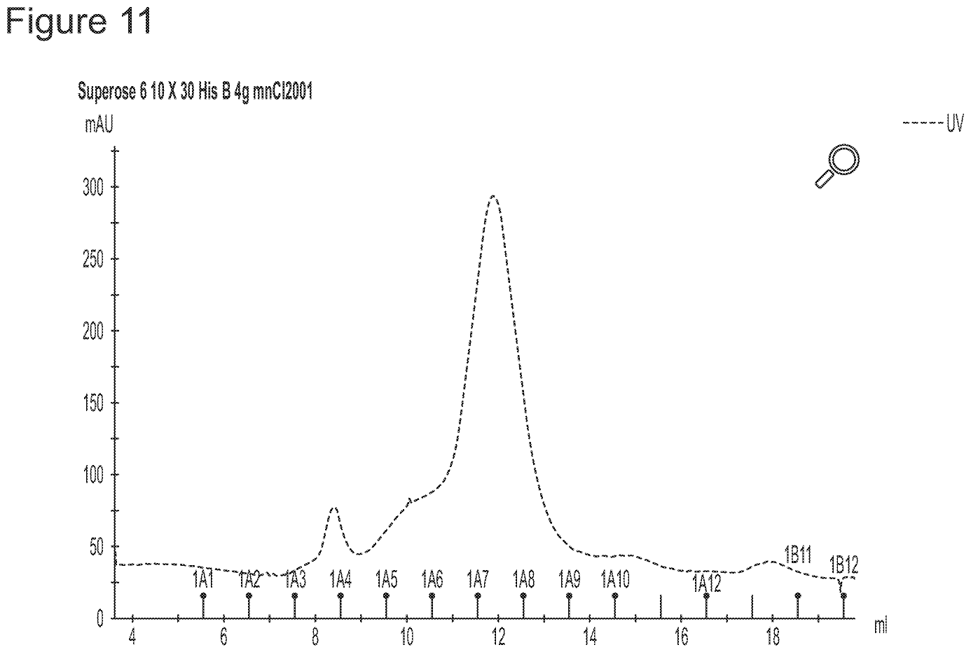

The HisB-gRBD fusion protein expressed efficiently as a single multimer peak that could be resolved by size-exclusion chromatography (SEC) (FIG. 11A). This single peak, when analyzed by native protein electrophoresis, was almost entirely a single band with the expected molecular weight for a 24-mer. Thus, HisB with an antigen fused to its C-terminus self-assembles with high fidelity.

Assembly of HisB trimers into the 24-mer requires coordination by Manganese ions (Sinha et al., J Biol Chem. 2004 Apr. 9; 279(15):15491-8). While this is not expected to affect assembly in vivo, where a low but consistent level of this trace metal in serum supports assembly, it is limiting in cell culture. We found a variable proportion of HisB-gRBD was purified in the form of a trimer, and that this trimer could be assembled into 24-mers by the addition of MnCl₂, but disassembly by incubation with EDTA was slow (FIG. 11B). This allows production and purification of trimers under conditions where Manganese is limiting, followed by Manganese-induced assembly. This would be of particular interest in yeast culture. Yeast is an attractive host for glycoprotein antigen production based on cost and safety, but the diffusion limit of the cell wall can be a bottleneck for larger proteins (Tang et al., Sci Rep. 2016 May 9; 6:25654). However, a number of proteins in the 100 kDa range have been produced to reasonable yield in yeast (Hung et al., Mol Cell Proteomics. 2016 Oct.; 15(10):3090-3106). Therefore, production of trimers in yeast cultured in the absence of Manganese, followed by purification and subsequent multimerization in the presence of Manganese, is a strategy for generating HisB multimers in yeast.

Additionally, the trimer is much more amenable to purification by conventional affinity media, where the capacity for nanoparticle purification is limited to the outermost fraction due to pore size constraints. Downstream processing could be greatly simplified by purification, followed by assembly with Mn²⁺ and polishing by Size Exclusion Chromatography, which can be used to separate separated particles from trimers.

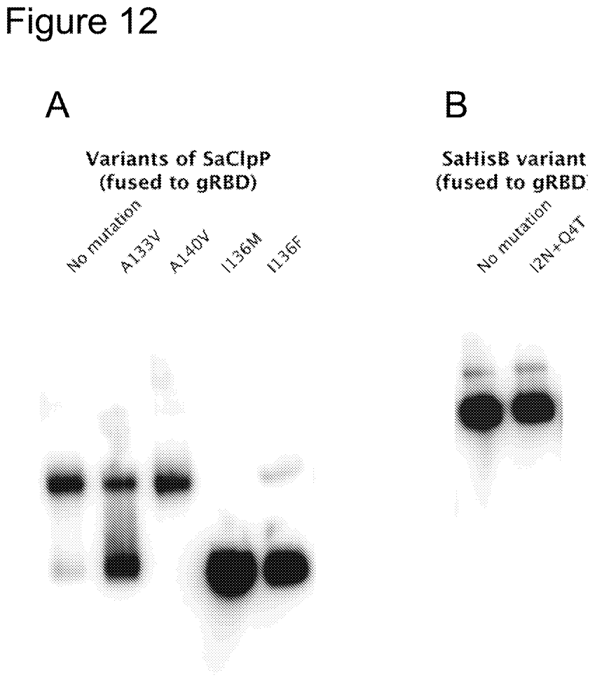

Building on the observation that S. aureus ClpP (SaClpP) initially generated a heterogeneous mixture of 7-mers and 14-mers, efforts were undertaken to improve the fidelity of ClpP multimer assembly. Several substitutions were engineered into SaClpP with the intention of stabilizing the conformation and/or interactions responsible for homo-multimerization, including A133V, A140V, 1136M, and 1136F of SEQ ID NO:97. Indeed, A140V greatly improved the fidelity of multimerization without any loss in yield (FIG. 12A). Thus, A140V enables the high-fidelity production of ClpP 14-mers as a vaccine antigen scaffold.

The substitutions A133V, A140V, I136M, and I136F were selected based on the approach of filling empty spaces within hydrophobic regions of the protein or multimer, by replacing a hydrophobic amino acid with a different hydrophobic amino acid of greater number of carbon atoms or molecular weight than the one being replaced.

In the context of scaffold-display of vaccine antigens, one advantageous feature of the strategy of engineering glycans onto the RBD of SARS-CoV-2 is the engineered glycans have the potential to partially occlude the scaffold, and thereby focus the antibody response onto the antigen and away from the scaffold. The HisB of S. aureus also contains an NX(S/T) motif for N-linked glycosylation at position N15 of SEQ ID NO:34 that is glycosylated when it is expressed in mammalian cells (although proteins are not glycosylated at NX(S/T) motifs in bacteria). To further advance the feature of the gRBD and the S. aureus HisB that they may partially occlude the scaffold with N-linked glycans, an additional N-linked glycan was engineered onto the HisB scaffold through the substitutions 12N/Q4T, relative to the amino acid number of S. aureus HisB (SEQ ID NO:34). Importantly, the introduction of this N-linked glycan through the substitutions I2N/Q4T did not affect multimerization fidelity or yield (FIG. 12B). Together with the engineered glycans of the gRBD, the I2N/Q4T glycan helps to create a glycan shield around the scaffold that focuses the immune response onto the antigen.

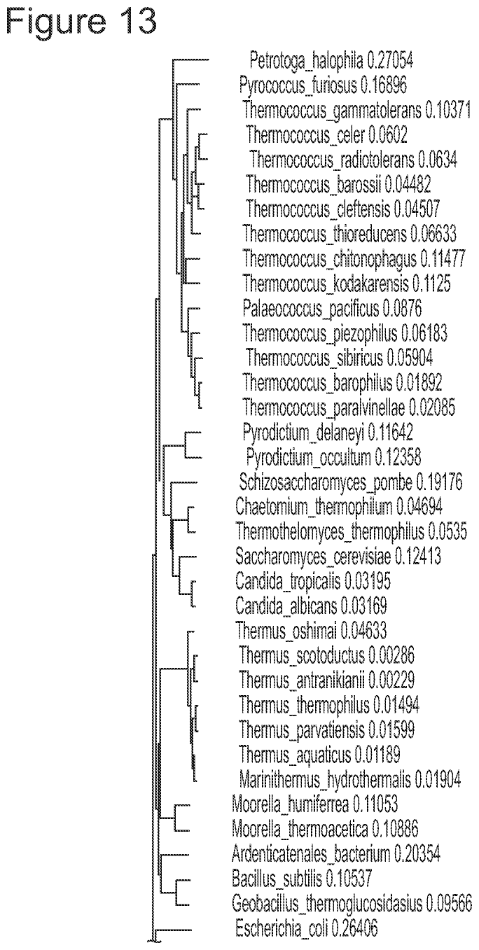

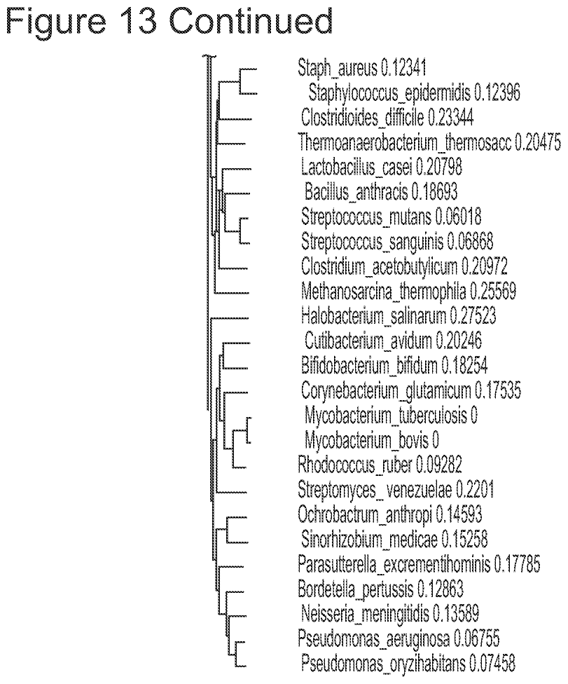

Due to the optimal properties of HisB and ClpP, sequence data was analyzed for diverse HisB and ClpP proteins. HisB proteins from bacteria including human commensals, human pathogens, thermophiles, and hyperthermophiles, from archaea including mesophiles, thermophiles, and hyperthermophiles, and from fungi including human commensals, human pathogens, mesophiles, and thermophiles were analyzed (SEQ ID NOs:34-96). To facilitate the selection of diverse sequences, and the grouping of sequences to identify multi-species consensus sequences, a phylogenetic tree was constructed of HisB orthologs (FIG. 13). An antigen, e.g., the gRBD, can be fused to the C-terminus of these HisB orthologs or modified variants thereof to generate a self-assembling homo-multimer immunogen for a vaccine.

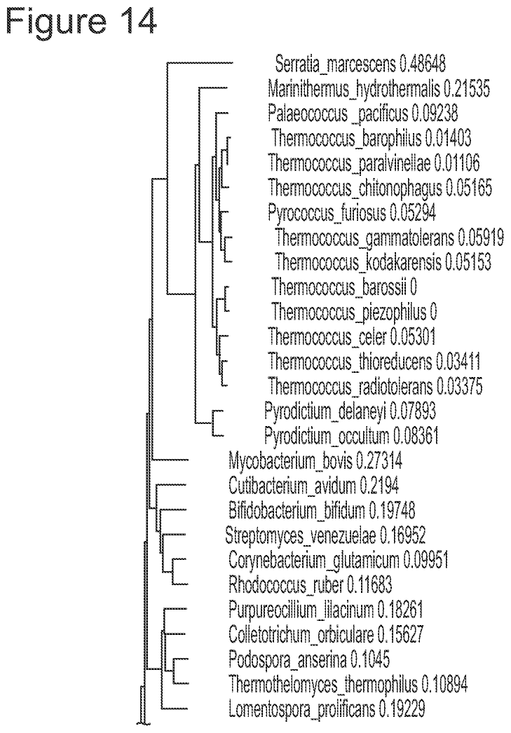

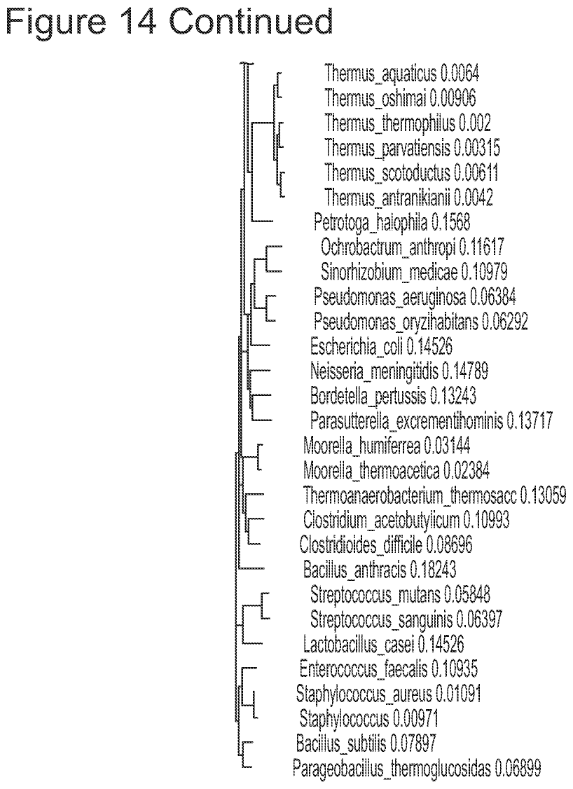

Likewise, ClpP proteins from bacteria including human commensals, human pathogens, thermophiles, and hyperthermophiles, from archaea including thermophiles and hyperthermophiles, and from fungi including mesophiles, fungi capable of causing opportunistic infections in humans, and thermophiles were analyzed (SEQ ID NO:97-154). To facilitate the selection of diverse sequences, and the grouping of sequences to identify multi-species consensus sequences, a phylogenetic tree was constructed of ClpP orthologs (FIG. 14). An antigen, e.g., the gRBD, can be fused to the C-terminus of these ClpP orthologs or modified variants thereof to generate a self-assembling homo-multimer immunogen for a vaccine.

Observations using scaffolds evaluated and described hereunder are summarized in Table 1.

TABLE 1

| | | | | | | N-term | | C-term | | | | |
|---|---|---|---|---|---|---|---|---|---|---|---|---|
| | | #- | | | Available | yield | N-term % | yield | C-term % | | | |
| Platform | Gene family | mer | Species | Environment | termini | (mg/L) | multimer | (mg/L) | multimer | Accession | Construction | Observations |
| Hp-NAP | Dps | 12 | Helicobacter | Mesophile | Both | 117 | 15% | 100 | 95% | WP_000846479 | No mutations | Dominant 24mer, |
| | | | pylori | | | | | | | | | some 2-particle |
| | | | | | | | | | | | | doublet.. Some |
| | | | | | | | | | | | | monomer |
| Sc-Dps | Dps | 12 | Salmonella | Mesophile | Both | | | 73 | 95% | EBN4514793 | aa 12-167, | Dominant 24mer, |
| | | | enterica | | | | | | | | DNA binding | some 2-particle |
| | | | | | | | | | | | N-terminus | doublet. Some |
| | | | | | | | | | | | not used | monomer |
| Li-Dps | Dps | 12 | Listeria | Mesophile | Both | | | 68 | 80% | WP_185504746 | N81Q | Dominant 24mer, |
| | | | innocua | | | | | | | | | some 2-particle |
| | | | | | | | | | | | | doublet. No |
| | | | | | | | | | | | | monomer |
| Mt-DoD | Dodecin | 12 | Mycobacterium | Mesophile | Both | 57 | 0% | | | WP_003898900 | No mutations | N-terminal fusion |
| | | | tuberculosis | | | | | | | | | is apparent trimers |
| Bp-DoD | Dodecin | 12 | Bordetella | Mesophile | Both | 68 | 5% | 42 | 95% | WP_010930433 | aa 2-71 | N-terminal fusion |
| | | | pertussis | | | | | | | | | is apparent trimers |
| Ap-ENcFtn | Encapsulated | 10 | Acidiferro- | Thermophile | C | | | 96 | 90% | HEC13526 | C44A | No aggregate, 5% |
| | Ferritin | | bacteraceae | | | | | | | | | subassembly, 5% |
| | | | bacterium | | | | | | | | | monomer |
| Py-EncFtn | Encapsulated | 10 | Pyrococcus | Thermophile | C | | | 55 | 85% | WP_048058214 | No mutations | Low aggregate, |
| | Ferritin | | yayanosii | | | | | | | | | no subassembly, |
| | | | | | | | | | | | | some monomer |
| Ta-EncFtn | Encapsulated | 10 | Thermoplasmata | Thermophile | C | | | 64 | 90% | RLF66362 | No mutation | No aggregate, 5% |
| | Ferritin | | archaeon | | | | | | | | | subassembly, 5% |
| | | | | | | | | | | | | monomer |
| Hp-ferritin | Ferritin | 24 | Helicobacter | Mesophile | N | 12 | 40% | | | WP_000949190 | aa 5-167, | some aggregate, |
| | | | pylori | | | | | | | | S21A, C31A | some monomer |
| | | | | | | | | | | | Tween-20 | |
| | | | | | | | | | | | prior to | |
| | | | | | | | | | | | filtration for | |
| | | | | | | | | | | | purification. | |
| HP-ferritin | Ferritin | 24 | Helicobacter | Mesophile | N | 19 | 20% | | | WP_000949190 | aa 1-167, | Some aggregate, as |
| v2 | | | pylori | | | | | | | | S21A, C31A. | much monomer as |
| | | | | | | | | | | | No tween-20 | multimer |
| | | | | | | | | | | | required. | |
| dE-ferritin | Ferritin | 24 | Helicobacter | Mesophile | Both | | | 4 | 20% | WP_000949190 | aa 1-144, | Aggregated, |
| | | | pylori | | | | | | | | S21A, C31A. | dominant smear, |
| | | | | | | | | | | | Deleted E | subassembly (2) |
| | | | | | | | | | | | helix | and monomer |
| Aa-LS | Lumazine | 60 | Aquifex | Thermophile | Both | | | 5 | 0% | WP_010880027 | C37A, | Aggregate, slight |
| | synthase | | aeolicus | | | | | | | | N102D | smear |
| mi3 | KDPG | | | | | | | | | | | |
| | aldolase | 60 | Thermotoga | Thermophile | N | 11 | 20% | | | AXF54357 | mi3 is | High aggregate, |
| | | | maritima | | | | | | | | cysteine | some monomer. |
| | | | | | | | | | | | mutant | Extra band at 1 |
| | | | | | | | | | | | version | MDa not on |
| | | | | | | | | | | | of i3-01 | native Western |
| | | | | | | | | | | | | Blot |
| MjHsp16.5 | Small heat | 24 | Methanocal- | Thermophile | C | | | 58 | 0% | WP_010869783 | aa 33-147 | some aggregate, |
| | shock protein | | dococcus | | | | | | | | | mostly dimer(80%) |
| | | | jannaschii | | | | | | | | | and hexamer(10%) |
| EcYfbU | hypothetical | 24 | E.coli | Mesophile | C | | | 21 | 95% | WP_096981428 | C65S, C153A | Fuzzy band, very |
| | protein | | | | | | | | | | | low aggregate, |
| | | | | | | | | | | | | some half (12mer), |
| | | | | | | | | | | | | no monomer |
| Sa-ClpP | ClpP | 14 | Staphylococcus | Mesophile | C | | | 97 | 75% | WP_001049165 | C92A, L144R | No aggregate, |
| | | | aureus | | | | | | | | | some heptamers |
| | | | | | | | | | | | | few monomers |
| Sa-HisB | IGPD | 24 | Staphylococcus | Mesophile | C | | | 60 | 75% | AFH70952 | S118A | low aggregate, |
| | | | aureus | | | | | | | | | some 2-particle |
| | | | | | | | | | | | | doublet, no |
| | | | | | | | | | | | | monomers, trimers |
| | | | | | | | | | | | | only when Mn2+ |
| | | | | | | | | | | | | is limiting |

### Example 6—RBD Antigens Based on Naturally-Occurring Variants of SARS-CoV-2

Glycosylation sites may be engineered onto naturally-occurring variants of the RBD of SARS-CoV-2.

For instance, the naturally-occurring SARS-CoV-2 RBD sequence has the RBD sequence:

(SEQ ID NO: 155)
NITNLCPFGEVFNATRFASVYAWNRKRISNCVADYSVLYN
SASFSTFKCYGVSPTKLNDLCFTNVYADSFVIRGDEVRQI
APGQTGKIADYNYKLPDDFTGCVIAWNSNNLDSKVGGNYN
YLYRLFRKSNLKPFERDISTEIYQAGSTPCNGVEGFNCYF
PLQSYGFQPTNGVGYQPYRVVVLSFELLHAPATVCGP

A gRBD variant based on this naturally-occurring SARS-CoV-2 sequence, containing the four engineered N-linked glycans, has the sequence:

(SEQ ID NO: 162)
NITNLCPFGEVFNATRFASVYAWNRKRISNCVADYSVLYN
STSFSTFKCYGVSPTKLNDLCFTNVTADSFVIRGDEVRQI
APGQTGKIADYNYKLPDNFTGCVIAWNSNNLDSKVGGNYN
YLYRLFRKSNLKPFERDISTEIYQAGSTPCNGVEGENCYF
PLQSYGFQPTNGVGYQPYRVVVLSFENLTAPATVCGP

A naturally-occurring SARS-CoV-2 RBD sequence known as the UK variant, B.1.1.7, and “Alpha” lineage has the sequence:

(SEQ ID NO: 156)
NITNLCPFGEVFNATRFASVYAWNRKRISNCVADYSVLYNSASFSTFKC
YGVSPTKLNDLCFTNVYADSFVIRGDEVRQIAPGQTGKIADYNYKLPDD
FTGCVIAWNSNNLDSKVGGNYNYLYRLFRKSNLKPFERDISTEIYQAGS
TPCNGVEGFNCYFPLQSYGFQPTYGVGYQPYRVVVLSFELLHAPATVCG
P.

A gRBD variant based on the naturally-occurring SARS-CoV-2 RBD sequence of SEQ ID NO:156 has the sequence:

(SEQ ID NO: 163)
NITNLCPFGEVFNATRFASVYAWNRKRISNCVADYSVLYNSASFSTFKC
YGVSPTNLSDLCFTNVYADSFVIRGDEVRQIAPGQTGKIADYNYKLPDN
FTGCVIAWNSNNLDSKVGGNYNYLYRLFRKSNLKPFERDISTEIYQAGS
TPCNGVEGENCYFPLQSYGFQPTYGVGYQPYRVVVLSFENGTNGTTVCG
P.

A naturally-occurring SARS-CoV-2 RBD sequence known as the California variant, B.1.429, and “Epsilon” lineage has the sequence:

(SEQ ID NO: 157)
NITNLCPFGEVFNATRFASVYAWNRKRISNCVADYSVLYNSASFSTFKC
YGVSPTKLNDLCFTNVYADSFVIRGDEVRQIAPGQTGKIADYNYKLPDD
FTGCVIAWNSNNLDSKVGGNYNYRYRLFRKSNLKPFERDISTEIYQAGS
TPCNGVEGFNCYFPLQSYGFQPTNGVGYQPYRVVVLSFELLHAPATVCG
P.

A gRBD variant based on the naturally-occurring SARS-CoV-2 RBD sequence of SEQ ID NO:157 has the sequence:

(SEQ ID NO: 164)
NITNLCPFGEVFNATRFASVYAWNRKRISNCVADYSVLYNSASFSTFKC
YGVSPTNLSDLCFTNVYADSFVIRGDEVRQIAPGQTGKIADYNYKLPDN
FTGCVIAWNSNNLDSKVGGNYNYRYRLFRKSNLKPFERDISTEIYQAGS
TPCNGVEGENCYFPLQSYGFQPTNGVGYQPYRVVVLSFENGTNGTTVCG
P.

A naturally-occurring SARS-CoV-2 RBD sequence known as the South Africa variant, B.1.351, and “Beta” lineage has the sequence:

(SEQ ID NO: 158)
NITNLCPFGEVFNATRFASVYAWNRKRISNCVADYSVLYNSASFSTFKC
YGVSPTKLNDLCFTNVYADSFVIRGDEVRQIAPGQTGNIADYNYKLPDD
FTGCVIAWNSNNLDSKVGGNYNYLYRLFRKSNLKPFERDISTEIYQAGS
TPCNGVKGFNCYFPLQSYGFQPTYGVGYQPYRVVVLSFELLHAPATVCG
P.

A gRBD variant based on the naturally-occurring SARS-CoV-2 RBD sequence of SEQ ID NO:158 has the sequence:

(SEQ ID NO: 165)
NITNLCPFGEVFNATRFASVYAWNRKRISNCVADYSVLYNSASFSTFKC
YGVSPTNLSDLCFTNVYADSFVIRGDEVRQIAPGQTGNIADYNYKLPDN
FTGCVIAWNSNNLDSKVGGNYNYLYRLFRKSNLKPFERDISTEIYQAGS
TPCNGVKGFNCYFPLQSYGFQPTYGVGYQPYRVVVLSFENGTNGTTVCG
P.

A naturally-occurring SARS-CoV-2 RBD sequence known as the Brazil variant, P.1, and “Gamma” lineage has the sequence:

(SEQ ID NO: 159)
NITNLCPFGEVFNATRFASVYAWNRKRISNCVADYSVLYNSASFSTFKC
YGVSPTKLNDLCFTNVYADSFVIRGDEVRQIAPGQTGTIADYNYKLPDD
FTGCVIAWNSNNLDSKVGGNYNYLYRLFRKSNLKPFERDISTEIYQAGS
TPCNGVKGFNCYFPLQSYGFQPTYGVGYQPYRVVVLSFELLHAPATVCG
P.

A gRBD variant based on the naturally-occurring SARS-CoV-2 RBD sequence of SEQ ID NO:159 has the sequence:

(SEQ ID NO: 166)
NITNLCPFGEVFNATRFASVYAWNRKRISNCVADYSVLYNSASFSTFKC
YGVSPTNLSDLCFTNVYADSFVIRGDEVRQIAPGQTGTIADYNYKLPDN
FTGCVIAWNSNNLDSKVGGNYNYLYRLFRKSNLKPFERDISTEIYQAGS
TPCNGVKGFNCYFPLQSYGFQPTYGVGYQPYRVVVLSFENGTNGTTVCG
P.

A naturally-occurring SARS-CoV-2 RBD sequence known as the India variant, B.1.617.2, and “Delta” lineage has the sequence:

(SEQ ID NO: 160)
NITNLCPFGEVFNATRFASVYAWNRKRISNCVADYSVLYNSASFSTFKC
YGVSPTKLNDLCFTNVYADSFVIRGDEVRQIAPGQTGKIADYNYKLPDD
FTGCVIAWNSNNLDSKVGGNYNYRYRLFRKSNLKPFERDISTEIYQAGS
KPCNGVEGENCYFPLQSYGFQPTNGVGYQPYRVVVLSFELLHAPATVCG
P.

A gRBD variant based on the naturally-occurring SARS-CoV-2 RBD sequence of SEQ ID NO:160 has the sequence:

(SEQ ID NO: 167)
NITNLCPFGEVFNATRFASVYAWNRKRISNCVADYSVLYNSASFSTFKC
YGVSPTNLSDLCFTNVYADSFVIRGDEVRQIAPGQTGKIADYNYKLPDN
FTGCVIAWNSNNLDSKVGGNYNYRYRLFRKSNLKPFERDISTEIYQAGS
KPCNGVEGENCYFPLQSYGFQPTNGVGYQPYRVVVLSFENGTNGTTVCG
P.

A naturally-occurring SARS-CoV-2 RBD sequence known as the India variant, B.1.617.1, and “Kappa” lineage has the sequence:

(SEQ ID NO: 161)
NITNLCPFGEVFNATRFASVYAWNRKRISNCVADYSVLYNSASFSTFKC
YGVSPTKLNDLCFTNVYADSFVIRGDEVRQIAPGQTGKIADYNYKLPDD
FTGCVIAWNSNNLDSKVGGNYNYRYRLFRKSNLKPFERDISTEIYQAGS
TPCNGVQGFNCYFPLQSYGFQPTNGVGYQPYRVVVLSFELLHAPATVCG
P.

A gRBD variant based on the naturally-occurring SARS-CoV-2 RBD sequence of SEQ ID NO:161 has the sequence:

(SEQ ID NO: 168)
NITNLCPFGEVFNATRFASVYAWNRKRISNCVADYSVLYNSASFSTFKC
YGVSPTNLSDLCFTNVYADSFVIRGDEVRQIAPGQTGKIADYNYKLPDN
FTGCVIAWNSNNLDSKVGGNYNYRYRLFRKSNLKPFERDISTEIYQAGS
TPCNGVQGFNCYFPLQSYGFQPTNGVGYQPYRVVVLSFENGTNGTTVCG
P.

Such naturally-occurring sequences may be advantageous due to matching the sequences of emerging viral variants, and/or possessing other features that were positively selected in viral evolution, e.g., improved expression. Versions of the gRBD and fusion proteins thereof, e.g., containing scaffold proteins, can be engineered from emerging viral variants.

Such naturally-occurring sequences are described in additional detail in Table 2. gRBDs and multimers thereof containing the substitutions enumerated in Table 2 are useful for eliciting antibodies directed against the variant epitopes, and/or focusing antibody responses away from the variant epitopes.

TABLE 2

| | | Commonly used names for source |
|---|---|---|
| Suffix | RBD mutations | viruses |
| No | None | “Wuhan variant”, WIV04/2019, 2019 |
| suffix | | variants, Reference sequence. Index. |
| -alpha | N501Y | “UK variant”, B.1.1.7, Alpha |
| -beta | K417N, E484K, N501Y | “South African variant”, B.1351, |
| | | Beta |
| -gamma | K417T, E484K, N501Y | “Brazil variant”, P.1, B.1.1.248, |
| | | Gamma |
| -delta | L452R, T478K | “Indian 2 variant”, B.1.617.2, Delta |
| -epsilon | L452R, E484Q* | “California variants”, B1.427/B1.429, |
| | | Epsilon; *E484 only in B1.429 |
| -zeta | E484K | P.2, also from Brazil, Zeta |
| -eta | N439K, E484K | B.1.525, also from UK, Eta |
| -theta | E484K, N501Y | P.3, from Philippines, Theta |
| -iota | S477N or E484K | “New York variant”, B 1.526, Iota |
| -kappa | L452R, E484Q | “Indian 1 variant”, B.1.617.1, Kappa |

As exemplified with SEQ ID NOs:3, 162-168 and 241-246, N-linked glycans can be engineered into corresponding naturally-occurring RBD sequences (SEQ ID NOs:2 and 155-161) to generate “gRBDs” with improved solubility and aggregation particularly when expressed as multimers. Notably, naturally-occurring substitutions can be mixed-and-matched, i.e., swapped, among different RBDs to generate chimeric RBDs, and stabilizing glycans can be engineered into chimeric RBDs as well. Glycans were engineered into positions 370, 386, 394, 428, 517, and/or 520 (with respect to the reference sequence numbering, SEQ ID NO:1) (Table 3). Seven combinations of these substitutions were designated gRBD.1-gRBD.7 (Table 3). It was noted that gRBD.5 was the best expressing, and most immunogenic in the Beta variant. It was further noted that gRBD.6 and gRBD.7 were highly expressing in the context of the Reference strain, Alpha/UK, Beta/South Africa, and Delta/India variants (Table 3).

TABLE 3
Substitutions in the RBDs of variants of SARS-CoV-2

| | Engineered glycan | |
|---|---|---|
| Prefix | positions | Comments |
| gRBD.1 | 370, 394, 428, 517 | Most immunogenic with Reference |
| | | sequence and Alpha. |
| gRBD.2 | 370, 428, 517 | |
| gRBD.3 | 386, 428, 517 | |
| gRBD.4 | 386, 428, 517, 520 | |
| gRBD.5 | 370, 428, 517, 520 | Best expressing, most immunogenic with |
| | | Beta |
| gRBD.6 | 360, 370, 428, 517 | High expression with Reference, Alpha, |
| | | Beta, Delta |
| gRBD.7 | 360, 370, 428, 517, | High expression with Reference, Alpha, |
| | 520 | Beta, Delta |

The amino acid sequences of these RBD variants are shown below in SEQ ID NOs:3 and 241-246, respectively. Residues in italics denote glycosylations, and underlined residues correspond to sites of mutations.

gRBD.1
(SEQ ID NO: 3)
NITNLCPFGEVFNATRFASVYAWNRKRISNCVADYSVLYNSTSFSTFKC
YGVSPTKLNDLCFTNVTADSFVIRGDEVRQIAPGQTGKIADYNYKLPDN
FTGCVIAWNSNNLDSKVGGNYNYLYRLFRKSNLKPFERDISTEIYQAGS
TPCNGVEGENCYFPLQSYGFQPTNGVGYQPYRVVVLSFENLTAPATVCG
P
gRBD.2
(SEQ ID NO: 241)
NITNLCPFGEVFNATRFASVYAWNRKRISNCVADYSVLYNSTSFSTFKC
YGVSPTKLNDLCFTNVYADSFVIRGDEVRQIAPGQTGKIADYNYKLPDN
FTGCVIAWNSNNLDSKVGGNYNYLYRLFRKSNLKPFERDISTEIYQAGS
TPCNGVEGENCYFPLQSYGFQPTNGVGYQPYRVVVLSFENLTAPATVCG
P
gRBD.3
(SEQ ID NO: 242)
NITNLCPFGEVFNATRFASVYAWNRKRISNCVADYSVLYNSASFSTFKC
YGVSPTNLTDLCFTNVYADSFVIRGDEVRQIAPGQTGKIADYNYKLPDN
FTGCVIAWNSNNLDSKVGGNYNYLYRLFRKSNLKPFERDISTEIYQAGS
TPCNGVEGENCYFPLQSYGFQPTNGVGYQPYRVVVLSFENLTAPATVCG
P
gRBD.4
(SEQ ID NO: 243)
NITNLCPFGEVFNATRFASVYAWNRKRISNCVADYSVLYNSASFSTFKC
YGVSPTNLTDLCFTNVYADSFVIRGDEVRQIAPGQTGKIADYNYKLPDN
FTGCVIAWNSNNLDSKVGGNYNYLYRLFRKSNLKPFERDISTEIYQAGS
TPCNGVEGENCYFPLQSYGFQPTNGVGYQPYRVVVLSFENGTNGTTVCG
P
gRBD.5
(SEQ ID NO: 244)
NITNLCPFGEVFNATRFASVYAWNRKRISNCVADYSVLYNSTSFSTFKC
YGVSPTKLNDLCFTNVYADSFVIRGDEVRQIAPGQTGKIADYNYKLPDN
FTGCVIAWNSNNLDSKVGGNYNYLYRLFRKSNLKPFERDISTEIYQAGS
TPCNGVEGENCYFPLQSYGFQPTNGVGYQPYRVVVLSFENGTNGTTVCG
P
gRBD.6
(SEQ ID NO: 245)
NITNLCPFGEVFNATRFASVYAWNRKRISNCTADYSVLYNSTSFSTFKC
YGVSPTKLNDLCFTNVYADSFVIRGDEVRQIAPGQTGKIADYNYKLPDN
FTGCVIAWNSNNLDSKVGGNYNYLYRLFRKSNLKPFERDISTEIYQAGS
TPCNGVEGFNCYFPLQSYGFQPTNGVGYQPYRVVVLSFENLTAPATVCG
P
gRBD.7
(SEQ ID NO: 246)
NITNLCPFGEVFNATRFASVYAWNRKRISNCTADYSVLYNSTSFSTFKC
YGVSPTKLNDLCFTNVYADSFVIRGDEVRQIAPGQTGKIADYNYKLPDN
FTGCVIAWNSNNLDSKVGGNYNYLYRLFRKSNLKPFERDISTEIYQAGS
TPCNGVEGFNCYFPLQSYGFQPTNGVGYQPYRVVVLSFENGTNGTTVCG
P

### Example 7—Scaffolds Based on Acidiferrobacteraceae Bacterium (Ap) Half-Ferritin

The half-ferritin of Acidiferrobacteraceae bacterium (Ap) (SEQ ID NO:10) was evaluated as a vaccine antigen scaffold. The sequence for this half-ferritin was derived from accession number HEC13526 (Table 1), which was deposited by Zhou et al., mSystems 5 (1), e00795-19 (2020), in a study titled “Genome- and Community- Level Interaction Insights into Carbon Utilization and Element Cycling Functions of Hydrothermarchaeota in Hydrothermal Sediment. This sequence was selected due to it being derived from a thermophile. The F10-gRBD fusion protein, where the N-terminus of the gRBD antigen was fused to the C-terminus of the 10-subunit Ap half-ferritin “F10” was noted to be one of the highest-expressing scaffolds (expressing at 96 mg/L by transient transfection), having excellent homogeneity expressing as 90% multimer, and have no aggregate formation (Table 1). Just 5% of the protein was observed to be monomer (Table 1). Based on these observations, F10 was selected for further evaluation and development.

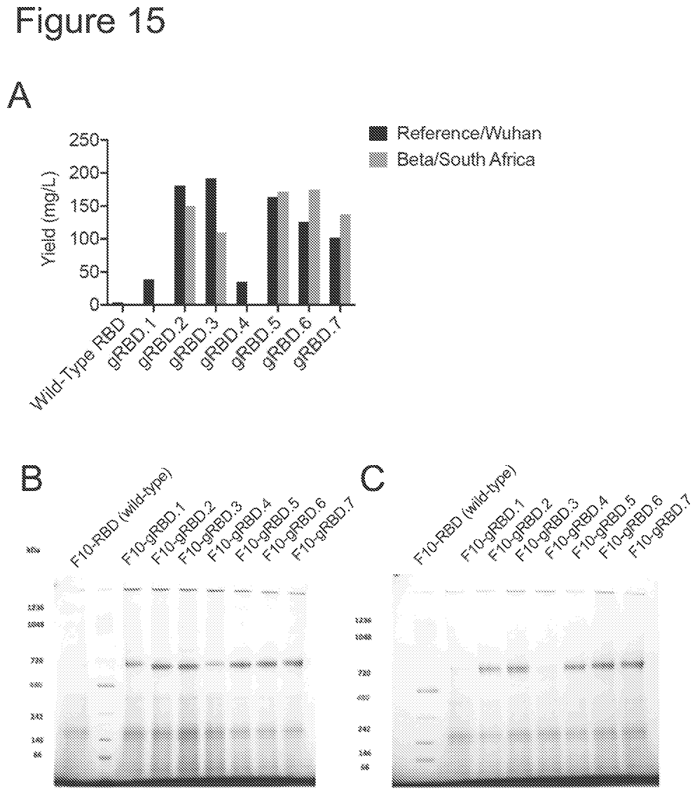

F10-gRBD fusion proteins expressed with excellent yields. F10-RBD and F10-gRBD fusions were cloned that were based on the Reference/Wuhan RBD sequence (SEQ ID NO:1) or the Beta/South Africa RBD sequence (SEQ ID NO:158). F10-gRBD sequences were derived containing the combinations of engineered glycans designated gRBD.1, gRBD.2, gRBD.3, gRBD.4, gRBD.5, gRBD.6, and gRBD.7, as indicated in Table 3. Plasmids encoding these F10-gRBD fusions, or an F10-RBD with the wild-type Reference/Wuhan control RBD, were transfected into Expi293 cells. F10-gRBD proteins were generated at excellent yields for transient transfection, between 100 and 200 mg/L, for F10-gRBD.2, F10-gRBD.3, and F10-gRBD.5-7 (FIG. 15A & Table 4). By contrast, the F10-RBD (with the unmodified wild-type Reference/Wuhan RBD sequence) was comparatively poorly expressed, yielding just mg/L. The combination of a modified gRBD and an F10 scaffold expressed efficiently as a fusion protein.

TABLE 4
Yields from Expi293 transfections to make F10-gRBD.1-7 fusions

Yields (mg/L)

| RBD fused to F10 | Reference/Wuhan | Beta/South Africa |
|---|---|---|
| Wild-Type RBD | 3.5 | Not Done |
| gRBD.1 | 38.7 | 0 |
| gRBD.2 | 180 | 149.7 |
| gRBD.3 | 191.4 | 109.5 |
| gRBD.4 | 34.7 | 1.2 |
| gRBD.5 | 163.2 | 171.3 |
| gRBD.6 | 125.6 | 174 |
| gRBD.7 | 101.8 | 136.9 |

These F10-gRBD fusions are self-assembling multimers. Unpurified cell culture supernatants from the Expi293 cell transfection described in FIG. 15A and Table 4 were analyzed by native gel electrophoresis, to assess multimerization. With the exception of the F10-RBD based on wild-type sequences, and gRBD.4, both the Reference/Wuhan (FIG. 15B) and Beta/South Africa (FIG. 15C) sets of F10-gRBD fusion proteins expressed mostly as multimers having the expected molecular weight for a 10-mer of 720 kDa on a native protein gel. These data show that the F10-gRBD fusion protein is a self-assembling multimer, which assembles with excellent fidelity.

These data also underscore the importance of the engineered glycans present in gRBD.1-gRBD.3 and gRBD.5-gRBD.7. The specific combinations of engineered glycans presented in these gRBDs are demonstrated in FIG. 15 and Table 4 to be optimal for the generation of engineered RBD multimers. Those specific combinations of engineered glycans are those at positions 370, 394, 428, 517 (gRBD.1), 370, 428, 517 (gRBD.2), 386, 428, 517 (gRBD.3), 370, 428, 517, 520 (gRBD.5), 360, 370, 428, 517 (gRBD.6), and 360, 370, 428, 517, 520 (gRBD.7).

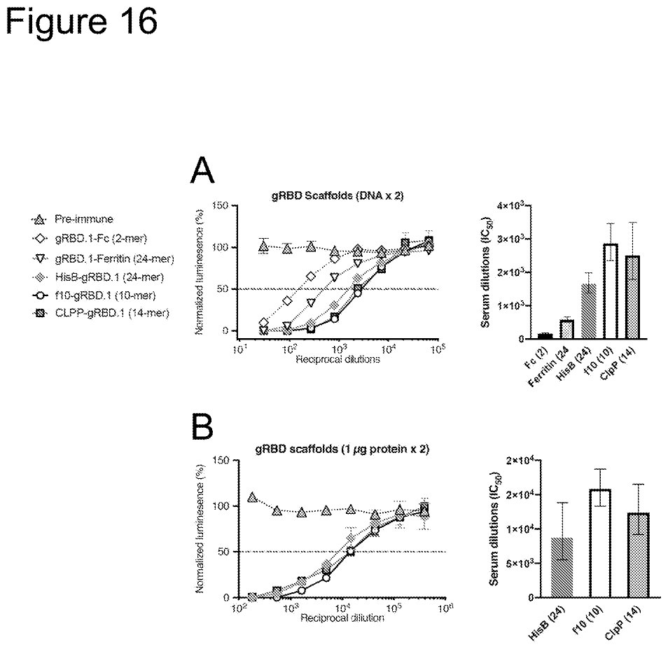

Ap half-ferritin (F10) was compared against other scaffolds in comparative vaccine immunogenicity studies in mice. In a first experiment, an antibody Fc (dimer), a whole or classical ferritin (24-mer), HisB (48-mer), ClpP (14-mer), and the Ap half-ferritin F10 (10-mer) were compared for immunogenicity after intramuscular electroporation of a plasmid DNA encoding a fusion protein of a gRBD antigen and the scaffold protein in mice. The mice were electroporated gastrocnemius muscle with 60 μg DNA on days 0 and 14. Serum was collected on day 21 and pooled for neutralization assays. F10-gRBD elicited the most potent neutralizing antibodies, neutralizing 50% of SARS-CoV-2 pseudovirus infection at a titer of approximately 1:3,000 (FIG. 16A). This titer was a significant improvement over that elicited by the 24-mer ferritin, which elicited neutralizing antibodies with a titer of approximately 1:600 (FIG. 16A and FIGS. 7D&H). The neutralizing antibody titers elicited in this experimented pointed to F10 as an optimal scaffold for antigen presentation.

The ability of a scaffold-antigen fusion protein to express in a manner that is presented in a manner such that antibody induction is efficient is controlled for by DNA electroporation. In a DNA electroporation study, one of the variables among experimental conditions is expression efficiency, in a manner that can ultimately interact efficiently with B cells. DNA electroporation is like other platforms for expression in vivo from a nucleic acid, e.g., an mRNA or modified mRNA. Thus, the results of DNA electroporation studies directly inform which antigens and scaffolds will perform well in mRNA delivery approaches.

To control for differences in expression, mice also were immunized with normalized amounts of recombinant protein. The immunogenicity of three novel scaffolds disclosed herein, HisB, ClpP, and F10, were compared as fusion proteins with gRBD antigens, in the context of recombinant protein. Mice were inoculated twice weekly with 1 μg of protein antigen formulated with 5 μg QuilA and MPLA adjuvants. Normalized for the recombinant protein input, the neutralization titers elicited in mice were similar (FIG. 16B). However, F10-gRBD elicited the most potent neutralizing antibody titers, with a rank order from most-to-least potent of F10-gRBD>ClpP-gRBD>HisB-gRBD.

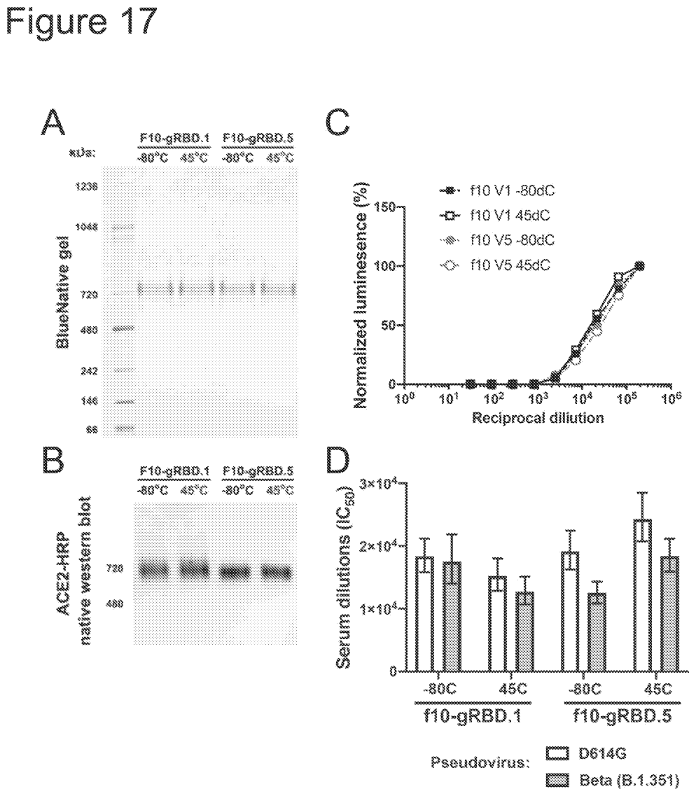

F10-gRBD can be freeze-dried and retains full immunogenicity after reconstitution. F10 and all gRBD versions have been selected for thermal stability, and F10 derives from a prokaryotic thermophile, raising the possibility that an F10-gRBD fusion protein multimer would be sufficiently stable to lyophilize and reconstitute to full activity. To evaluate this possibility, F10-gRBD.1 and F10-gRBD.5 were freeze dried in 0.5M trehalose, a sugar commonly used as a lyoprotectant. Freeze-dried antigens were either frozen at −80° C. or heat-stressed for 48 hours at 45° C. (113° F.). These materials were then reconstituted in PBS and analyzed by native gel electrophoresis (FIG. 17A) and by native western blotting with HRP-conjugated ACE2 (FIG. 17). Strikingly, F10.gRBD.1 and F10.gRBD.5 fully maintained their structure after significant heat stress, as indicated by the fact that all visible material ran at 720 kDa, the sized of the assembled 10-mer. Moreover, there did not appear to be any loss of ACE2 binding, as indicated by the native western blot with ACE2-Fc. Consistent with these observations, immunization of mice (5 per group) with each of these antigens raised very similar and potent neutralizing sera, essentially identical with that observed with the same antigen maintained in the liquid state (see FIG. 16B for example). These results show that F10-gRBD vaccines are particularly useful, with respect to their ability to be lyophilized, transported without a consistent cold chain, and retain their immunogenicity upon reconstitution.

The ability of the baculovirus/Sf9 cell system to express F10-gRBD was explored due to several potential advantages of the baculovirus/Sf6 system in vaccine generation. These advantages include the availability of Sf9 cell lines that are compliant with current good manufacturing practice (cGMP) use, for generation of material to be used in humans. Whereas many other cell culture systems require obtaining a new cell line specifically for each antigen, the baculovirus/Sf9 system merely requires the generation and banking of baculovirus stocks, which are they used to inoculate a cGMP-compatible Sf9 cell line. The relatively short amount of time required to generate a baculovirus stock that is compatible with cGMP use, in comparison to a cell line, is particularly advantageous for the rapid rollout of updated vaccines targeting current circulating variants.

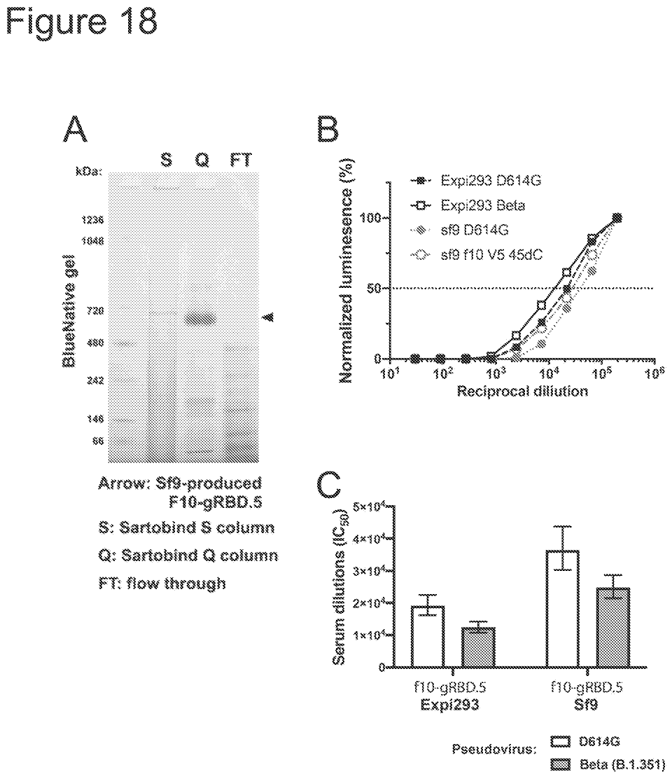

F10-gRBD can be efficiently expressed and purified from a baculovirus/Sf9-cell expression system. F10-gRBD.1 and F10gRBD.5 versions (see Table 3) were efficiently expressed in the baculovirus/Sf9 system. The potential for baculovirus/Sf9-expressed F10-gRBD.5 to be purified without relying on a sequence tag also was assessed. A two-step column purification was performed, first with a Sartobind S column to remove cellular and baculoviral fragments, and second with a Sartobind Q anion exchange column. This approach for tag-free purification efficiently isolated the F10-gRBD.5 multimer from Sf9-produced material (FIG. 18A). 85% purity without detectable loss of material was achieved before polishing with size-exclusion chromatography (SEC).

The immunogenicity of F10-gRBD.5 produced in the baculovirus/Sf9 system was compared with the immunogenicity of F10-gRBD.5 produced in Expi293 cells. F10-gRBD.5 was more immunogenic, eliciting more potent neutralizing antibody titers, when produced in Sf9 cells than when produced in Expi293 cells (FIG. 18B-C). Without the intention of being limited by any particular theory, it is conceivable that the glycan structures created by the insect Sf9 cells enhance immunogenicity. Thus, the baculovirus/Sf9 system, or insect cells in general, were found to be an optimal production platform for F10-gRBD.5.

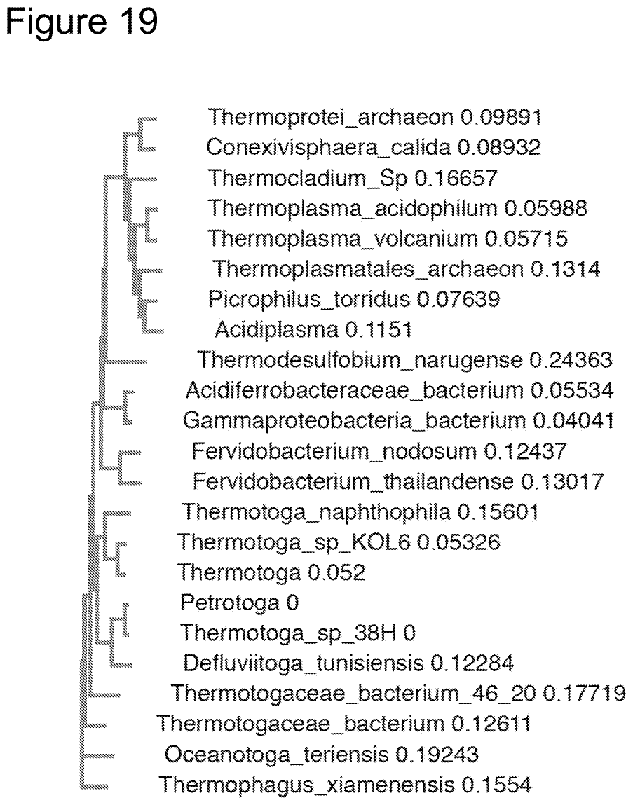

Based on the success of Acidiferrobacteraceae bacterium (Ap) half-ferritin F10 as a self-assembling multimer vaccine antigen scaffold, related protein sequences were identified. These sequences define a class of scaffolds similar and comparably advantageous to Acidiferrobacteraceae bacterium F10. Moreover, divergent half-ferritin scaffolds are particularly useful for boosting immune responses elicited first by an antigen presented on a different half-ferritin scaffold, as such a prime-boost strategy would focus the immune response away from the scaffold, i.e., by selectively boosting antibodies against the antigen. Half-ferritins (F10s) from thermophilic archaea or bacteria were of particular interest. Scaffolds based on the following thermophilic archaeal or bacterial sequences were identified, and define a class of thermophile F10 proteins. The phylogenetic relationships of these thermophile F10 proteins is shown in FIG. 19. Their phylogenetic relationships provide guidance for selecting thermophile F10 proteins with maximally divergent sequences for a prime-boost regimen designed to focus the immune response away from the scaffold and onto the antigen, selecting thermophile F10 proteins with maximally similar properties, or understanding the sequence plasticity of the thermophile F10 proteins. As with the F10 of SEQ ID NO:10, the natural thermophile F10 sequence can be modified, e.g., by replacing a cystine with another amino acid (e.g., alanine or serine). Scaffolds may be derived from any of the following F10 proteins from thermophilic archaea or bacteria:

Thermoplasma acidophilum F10 (SEQ ID NO: 174):
MPRYEVSEDLSERIKDLSRARQSLIEEIEAMMFYDERADATKDADLKHI
MEHNRDDEKEHAVLLLEWIRRHDPALDRELHEILYSEKPIKELGD
Picrophilus torridus F10 (SEQ ID NO: 178):
MPMYESGEDLSGKIRDLSRARQSLIEEMQAIMFYDERADVTKDPELKAV
IEHNRDDEKEHFSLLLEYLRRNDPQLDRELKEILFSNKPLKELGD
Thermoplasma volcanium F10 (SEQ ID NO: 175):
MPRYESGEDLSERIKDLSRARQSLIEEIEAMMFYDERADATKDEDLKYI
MEHNRDDEKEHAALLLEWIRRHDPAMDKELHEILFSNKKMKELGD
Acidiplasma F10 (SEQ ID NO: 180):
MPVYESEGSLDERTKDLSRARQSLIEEMQAIMFYDERAYATKDKNLRDV
IEHNRDDEKEHFSLLLEYLRRNDPQLDRELREILFSNKELKDLGD
Thermotogaceae bacterium F10 (SEQ ID NO: 200):
MSNYHEPFEQLSEKARDISRALNSLKEEIEAVDWYNQRVDATEDAELKS
VMAHNRDEEIEHACMTLEWLRRNMDGWDDELKTYLFTKAPITEVEEAGE
GSDNGGLNIGKMK
Thermotogaceae bacterium 46 20 F10 (SEQ ID
NO: 194):
MSAYHEPVEELSAKARDITRVLNSLKEEIEAVDWYNQRAEAASDAEAKA
IIEHNRDEEIEHAVMLLEWLRRNMDGWDEEMRTYLFTESPITEMEQSED
SNGSSKKTSGDLNIRGLRE
Thermodesulfobium narugense F10 (SEQ ID NO: 206):
MAGNMYEDPKAIGEKAMDLHRAISSLMEELEAIDYYNQRVMATTDPELK
KILIHNRDEEKEHAAMLIEYLRRVDPKFEHELKDYLFTTKDFGDMG
Fervidobacterium nodosum F10 (SEQ ID NO: 204):
MSYHEPYEELQDLDRDFSRLIRSLIEELEAIDWYNQRMSVSKDPEVKAV
VKHNRDEEMEHAAMVLEVLRRRVPELDKALRTYLFTDVPITEVEEKATE
GDTSSNNNSELIRP
Fervidobacterium thailandense F10 (SEQ ID
NO: 205):
MAYHEPYELLGDDARDLSRLLRSLIEELEAIDWYNQRMSVSKDPDVKAV
VKHNRDEEMEHAAMVLEIIRRRVPEFDKALRTYLFTEGPITEIEAASQE
GPNDDGNQLLRP
Thermotoga F10 (SEQ ID NO: 192):
MADQYHEPVSELTGKDRDFVRALNSLKEEIEAVAWYHQRVVTTKDETVR
KILEHNRDEEMEHAAMLLEWLRRNMPGWDEALRTYLFTDKPITEIEEET
SGGSENTGGDLGIRKL
Thermotoga sp KOL6 F10 (SEQ ID NO: 191):
MADQYHEPVSELSNQDRDFVRALNSLKEEIEAIAWYHQRVAATKDETVK
KILEHNRNEEMEHAAMLLEWLRRNMSGWDEALRTYLFTDKPITEIEEEE
SSGGSENSRGDLGIRKL
Thermotoga naphthophila F10 (SEQ ID NO: 190):
MAEQYHEPVDELTSKDRDFTRALVSLKEEIEAIMWYQQRASATKDQAIR
EVLEHNRDEEMEHAAMLLEWLRRNMPGWDKALRTYLFTSEPLTQIEEEA
MGGEESSSGGDLGLRKIKRG
Thermotoga sp F10 (SEQ ID NO: 185):
MQDYHEPYEELSDKDRSYVYALNSLKEEIEAIDWYNQRAAVSKDPTIKE
IMEHNRDEEIEHAVMLIEWLRRNMNGWDEELRTYLFTEKPLLEVEEEAV
EGESKVESSSNKKGDLGLRGLK
Oceanotoga teriensis F10 (SEQ ID NO: 193):
MGDYHESYDALDQRTRDLTRALNSLKEEIEAVDWYNQRVALAENEELKS
IMAHNRDEEIEHAVMTLEWLRRNMDGWDEEMKTYLFKEGNITDLEEEIE
KSEDSKDESLGIKDMNK
Defluviitoga tunisiensis F10 (SEQ ID NO: 186):
MQDYHQPYEELSQQDRSYVYALNSLKEEIEAIDWYNQRAAVSKDKTIKE
IMEHNRDEEIEHAVMIIEWLRRNMAGWDEQLRKYLFTQASLIEVEEASS
EDNESSTGDLGLRKLTDK
Gammaproteobacteria bacterium F10 (SEQ ID
NO: 220):
MSNEGYHEPISELSDETRDMHRAIVSLMEELEAVDWYNQRVDACRDEEL
KAILAHNRDEEKEHAAMVLEWIRRKDPAFDGELKDYLFTEKPIAHE
Thermophagus xiamenensis F10 (SEQ ID NO: 198):
MSNYHEPAEELSQEARNFSRALNSLKEEIEAVDWYHQRVDLTEDESLRK
IMAHNRDEEIEHACMTIEWLRRNMPGWDEELRNYLFTEGDITELEEGEN
NSTDSSAHSLGIGKIKK
Thermoplasmatales archaeon F10 (SEQ ID NO: 177):
MPRFEVSENLSKRMNDLSRARQSLIEEMEAIMFYDERADATENEDLRNV
IVHNRDDEKEHFSLLLEFLRRNDPELDRELKEILFSKKKLEELGD
Thermocladium Sp. F10 (SEQ ID NO: 172):
MPRYEELKDIDKHVVDLSRARQSLIEELEAIMFYDERISATSDESLREV
LKHNRDDEKEHASLLIEWLRRNDPEFDKELREKLFTKKPLSELGD
Thermoprotei archaeon F10 (SEQ ID NO: 169):
MNGSASVEDLNRARQSLIEELQAIMWYDARAKEVEDGELRGVIAHNRDD
EKEHATLLLEWIRRHDPAMDRELREILFSGKPLSGMGD
Conexivisphaera calida F10 (SEQ ID NO: 170):
MDESVEDLNRARQSLIEELQAMMWYDQRIKETEDEELRSVLAHNRDDEK
EHASLILEWIRRHDRAMDRELREILFSAKKLSEMGD.

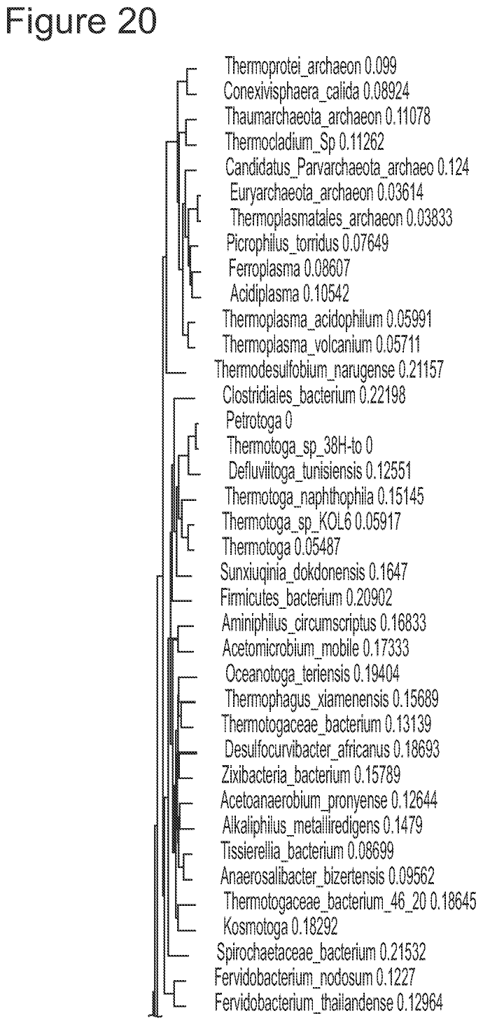

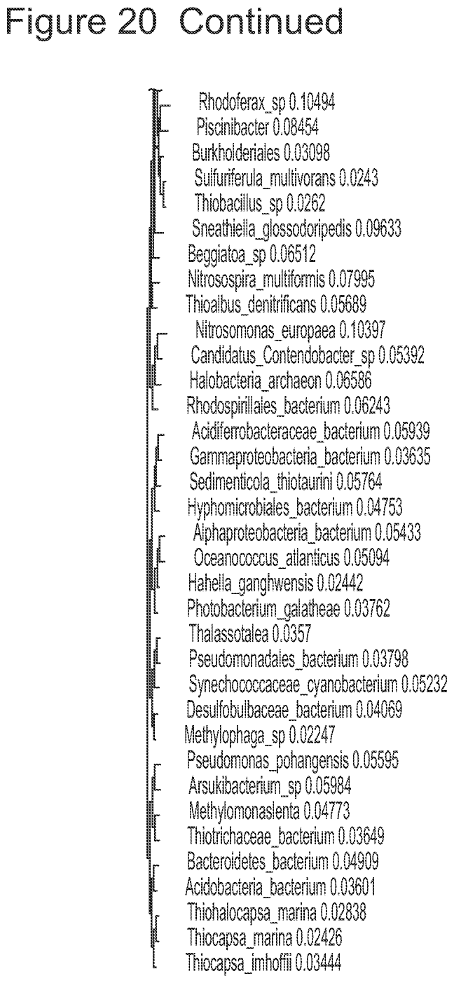

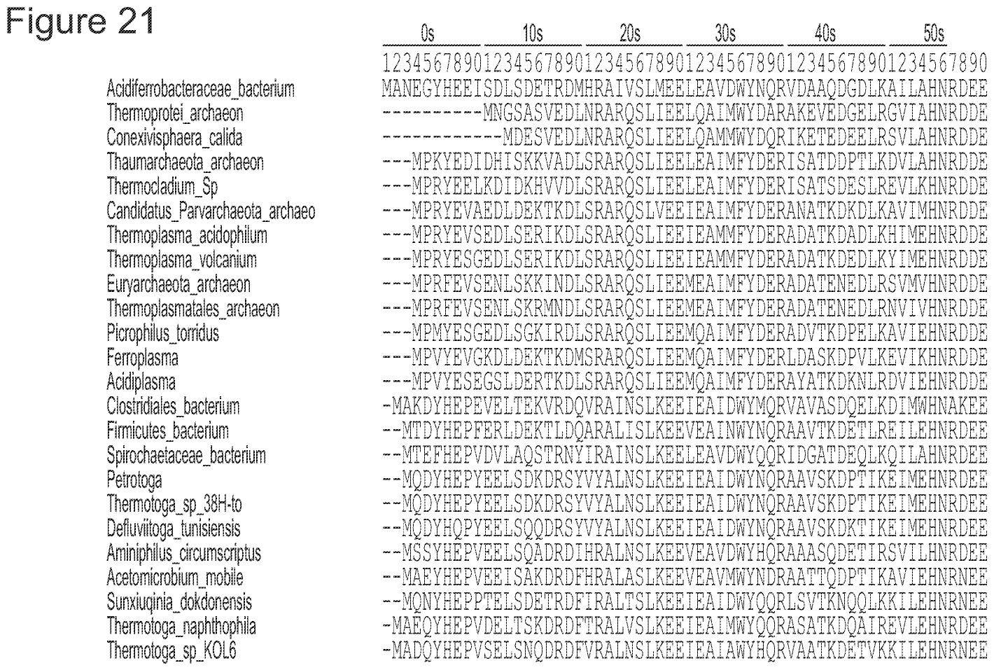

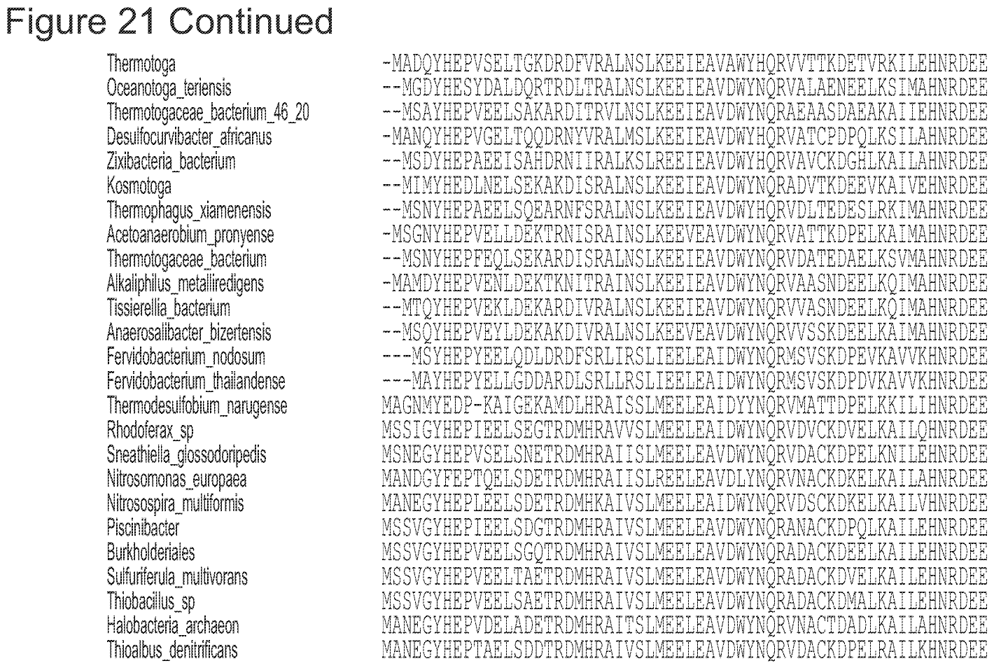

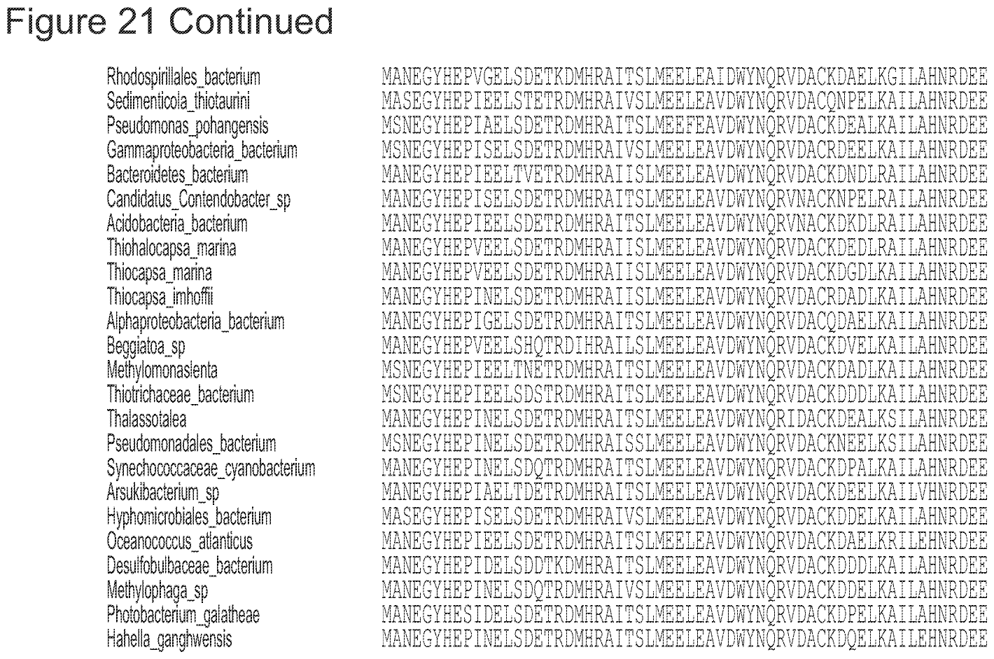

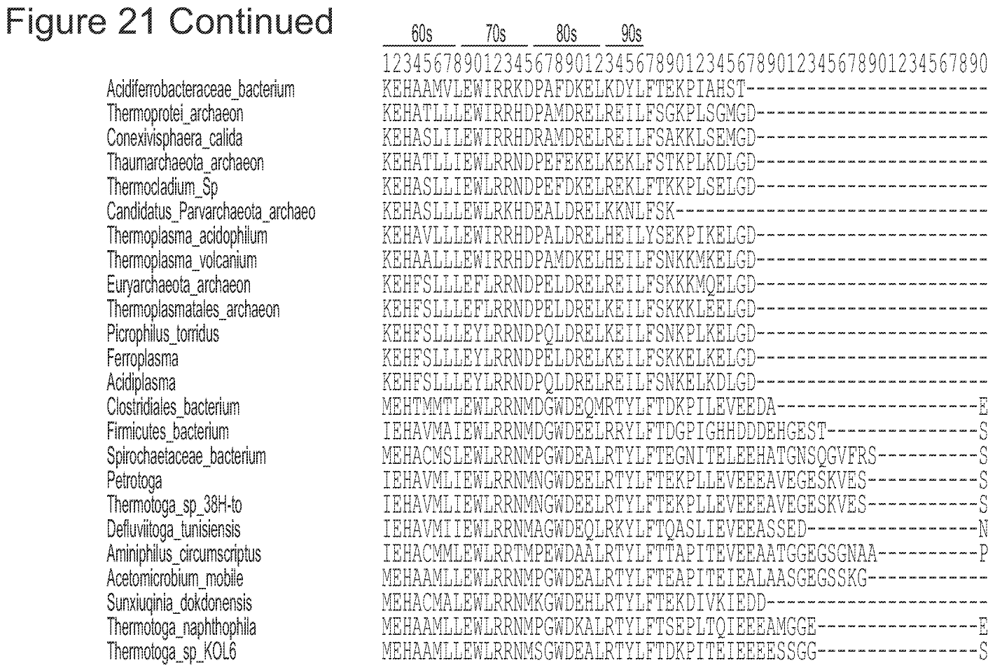

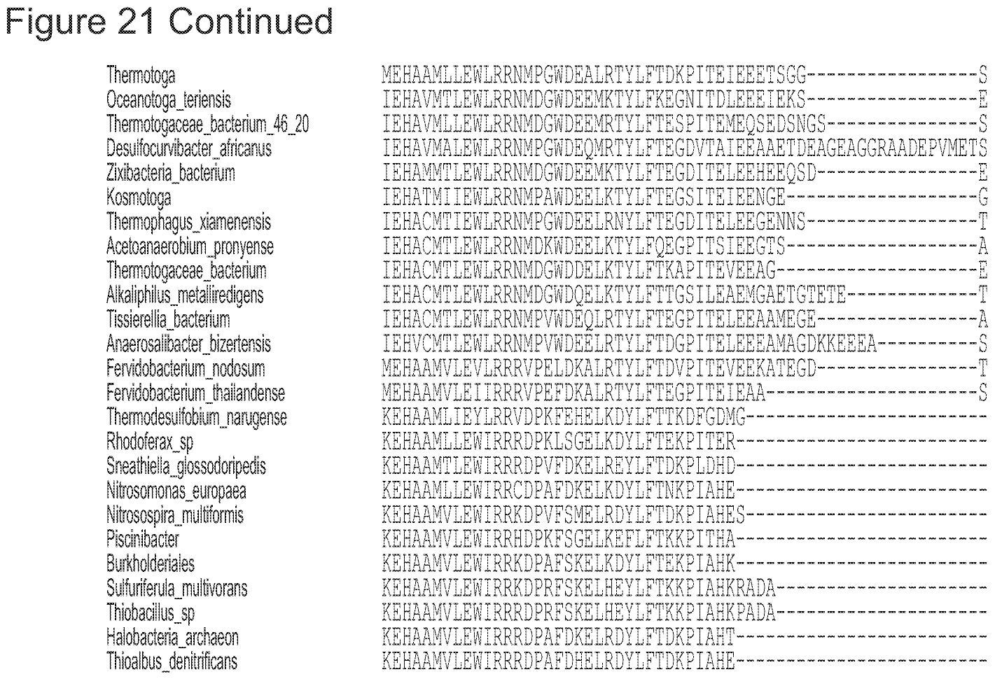

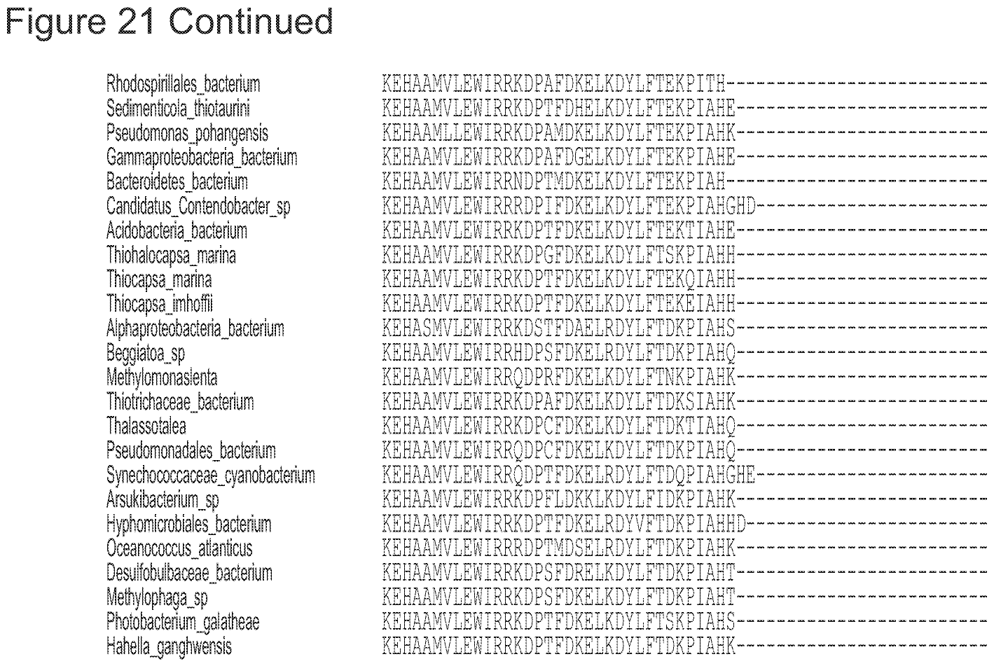

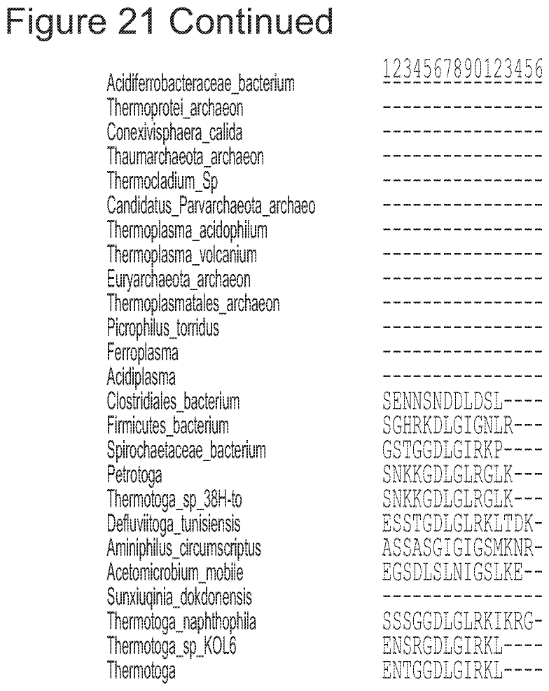

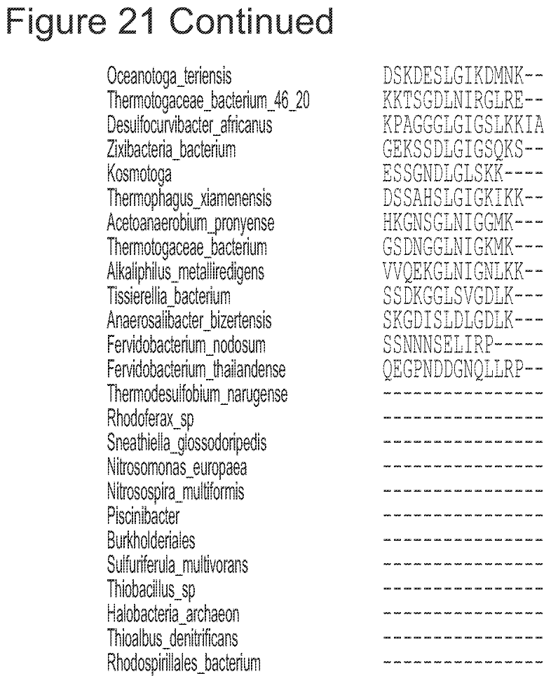

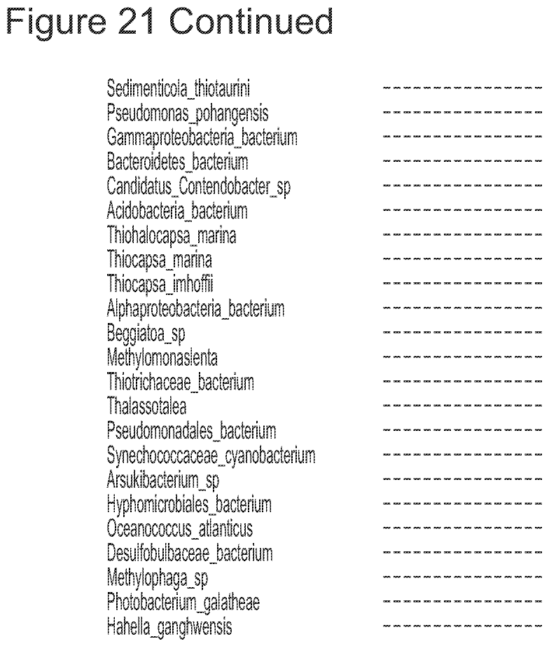

Useful F10 proteins are not limited to thermophiles. Scaffolds based on the following archaeal or bacterial sequences were identified, and define a broader class of F10 proteins than that limited to thermophile F10 proteins. The phylogenetic relationships of various F10 protein sequences, including the thermophile F10 protein sequences, is shown in FIG. 20. These phylogenetic relationships provide guidance for selecting F10 proteins with maximally divergent sequences for a prime-boost regimen designed to focus the immune response away from the scaffold and onto the antigen, selecting F10 proteins with maximally similar properties, and understanding the sequence plasticity of the F10 proteins. A multiple sequence alignment for the prokaryotic F10 proteins in SEQ ID NOs:169-240 is presented in FIG. 21. This multiple sequence alignment provides guidance for understanding the sequence plasticity of F10 proteins and/or identifying similar or divergent F10 sequences. As with the F10 of SEQ ID NO:10, the natural F10 sequence can be modified, e.g., by replacing a cystine with another amino acid (e.g., alanine or serine). Likewise, the N-terminal methionine can be deleted or replaced, e.g., when adding an N-terminal signal sequence for secretion into the endoplasmic reticulum (ER) of a eukaryotic cell. F10 scaffolds can be derived from the following prokaryotic F10 proteins:

Nitrosomonas europaea F10 (SEQ ID NO: 209):
MANDGYFEPTQELSDETRDMHRAIISLREELEAVDLYNQRVNACKDKEL
KAILAHNRDEEKEHAAMLLEWIRRCDPAFDKELKDYLFTNKPIAHE
Thiocapsa marina F10 (SEQ ID NO: 225):
MANEGYHEPVEELSDETRDMHRAIISLMEELEAVDWYNQRVDACKDGDL
KAILAHNRDEEKEHAAMVLEWIRRKDPTFDKELKDYLFTEKQIAHH
Thiohalocapsa marina F10 (SEQ ID NO: 224):
MANEGYHEPVEELSDETRDMHRAIISLMEELEAVDWYNQRVDACKDEDL
RAILAHNRDEEKEHAAMVLEWIRRKDPGFDKELKDYLFTSKPIAHH
Methylophaga sp. F10 (SEQ ID NO: 238):
MANEGYHEPINELSDQTRDMHRAIVSLMEELEAVDWYNQRVDACKDDEL
KAILAHNRDEEKEHAAMVLEWIRRKDPSFDKELKDYLFTDKPIAHT
Photobacterium galatheae F10 (SEQ ID NO: 239):
MANEGYHESIDELSDETRDMHRAITSLMEELEAVDWYNQRVDACKDPEL
KAILAHNRDEEKEHAAMVLEWIRRKDPTFDKELKDYLFTSKPIAHS
Thiocapsa imhoffii F10 (SEQ ID NO: 226):
MANEGYHEPINELSDETRDMHRAIISLMEELEAVDWYNQRVDACRDADL
KAILAHNRDEEKEHAAMVLEWIRRKDPTFDKELKDYLFTEKEIAHH
Rhodospirillales bacterium F10 (SEQ ID NO: 217):
MANEGYHEPVGELSDETKDMHRAITSLMEELEAIDWYNQRVDACKDAEL
KGILAHNRDEEKEHAAMVLEWIRRKDPAFDKELKDYLFTEKPITH
Desulfobulbaceae bacterium F10 (SEQ ID NO: 237):
MANEGYHEPIDELSDDTKDMHRAITSLMEELEAVDWYNQRVDACKDDDL
KAILAHNRDEEKEHAAMVLEWIRRKDPSFDRELKDYLFTDKPIAHT
Hahella ganghwensis F10 (SEQ ID NO: 240):
MANEGYHEPINELSDETRDMHRAITSLMEELEAVDWYNQRVDACKDQEL
KAILEHNRDEEKEHAAMVLEWIRRKDPTFDKELKDYLFTDKPIAHK
Hyphomicrobiales bacterium F10 (SEQ ID NO: 235):
MASEGYHEPISELSDETRDMHRAIVSLMEELEAVDWYNQRVDACKDDEL
KAILAHNRDEEKEHAAMVLEWIRRKDPTFDKELRDYVFTDKPIAHHD
Halobacteria archaeon F10 (SEQ ID NO: 215):
MANEGYHEPVDELADETRDMHRAITSLMEELEAVDWYNQRVNACTDADL
KAILAHNRDEEKEHAAMVLEWIRRRDPAFDKELRDYLFTDKPIAHT
Candidatus Contendobacter sp. F10 (SEQ ID
NO: 222):
MANEGYHEPISELSDETRDMHRAITSLMEELEAVDWYNQRVNACKNPEL
RAILAHNRDEEKEHAAMVLEWIRRRDPIFDKELKDYLFTEKPIAHGHD
Alphaproteobacteria bacterium F10 (SEQ ID
NO: 227):
MANEGYHEPIGELSDETRDMHRAITSLMEELEAVDWYNQRVDACQ
DAELKAILAHNRDEEKEHASMVLEWIRRKDSTFDAELRDYLFTDKPIAH
S
Sedimenticola thiotaurini F10 (SEQ ID NO: 218):
MASEGYHEPIEELSTETRDMHRAIVSLMEELEAVDWYNQRVDACQNPEL
KAILAHNRDEEKEHAAMVLEWIRRKDPTFDHELKDYLFTEKPIAHE
Methylomonaslenta F10 (SEQ ID NO: 229):
MSNEGYHEPIEELTNETRDMHRAITSLMEELEAVDWYNQRVDACKDADL
KAILAHNRDEEKEHAAMVLEWIRRQDPRFDKELKDYLFTNKPIAHK
Pseudomonadales bacterium F10 (SEQ ID NO: 232):
MSNEGYHEPINELSDETRDMHRAISSLMEELEAVDWYNQRVDACKNEEL
KSILAHNRDEEKEHAAMVLEWIRRQDPCFDKELKDYLFTDKPIAHQ
Pseudomonas pohangensis F10 (SEQ ID NO: 219):
MSNEGYHEPIAELSDETRDMHRAITSLMEEFEAVDWYNQRVDACKDEAL
KAILAHNRDEEKEHAAMLLEWIRRKDPAMDKELKDYLFTEKPIAHK
Synechococcaceae cyanobacterium F10 (SEQ ID
NO: 233):
MANEGYHEPINELSDQTRDMHRAITSLMEELEAVDWYNQRVDACKDPAL
KAILAHNRDEEKEHAAMVLEWIRRQDPTFDKELRDYLFTDQPIAHGHE
Thalassotalea F10 (SEQ ID NO: 231):
MANEGYHEPINELSDETRDMHRAITSLMEELEAVDWYNQRIDACKDEAL
KSILAHNRDEEKEHAAMVLEWIRRKDPCFDKELKDYLFTDKTIAHQ
Acidobacteria bacterium F10 (SEQ ID NO: 223):
MANEGYHEPIEELSDETRDMHRAITSLMEELEAVDWYNQRVNACKDKDL
RAILAHNRDEEKEHAAMVLEWIRRKDPTFDKELKDYLFTEKTIAHE
Thioalbus denitrificans F10 (SEQ ID NO: 216):
MANEGYHEPTAELSDDTRDMHRAIVSLMEELEAVDWYNQRVDACKDPEL
RAILKHNRDEEKEHAAMVLEWIRRRDP AFDHELRDYLFTDKPIAHE
Nitrosospira multiformis F10 (SEQ ID NO: 210):
MANEGYHEPLEELSDETRDMHKAIVSLMEELEAIDWYNQRVDSCKDKEL
KAILVHNRDEEKEHAAMVLEWIRRKDPVFSMELRDYLFTDKPIAHES
Beggiatoa sp. F10 (SEQ ID NO: 228):
MANEGYHEPVEELSHQTRDIHRAILSLMEELEAVDWYNQRVDACKDVEL
KAILAHNRDEEKEHAAMVLEWIRRHDPSFDKELRDYLFTDKPIAHQ
Thiotrichaceae bacterium F10 (SEQ ID NO: 230):
MSNEGYHEPIEELSDSTRDMHRAITSLMEELEAVDWYNQRVDACKDDDL
KAILAHNRDEEKEHAAMVLEWIRRKDPAFDKELKDYLFTDKSIAHK
Arsukibacterium sp. F10 (SEQ ID NO: 234):
MANEGYHEPIAELTDETRDMHRAITSLMEELEAVDWYNQRVDACKDEEL
KAILVHNRDEEKEHAAMVLEWIRRKDPFLDKKLKDYLFIDKPIAHK
Acetomicrobium mobile F10 (SEQ ID NO: 188):
MAEYHEPVEEISAKDRDFHRALASLKEEVEAVMWYNDRAATTQDPTIKA
VIEHNRNEEMEHAAMLLEWLRRNMPGWDEALRTYLFTEAPITEIEALAA
SGEGSSKGEGSDLSLNIGSLKE
Tissierellia bacterium F10 (SEQ ID NO: 202):
MTQYHEPVEKLDEKARDIVRALNSLKEEIEAVDWYNQRVVASNDEELKQ
IMAHNRDEEIEHACMTLEWLRRNMPVWDEQLRTYLFTEGPITELEEAAM
EGEASSDKGGLSVGDLK
Anaerosalibacter bizertensis F10 (SEQ ID NO: 203):
MSQYHEPVEYLDEKAKDIVRALNSLKEEVEAVDWYNQRVVSSKDEELKA
IMAHNRDEEIEHVCMTLEWLRRNMPVWDEELRTYLFTDGPITELEEEAM
AGDKKEEEASSKGDISLDLGDLK
Firmicutes bacterium F10 (SEQ ID NO: 182):
MTDYHEPFERLDEKTLDQARALISLKEEVEAINWYNQRAAVTKDETLRE
ILEHNRDEEIEHAVMAIEWLRRNMDGWDEELRRYLFTDGPIGHHDDDEH
GESTSSGHRKDLGIGNLR
Aminiphilus circumscriptus F10 (SEQ ID NO: 187):
MSSYHEPVEELSQADRDIHRALNSLKEEVEAVDWYHQRAAASQDETIRS
VILHNRDEEIEHACMMLEWLRRTMPEWDAALRTYLFTTAPITEVEEAAT
GGEGSGNAAPASSASGIGIGSMKNR
Desulfocurvibacter africanus F10 (SEQ ID NO: 195):
MANQYHEPVGELTQQDRNYVRALMSLKEEIEAVDWYHQRVATCPDPQLK
SILAHNRDEEIEHAVMALEWLRRNMPGWDEQMRTYLFTEGDVTAIEEAA
ETDEAGEAGGRAADEPVMETSKPAGGGLGIGSLKKIA
Zixibacteria bacterium F10 (SEQ ID NO: 196):
MSDYHEPAEEISAHDRNIIRALKSLREEIEAVDWYHQRVAVCKDGHLKA
ILAHNRDEEIEHAMMTLEWLRRNMDGWDEEMKTYLFTEGDITELEEHEE
QSDEGEKSSDLGIGSQKS
Alkaliphilus metalliredigens F10 (SEQ ID NO: 201):
MAMDYHEPVENLDEKTKNITRAINSLKEEIEAVDWYNQRVAASNDEELK
QIMAHNRDEEIEHACMTLEWLRRNMDGWDQELKTYLFTTGSILEAEMGA
ETGTETETVVQEKGLNIGNLKK
Sunxiuginia dokdonensis F10 (SEQ ID NO: 189):
MQNYHEPPTELSDETRDFIRALTSLKEEIEAIDWYQQRLSVTKNQQLKK
ILEHNRNEEMEHACMALEWLRRNMKGWDEHLRTYLFTEKDIVKIEDD
Clostridiales bacterium F10 (SEQ ID NO: 181):
MAKDYHEPEVELTEKVRDQVRAINSLKEEIEAIDWYMQRVAVASDQELK
DIMWHNAKEEMEHTMMTLEWLRRNMDGWDEQMRTYLFTDKPILEVEEDA
ESENNSNDDLDSL
Spirochaetaceae bacterium F10 (SEQ ID NO:183):
MTEFHEPVDVLAQSTRNYIRAINSLKEELEAVDWYQQRIDGATDEQLKQ
ILAHNRDEEMEHACMSLEWLRRNMPGWDEALRTYLFTEGNITELEEHAT
GNSQGVFRSSGSTGGDLGIRKP
Acetoanaerobium pronyense F10 (SEQ ID NO: 199):
MSGNYHEPVELLDEKTRNISRAINSLKEEVEAVDWYNQRVATTKDPELK
AIMAHNRDEEIEHACMTLEWLRRNMDKWDEELKTYLFQEGPITSIEEGT
SAHKGNSGLNIGGMK
Kosmotoga F10 (SEQ ID NO: 197):
MIMYHEDLNELSEKAKDISRALNSLKEEIEAVDWYNQRADVTKDEEVKA
IVEHNRDEEIEHATMIIEWLRRNMPAWDEELKTYLFTEGSITEIEENGE
GESSGNDLGLSKK
Euryarchaeota archaeon F10 (SEQ ID NO: 176):
MPRFEVSENLSKKINDLSRARQSLIEEMEAIMFYDERADATENEDLRSV
MVHNRDDEKEHFSLLLEFLRRNDPELDRELREILFSKKKMQELGD
Candidatus Parvarchaeota archaeon F10 (SEQ ID
NO: 173):
MPRYEVAEDLDEKTKDLSRARQSLVEEIEAIMFYDERANATKDKDL
KAVIMHNRDDEKEHASLLLEWLRKHDEALDRELKKNLFSK
Ferroplasma F10 (SEQ ID NO: 179):
MPVYEVGKDLDEKTKDMSRARQSLIEEMQAIMFYDERLDASKDPVLKEV
IKHNRDDEKEHFSLLLEYLRRNDPELDRELKEILFSKKELKELGD
Thaumarchaeota archaeon F10 (SEQ ID NO: 171):
MPKYEDIDHISKKVADLSRARQSLIEELEAIMFYDERISATDDPTLKDV
LAHNRDDEKEHATLLIEWLRRNDPEFEKELKEKLFSTKPLKDLGD
Burkholderiales F10 (SEQ ID NO: 212):
MSSVGYHEPVEELSGQTRDMHRAIVSLMEELEAVDWYNQRADACKDEEL
KAILEHNRDEEKEHAAMVLEWIRRKDPAFSKELKDYLFTEKPIAHK
Sulfuriferula multivorans F10 (SEQ ID NO: 213):
MSSVGYHEPVEELTAETRDMHRAIVSLMEELEAVDWYNQRADACKDVEL
KAILEHNRDEEKEHAAMVLEWIRRKDPRFSKELHEYLFTKKPIAHKRAD
A
Piscinibacter F10 (SEQ ID NO: 211):
MSSVGYHEPIEELSDGTRDMHRAIVSLMEELEAVDWYNQRANACKDPQL
KAILEHNRDEEKEHAAMVLEWIRRHDPKFSGELKEFLFTKKPITHA
Oceanococcus atlanticus F10 (SEQ ID NO: 236):
MANEGYHEPIEELSDETRDMHRAITSLMEELEAVDWYNQRVDACKDAEL
KRILEHNRDEEKEHAAMVLEWIRRRDPTMDSELRDYLFTDKPIAHK
Thiobacillus sp. F10 (SEQ ID NO: 214):
MSSVGYHEPVEELSAETRDMHRAIVSLMEELEAVDWYNQRADACKDMAL
KAILEHNRDEEKEHAAMVLEWIRRRDPRFSKELHEYLFTKKPIAHKPAD
A
Rhodoferax sp. F10 (SEQ ID NO: 207):
MSSIGYHEPIEELSEGTRDMHRAVVSLMEELEAIDWYNQRVDVCKDVEL
KAILQHNRDEEKEHAAMLLEWIRRRDPKLSGELKDYLFTEKPITER
Bacteroidetes bacterium F10 (SEQ ID NO: 221):
MANEGYHEPIEELTVETRDMHRAIISLMEELEAVDWYNQRVDACKDNDL
RAILAHNRDEEKEHAAMVLEWIRRNDPTMDKELKDYLFTEKPIAH
Sneathiella glossodoripedis F10 (SEQ ID NO: 208):
MSNEGYHEPVSELSNETRDMHRAIISLMEELEAVDWYNQRVDACKDPEL
KNILEHNRDEEKEHAAMTLEWIRRRDPVFDKELREYLFTDKPLDHD.

The invention thus has been disclosed broadly and illustrated in reference to representative embodiments described above. It is understood that various modifications can be made to the present invention without departing from the spirit and scope thereof.

It is further noted that all publications, sequence accession numbers, patents and patent applications cited herein are hereby expressly incorporated by reference in their entirety and for all purposes as if each is individually so denoted. Definitions that are contained in text incorporated by reference are excluded to the extent that they contradict definitions in this disclosure.

What is claimed is:

1. An engineered antigen or multimer thereof, comprising an altered receptor-binding domain (RBD) sequence of SARS-CoV-2 spike (S) protein that has modifications relative to the wildtype RBD sequence, wherein the modifications comprise mutations at the inter-subunit interfaces of the RBD that result in (a) formation of at least two engineered N-linked glycosylation sites, (b) formation of at least one engineered N-linked glycosylation site and substitution of at least one additional hydrophobic residue at the inter-subunit interface, or (c) formation of at least one engineered N-linked glycosylation site that is formed from two substitutions.

2. The antigen or multimer of claim 1, wherein the wildtype RBD sequence comprises residues N331-P527 (SEQ ID NO:2) or a substantially identical or conservatively modified variant thereof, wherein mutations that result in the formation of an N-linked engineered glycosylation site comprise V362(S/T), L517N/H519(S/T), A520N/P521X/A522(S/T), A372T, A372S, Y396T, D428N, R357N/S359T, R357N/S359S, S371N/S373T, S371N/S373S, S383N/P384V, S383N/P384A, S383N/P384I, S383N/P384L, S383N/P384M, S383N/P384W, K386N/N388T, K386N/N388S, and G413N, and wherein the amino acid numbering is based on SARS-CoV-2 S protein sequence of Access No. YP_009724390.1 (SEQ ID NO:1), and X is any amino acid except for P.

3. The antigen or multimer of claim 2, wherein substitution of at least one additional hydrophobic residue comprises substitution of residue V362, V367, A372, L390, L455, L517, L518, A520, P521, or A522 with a charged amino acid residue.

4. The antigen or multimer of claim 2, wherein the mutations comprise (a) any two of A372(T/S) and L517N/H519(T/S), (b) L517N/H519(T/S) and D428N, (c) any three of A372(T/S), Y396T, D428N, and L517N/H519(T/S), (d) any two of A372(T/S), Y396T, D428N, and L517N/H519(T/S), plus substitution of L518; (e) any two of A372(T/S), Y396T, and D428N, plus substitution of L517; (f) L517N/H519(T/S), plus substitution of V372, (g) L517N/H519(T/S), plus substitution of L390, or (h) any two of V362(S/T), A372(S/T), D428N, L517N/H519(T/S), A520N/P521X/A522(S/T), wherein X is any amino acid except for P.

5. The antigen or multimer of claim 2, comprising substitutions L517N/H519T or L517N/H519S in the wildtype RBD sequence (SEQ ID NO:2).

6. The antigen or multimer of claim 5, further comprising one or more substitutions selected from the group consisting of D428N, A372(T/S), Y396T, V372(D/E), L390(D/E), L455A, and L518(D/E/G/S).

7. The antigen or multimer of any one of claims 1-6, further comprising two or more substitutions selected from the group consisting of V362(S/T), D428N, L518(D/E/G/S).

8. The antigen or multimer of claim 2, comprising the amino sequence shown in any one of SEQ ID NOs:3, 162-168 and 241-246, or a substantially identical or conservatively modified variant thereof.

9. The antigen or multimer of any one of claims 1-8, which does not comprise a full-length SARS-CoV-2 spike (S) protein.

10. A fusion protein, comprising the antigen of any one of claims 1-9 and at least part of a heterologous protein.

11. The fusion protein of claim 10, comprising a transmembrane region or a glycosylphosphatidylinositol (GPI) anchor signal sequence.

12. The fusion protein of claim 11, wherein the heterologous protein is a self-assembling multimer scaffold protein.

13. A fusion protein comprising an antigen and a scaffold protein, wherein the scaffold protein is at least 50% (e.g., at least 60%, at least 65%, at least 70%, at least 75%, at least 80%, at least 85%, at least 90%, at least 95%, at least 96%, at least 97%, or at least 98%) identical to amino acids 2-96 of Acidiferrobacteraceae bacterium (Ap) half-ferritin (SEQ ID NO: 10).

14. The fusion protein of claim 13, wherein the C-terminus of the scaffold protein is fused (a) to the N-terminus of the antigen directly, (b) to the N-terminus of the antigen through a polypeptide linker, or (c) to the antigen via an isopeptide bond.

15. The fusion protein of any one of claims 1-14, comprising the sequence shown in SEQ ID NO:10, or a substantially identical or conservatively modified variant thereof.

16. A fusion protein comprising an antigen and a scaffold protein, wherein the scaffold protein is at least 50% (e.g., at least 60%, at least 65%, at least 70%, at least 75%, at least 80%, at least 85%, at least 90%, at least 95%, at least 96%, at least 97%, or at least 98%) identical to the F10 protein sequence shown in any one of SEQ ID NOs:169-240.

17. The fusion protein of any one of claims 13-16, comprising the sequence shown in any one of SEQ ID NOs:169-240, or a substantially identical or conservatively modified variant thereof.

18. A fusion protein comprising an antigen and a scaffold protein, wherein (a) the scaffold protein is a self-assembling homo-multimer comprising 10-59 subunits; and (b) the C-terminus of the scaffold protein is fused (i) to the N-terminus of the antigen directly, or (ii) to the N-terminus of the antigen through a polypeptide linker.

19. A fusion protein comprising an antigen and a scaffold protein, wherein (a) the scaffold protein is a self-assembling homo-multimer comprising 13-59 subunits; and (b) the C-terminus of the scaffold protein is fused (i) to the N-terminus of the antigen directly, (ii) to the N-terminus of the antigen through a polypeptide linker, or (iii) to the antigen via an isopeptide bond; and wherein self-assembly of the scaffold protein is not dependent upon cysteine coordination of a metal ion or binding to nucleic acid.

20. The fusion protein of any one of claims 13-19, wherein the antigen comprises an altered receptor-binding domain (RBD) sequence of SARS-CoV-2 spike (S) protein that has modifications relative to the wildtype RBD sequence, wherein the modifications comprise mutations at the inter-subunit interfaces of the RBD that result in (a) formation of at least two engineered N-linked glycosylation sites or (b) formation of at least one engineered N-linked glycosylation site and substitution of at least one additional hydrophobic residue at the inter-subunit interface.

21. The fusion protein of any one of claims 10-20, comprising an N-terminal signal sequence for secretion into the endoplasmic reticulum (ER) of a eukaryotic cell.

22. The fusion protein of any one of claims 12-21, wherein the scaffold protein is not a heat-shock protein.

23. The fusion protein of any one of claims 18-22, wherein the scaffold protein is a self-assembling homo-multimer comprising 24-48 subunits.

24. The fusion protein of any one of claims 12-23, wherein the scaffold protein is a substantially identical or conservatively modified variant of a protein from a prokaryote.

25. The fusion protein of any one of claims 12-24, wherein the scaffold protein is a substantially identical or conservatively modified variant of a protein from a thermophile or hyperthermophile.

26. The fusion protein of any one of claims 12-25, wherein the scaffold protein is an imidazoleglycerol-phosphate dehydratase (HisB) protein or a substantially identical or conservatively modified variant thereof.

27. The fusion protein of any one of claims 10-26, wherein the scaffold protein comprises at least one N-linked glycan.

28. The fusion protein of claim 27, comprising at least one N-linked glycan (a) in the region corresponding to positions 1-59 of SEQ ID NO:34 or (b) at the position corresponding to 12 of SEQ ID NO:34.

29. The fusion protein of any one of claims 18-28, wherein the scaffold protein is an ATP-dependent Clp protease proteolytic subunit (ClpP) protein, a catalytically-inactive ClpP protein, or a substantially identical or conservatively modified variant thereof.

30. The fusion protein of claim 29, comprising a valine at the position corresponding to A140 of SEQ ID NO:97.

31. The fusion protein of any one of claims 13-30, wherein the scaffold protein comprises the sequence shown in any one of SEQ ID NO:4-10 and 34-154, or a substantially identical or conservatively modified variant thereof.

32. The fusion protein of any one of claims 10-12, comprising the sequence shown in any one of SEQ ID NOs:11-22, or a substantially identical or conservatively modified variant thereof.

33. A vaccine composition comprising two or more distinct versions of the fusion protein of any one of claims 10-32.

34. A polynucleotide that encodes the antigen of any one of claims 1-9 or the fusion protein of any one of claims 10-32.

35. The polynucleotide of claim 34, wherein said polynucleotide is a ribonucleic acid (RNA).

36. A SARS-CoV-2 vaccine composition, comprising the antigen of any one of claims 1-9, the fusion protein of any one of claims 10-32, or the polynucleotide of any one of claims 34-35.

37. The SARS-CoV-2 vaccine composition of claim 35, comprising two or more distinct versions of the antigen of any one of claims 1-9, two or more distinct versions of the fusion protein of any one of claims 10-32, or two or more distinct versions of the polynucleotide of any one of claims 34-35.

38. A pharmaceutical composition, comprising the vaccine composition of claim 33 or 37, and a pharmaceutically acceptable carrier.
